(19) United States
(12) Patent Application Publication
(10) Pub. No.: US 2024/0391895 A1
(43) Pub. Date: Nov. 28, 2024

(54) PYRIDAZINE-CONTAINING COMPOUND AND MEDICINAL USE THEREOF

(71) Applicant: Tuojie Biotech (Shanghai) Co., Ltd., Shanghai (CN)

(72) Inventors: Jian Yu, Shanghai (CN); Xiaming Pang, Shanghai (CN); Yunfei Li, Shanghai (CN); Fang Zhang, Shanghai (CN)

(21) Appl. No.: 18/568,114

(22) PCT Filed: Dec. 24, 2021
(86) PCT No.: PCT/CN2021/141211

§ 371 Date: Dec. 7, 2023

(30) Foreign Application Priority Data
Dec. 25, 2020 (CN) 202011562172.X
Jan. 22, 2021 (CN) 202110090687.2
Feb. 8, 2021 (CN) 202110172665.0
May 28, 2021 (CN) 202110592769.7
Jul. 13, 2021 (CN) 202110791592.3

(51) Int. Cl. C07D 401/12; A61K 31/501; A61K 31/502; A61K 31/5025; C07D 237/20; C07D 401/14; C07D 471/04; C07D 491/048; C07D 491/052

(52) U.S. Cl. CPC C07D 401/12; A61K 31/501; A61K 31/502; A61K 31/5025; C07D 237/20; C07D 401/14; C07D 471/04; C07D 491/048; C07D 491/052

(57) ABSTRACT

A pyridazine-containing compound and a medicinal use thereof. Specifically, a compound represented by formula I or a medicinal salt thereof. The compound or the medicinal salt thereof has an NLRP3 inflammasome inhibiting activity, and can be used for treating or preventing NLRP3-related diseases.

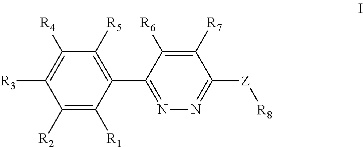

### TECHNICAL FIELD

The present disclosure relates to the field of pharmaceutics and particularly to pyridazine-containing compounds and use thereof in pharmaceutics.

### BACKGROUND

The NOD-like receptor protein 3 (NLRP3) is a protein-coding gene: the protein belongs to the family of nucleotide-binding and oligomerization domain-like receptors (NLRs) and is also known as “pyrin domain-containing protein 3” (Inoue et al., Immunology, 2013, 139, 11-18). The gene encodes a protein containing a pyridine domain, a nucleotide-binding site domain (NBD) and a leucine-rich repeat (LRR) motif. In response to sterile inflammatory danger signals, NLRP3 interacts with an adaptor protein, apoptosis-associated speck-like protein (ASC) and zymogen-1 to form the NLRP3 inflammasome. NLRP3 inflammasome activation then leads to the release of the inflammatory cytokines IL-1b and IL-18, and when dysregulated, can cause many diseases.

Studies have shown that NLRP3 inflammasome activation is associated with a variety of diseases, including: inflammasome-related diseases, immune diseases, inflammatory diseases, autoimmune diseases and autoinflammatory diseases. Therefore, there is a need to provide new NLRP3 inflammasome pathway inhibitors to provide new options for treating the disease described above.

### SUMMARY

In a first aspect, the present disclosure provides a compound of formula I or a pharmaceutically acceptable

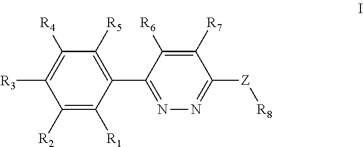

wherein R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN; R₂, R₃, R₄, R₅, R₆ and R₇:
(a) R₂ and R₃, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or R₆ and R₇, together with the atoms to which they are attached, form a 5-6 membered cyclic hydrocarbon, a 5-6 membered heterocyclic ring, phenyl or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(b) R₃ and R₄, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(c) R₄ and R₅, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or, (d) R₆ and R₇, together with the atoms to which they are attached, form a 5-6 membered cyclic hydrocarbon, a 5-6 membered heterocyclic ring, phenyl or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O) NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens;
when R₆ and R₇, together with the atoms to which they are attached, form an unsubstituted 5-6 membered cyclic hydrocarbon, when R₁ is methyl or methoxy, and when Z is —NH—,
R₈ is not aryl.

In some embodiments, R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN;
R₂, R₃, R₄, R₅, R₆ and R₇:
(a) R₂ and R₃, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(b) R₃ and R₄, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(c) R₄ and R₅, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or, (d) R₆ and R₇, together with the atoms to which they are attached, form a 5-6 membered cyclic hydrocarbon, a 5-6 membered heterocyclic ring, phenyl or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O) NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens;
when R₆ and R₇, together with the atoms to which they are attached, form an unsubstituted 5-6 membered cyclic hydrocarbon, when R₁ is methyl or methoxy, and when Z is —NH—, R₈ is not aryl.

In some embodiments, R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN;
R₂, R₃, R₄, R₅, R₆ and R₇:
(a) R₂ and R₃, together with the atoms to which they are attached, form a 5-6 membered cyclic hydrocarbon, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(b) R₃ and R₄, together with the atoms to which they are attached, form a 5-6 membered cyclic hydrocarbon or phenyl that is optionally substituted with one or more substituents selected from the group consisting of: deuterium, halogen, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(c) R₄ and R₅, together with the atoms to which they are attached, form a 5-6 membered cyclic hydrocarbon or phenyl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or, (d) R₆ and R₇, together with the atoms to which they are attached, form a 5-6 membered cyclic hydrocarbon, a 5-6 membered heterocyclic ring or phenyl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R_{8b}, —S(═O) NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens;
when R₆ and R₇, together with the atoms to which they are attached, form an unsubstituted 5-6 membered cyclic hydrocarbon, when R₁ is methyl or methoxy, and when Z is —NH—, R₈ is not aryl.

In some embodiments, R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(CO)NH—C₁₋₆ alkyl, and the substituents are selected from the group consisting of: deuterium, halogen, —OH, —NH₂ and —CN;
R₂, R₃, R₄, R₅, R₆ and R₇:
(a) R₂ and R₃, together with the atoms to which they are attached, form a 5-6 membered cyclic hydrocarbon, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(b) R₃ and R₄, together with the atoms to which they are attached, form a 5-6 membered cyclic hydrocarbon or phenyl that is optionally substituted with one or more substituents selected from the group consisting of: deuterium, halogen, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(c) R₄ and R₅, together with the atoms to which they are attached, form a 5-6 membered cyclic hydrocarbon that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or, (d) R₆ and R₇, together with the atoms to which they are attached, form a 5-6 membered cyclic hydrocarbon, a 5-6 membered heterocyclic ring or phenyl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O) NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens; when R₆ and R₇, together with the atoms to which they are attached, form an unsubstituted 5-6 membered cyclic hydrocarbon, when R₁ is methyl or methoxy, and when Z is —NH—, R₈ is not aryl.

In some embodiments, R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN;
R₂, R₃, R₄, R₅, R₆ and R₇:
(a) R₂ and R₃, together with the atoms to which they are attached, form a 5-6 membered cyclic hydrocarbon, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN; (b) R₃ and R₄, together with the atoms to which they are attached, form a 5-6 membered cyclic hydrocarbon that is optionally substituted with one or more substituents selected from the group consisting of: deuterium, halogen, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
(c) R₄ and R₅, together with the atoms to which they are attached, form a 5-6 membered cyclic hydrocarbon that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
or, (d) R₆ and R₇, together with the atoms to which they are attached, form a 5-6 membered cyclic hydrocarbon or a heterocyclic ring that is substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₄ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O) NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens; when R₆ and R₇, together with the atoms to which they are attached, form an unsubstituted 5-6 membered cyclic hydrocarbon, when R₁ is methyl or methoxy, and when Z is —NH—, R₈ is not aryl.

In some embodiments, R₁ is selected from the group consisting of halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN.

In some embodiments, R₁ is selected from the group consisting of —OH, —NH₂, and the following groups that are optionally substituted with one or more substituents: C₁₋₆ alkyl, —O—C₁₋₆ alkyl, and —NHC(═O)—C₁₋₆ alkyl, and the substituents are selected from the group consisting of: deuterium, halogen, —OH, —NH₂ and —CN.

In some embodiments, R₁ is selected from the group consisting of —OH, —NH₂, and the following groups that are optionally substituted with one or more —OH: C₁₋₆ alkyl, —O—C₁₋₆ alkyl and —NHC(═O)—C₁₋₆ alkyl.

In one embodiment, R₁ is —OH.

In some embodiments, R₁ is —O—C₁₋₆ alkyl, preferably —OCH₃ or —OCH₂CH₃, and more preferably —OCH₃.

In some embodiments, R₁ is C₁₋₆ alkyl that is optionally substituted with one or more —OH; preferably, R₁ is —CH₂OH or —CH₂CH₂OH; more preferably, R₁ is —CH₂OH.

In one embodiment, R₁ is —NH₂.

In some embodiments, R₁ is —NHC(═O)—C₁₋₆ alkyl that is optionally substituted with one or more —OH; preferably, R₁ is —NH₂C(═O)CH₃.

In some embodiments, R₂ and R₃, together with the atoms to which they are attached, form a 5-6 membered cyclic hydrocarbon that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

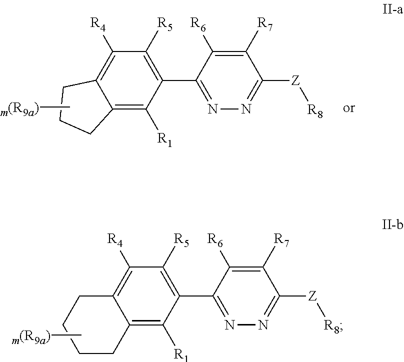

R₉ₐ, is independently selected from the group consisting of hydrogen, deuterium, halogen, —OH, —NH₂, —CN, oxo, C₁₋₆ alkyl, —O—C₁₋₆ alkyl and C₃₋₆ cycloalkyl, and the C₁₋₆ alkyl, O—C₁₋₆ alkyl and C₃₋₆ cycloalkyl are optionally further substituted with one or more deuterium atoms or halogens; m is an integer selected from 0-8.

In some embodiments, the compound of formula I is selected from the group consisting of:

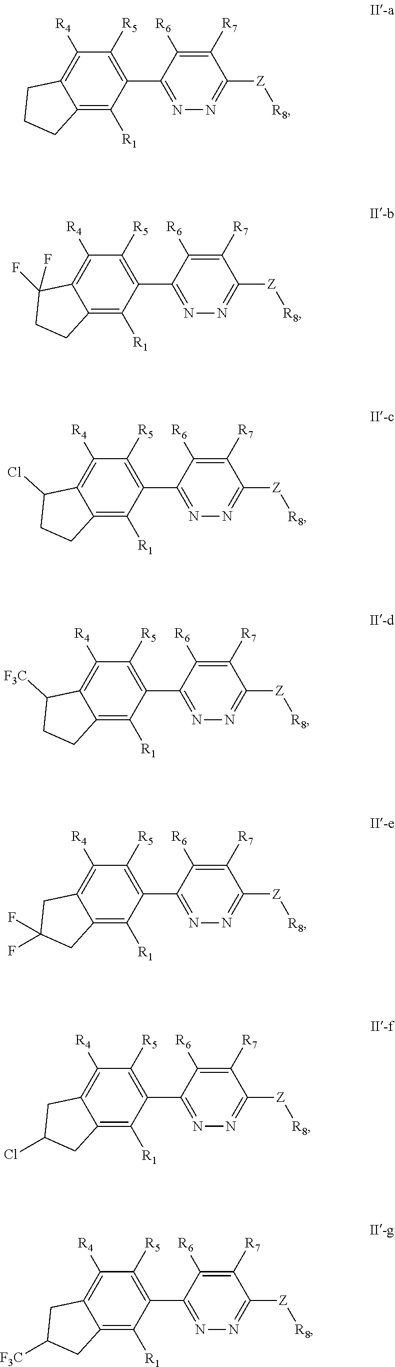

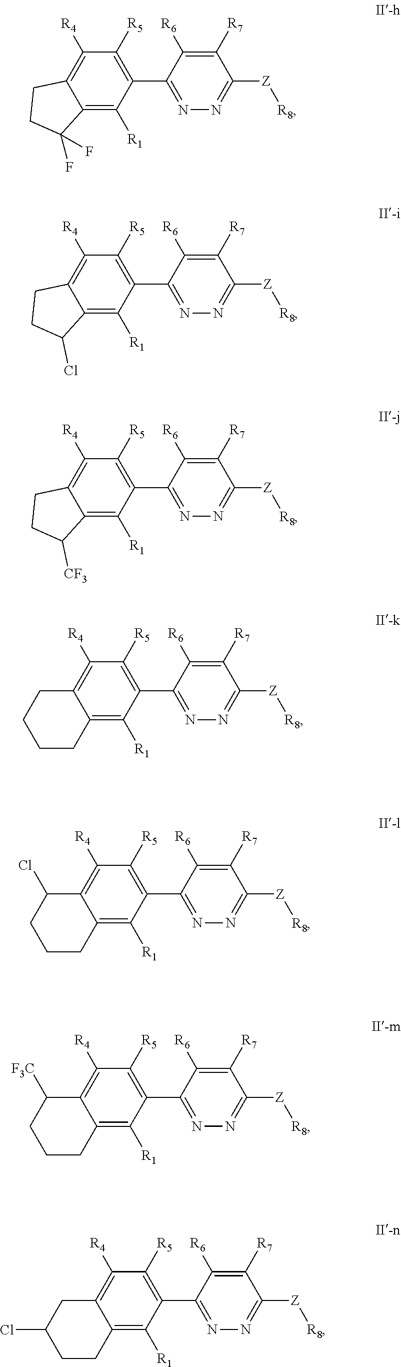

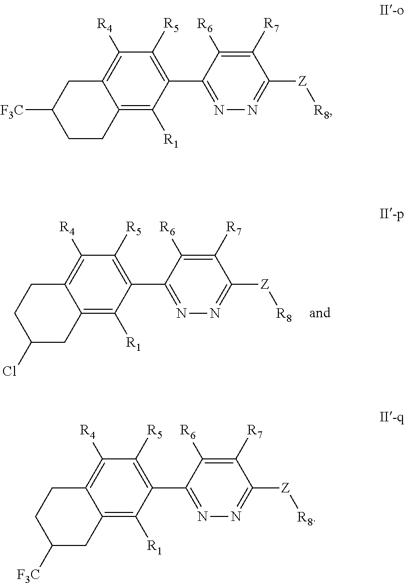

In some embodiments, the compound of formula I is selected from the group consisting of compounds of formulas II′-a, II′-b, II′-c, II′-d, II′-f, II′-g, II′-k, II′-l and II′-m, preferably compounds of formulas II′-a, II′-c, II′-d, II′-k, II′-l and II′-m, and more preferably compounds of formulas II′-a, II′-d, II′-k and II′-m, and is most preferably a compound of formula II′-a or II′-k.

In some other embodiments, R₂ and R₃, together with the atoms to which they are attached, form a 5-6 membered cyclic hydrocarbon that is optionally substituted with one or more substituents selected from the group consisting of: deuterium, halogen, —OH, —NH₂, —CN, oxo, C₁₋₄ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₆ and R₇, together with the atoms to which they are attached, form phenyl or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, R₂ and R₃, together with the atoms to which they are attached, form a 5-6 membered cyclic hydrocarbon; preferably, the 5-6 membered cyclic hydrocarbon is cyclopentyl or cyclohexyl; the cyclopentyl or cyclohexyl is optionally substituted with a substituent selected from the group consisting of hydrogen, deuterium, halogen, —OH, C₁₋₆ alkyl and C₁₋₆ haloalkyl;
R₆ and R₇, together with the atoms to which they are attached, form phenyl or 5-6 membered heteroaryl; preferably, the 5-6 membered heteroaryl is pyridine; the phenyl or 5-6 membered heteroaryl is optionally substituted with a substituent selected from the group consisting of hydrogen, deuterium, halogen, —OH, C₁₋₆ alkyl and C₁₋₆ haloalkyl;
R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN; preferably, R₄ and R₅ are independently selected from the group consisting of hydrogen, deuterium, halogen, —OH, C₁₋₆ alkyl and C₁₋₆ haloalkyl; more preferably, R₄ and R₅ are independently selected from the group consisting of hydrogen and deuterium. In some other embodiments, R₂ and R₃, together with the atoms to which they are attached, form a 5-6 membered heterocyclic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, the 5-6 membered heterocyclic ring is a 5-6 membered S-containing heterocyclic ring.

In some embodiments, the compound of formula I is selected from the group consisting of:

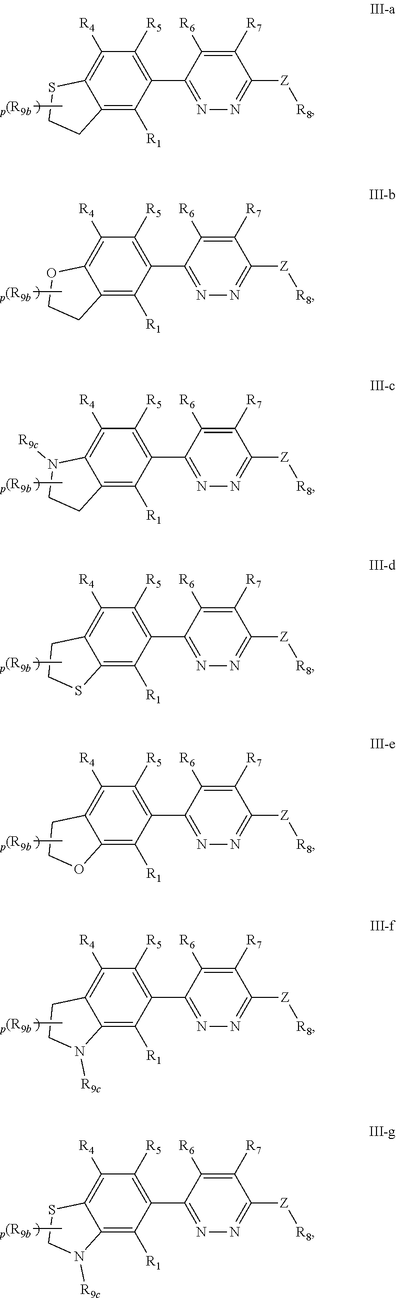

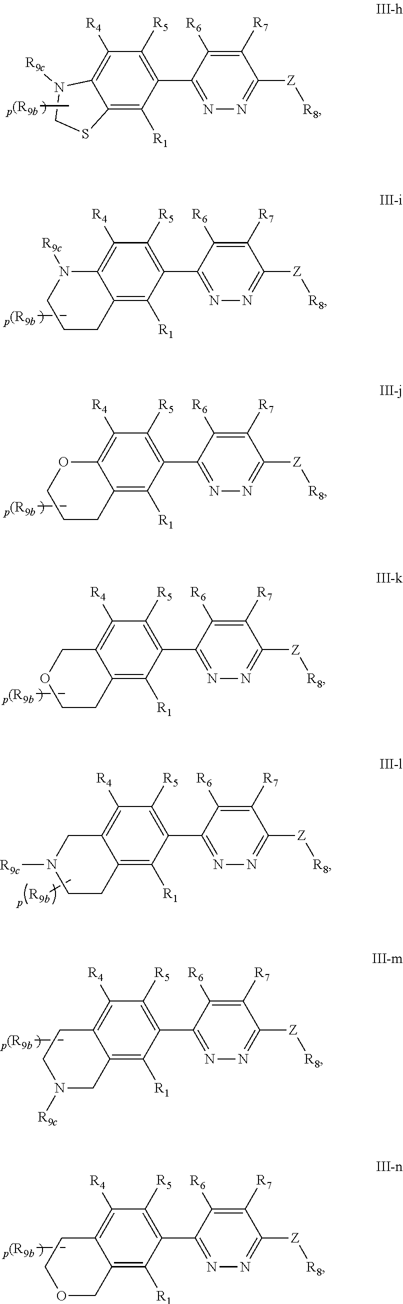

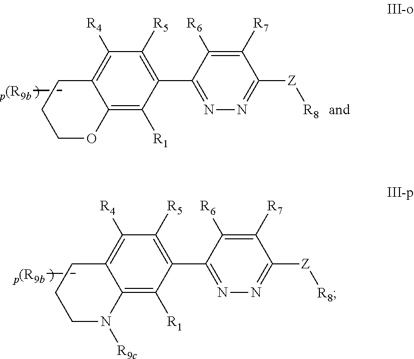

R_{9b} is selected from the group consisting of hydrogen, deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R_{9c}, is selected from the group consisting of hydrogen, deuterium, C₁₋₆ alkyl and C₃₋₆ cycloalkyl, and the C₁₋₆ alkyl and C₃₋₆ cycloalkyl are optionally further substituted with one or more deuterium atoms or halogens; p is an integer selected from 0-6.

In some embodiments, the compound of formula I is selected from the group consisting of:

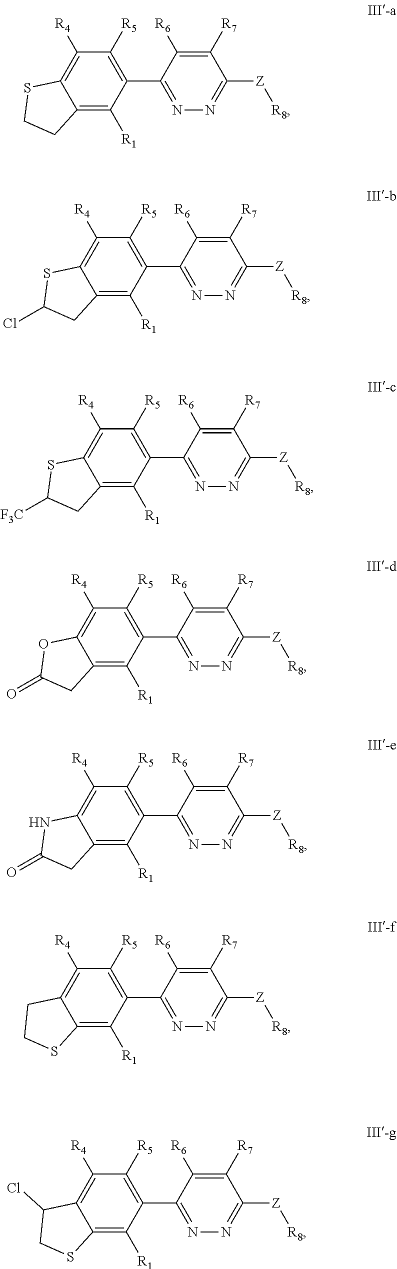

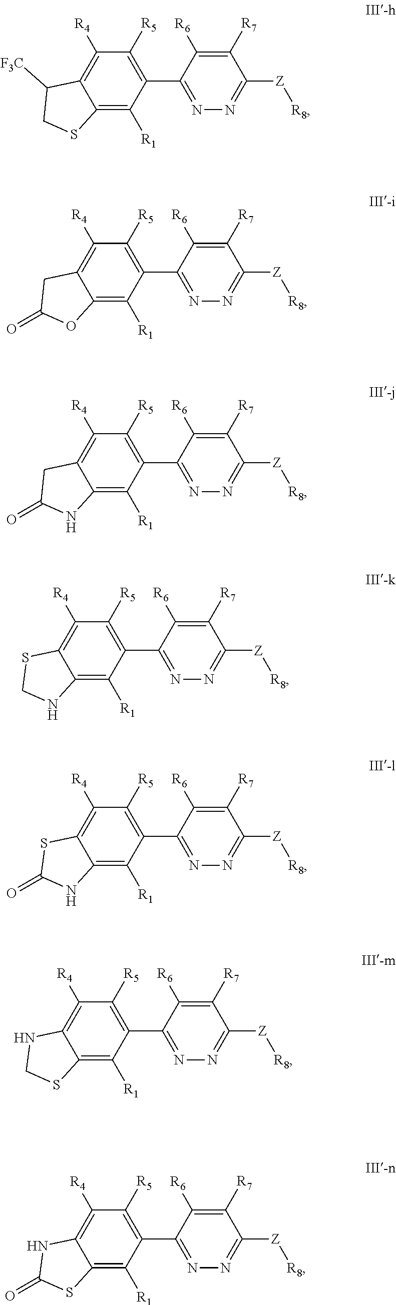

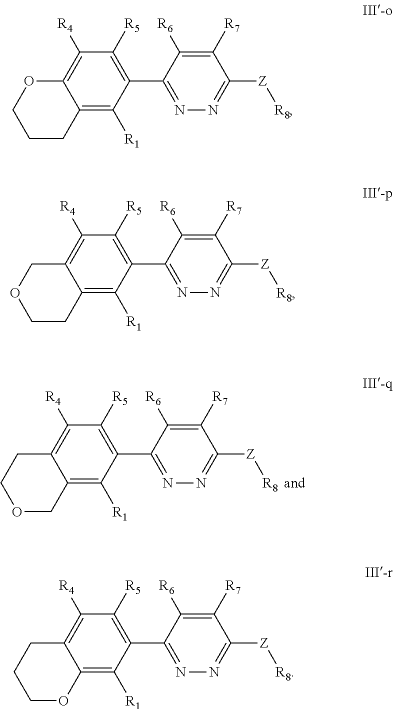

In some embodiments, the compound of formula I is selected from the group consisting of compounds of formulas III′-a, III′-b, III′-c, III′-d, III′-f, III′-g, III′-h and III′-i, preferably compounds of formulas III′-a, III′-b, III′-c, III′-f, III′-g and III′-h, and more preferably compounds of formulas III′-a, III′-c, III′-f and III′-h, and is most preferably a compound of formula III′-a or III′-f.

In some other embodiments, R₂ and R₃, together with the atoms to which they are attached, form a 5-6 membered heteroaromatic ring that is optionally substituted with one or more substituents selected from the group consisting of: deuterium, halogen, —OH, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₄, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, the 5-6 membered heteroaromatic ring is a 5-membered S-containing heteroaromatic ring.

In some embodiments, the compound of formula I is selected from the group consisting of:

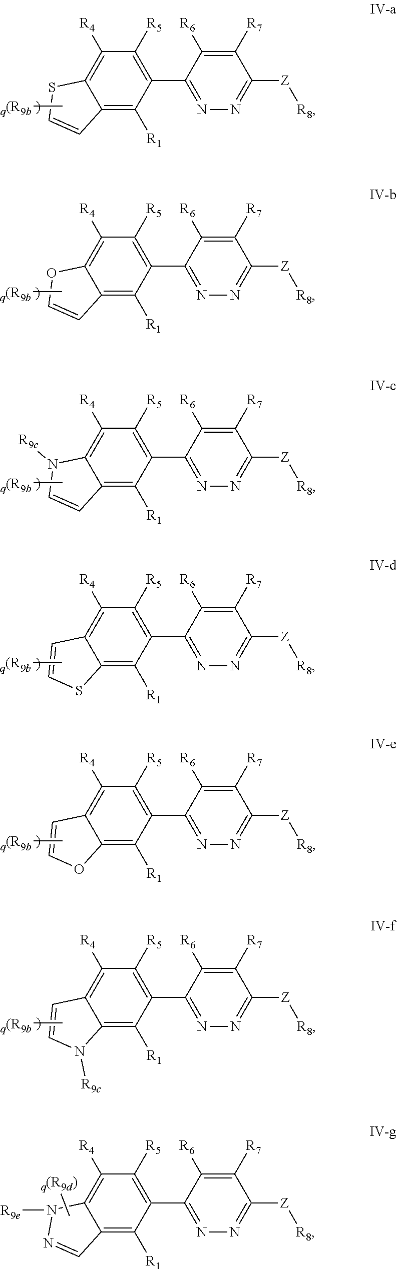

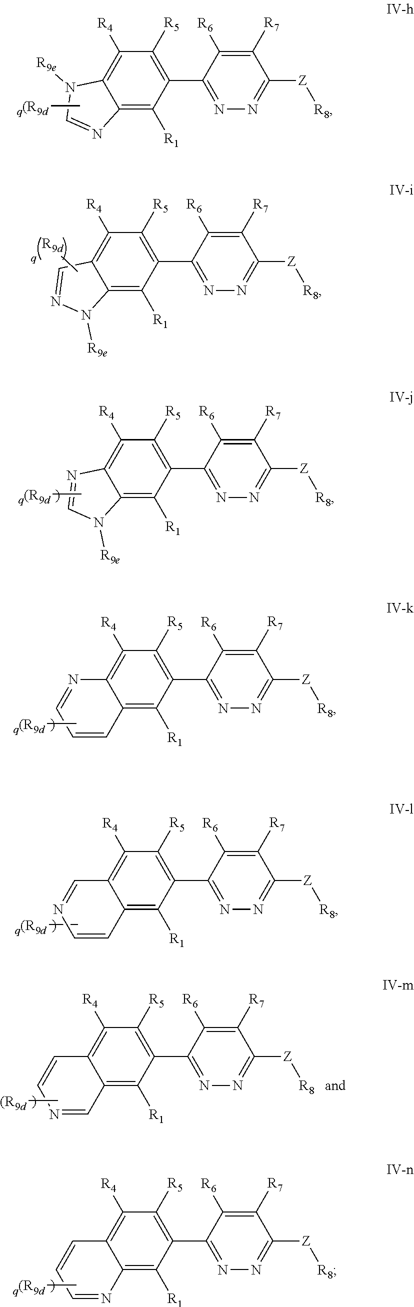

R_{9d} is selected from the group consisting of hydrogen, deuterium, halogen, —OH, —NH₂, —CN, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₉ₑ is selected from the group consisting of hydrogen, deuterium, C₁₋₆ alkyl and C₃₋₆ cycloalkyl, and the C₁₋₆ alkyl and C₃₋₆ cycloalkyl are optionally further substituted with one or more deuterium atoms or halogens; q is an integer selected from 0-3.

In some embodiments, the compound of formula I is selected from the group consisting of:

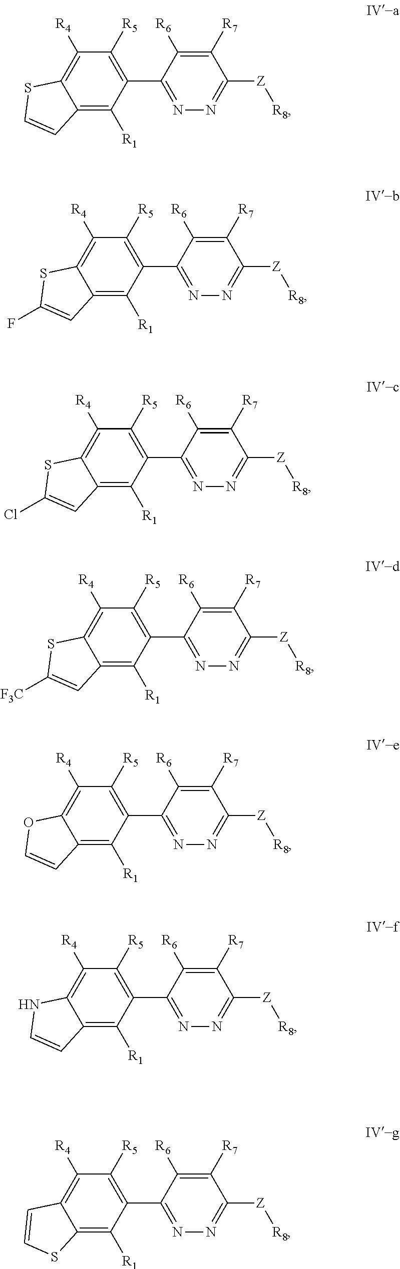

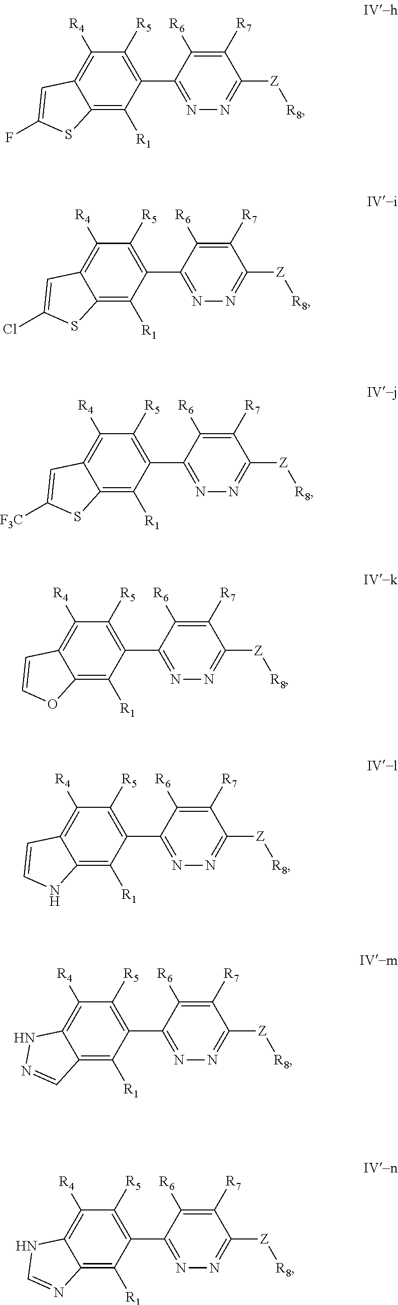

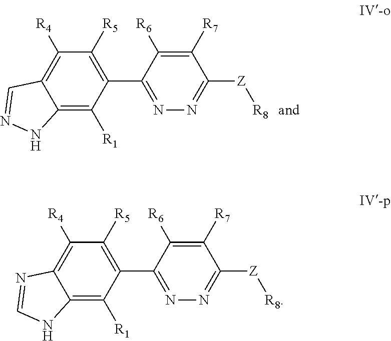

In some embodiments, the compound of formula I is selected from the group consisting of compounds of formulas IV′-a, IV′-b, IV′-c, IV′-d, IV′-e, IV′-f, IV′-g, IV′-h, IV′-i, IV′-j, IV′-k and IV′-l, preferably compounds of formulas IV′-a, IV′-b, IV′-c, IV′-d, IV′-g, IV′-h, IV′-i and IV′-j, and more preferably compounds of formulas IV′-a, IV′-d, IV′-g and IV′-j, and is most preferably a compound of formula IV′-a or IV′-g.

In some embodiments, in formula II-a, formula II-b, formulas II′-a to II′-q, formulas III-a to III-p, formulas III′-a to III′-r, formulas IV-a to IV-n and formulas IV′-a to IV′-p, R₄, R₅, R₆ and R₁ are independently selected from the group consisting of hydrogen, deuterium, halogen, and the following groups that are optionally substituted with one or more substituents: C₁₋₆ alkyl, C₃₋₆ cycloalkyl and C₃₋₆ cycloalkylmethylene, and the substituents are deuterium atoms or halogens.

In some embodiments, R₄ and R₇ are independently selected from the group consisting of hydrogen, deuterium and halogen, and R₅ and R₆ are independently selected from the group consisting of hydrogen, deuterium, halogen, and C₁₋₆ alkyl that is optionally substituted with one or more deuterium atoms or halogens.

In some embodiments, R₄, R₅ and R₇ are each hydrogen, and R₆ is methyl; or, R₄, R₆ and R₇ are each hydrogen, and R₅ is methyl.

In some other embodiments, R₃ and R₄, together with the atoms to which they are attached, form a 5-6 membered cyclic hydrocarbon that is optionally substituted with one or more substituents selected from the group consisting of: deuterium, halogen, C₁₋₆ alkyl and C₃₋₆ cycloalkyl, and the C₁₋₆ alkyl and C₃₋₆ cycloalkyl are optionally further substituted with one or more deuterium atoms or halogens;
R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, the compound of formula I is selected from the group consisting of:

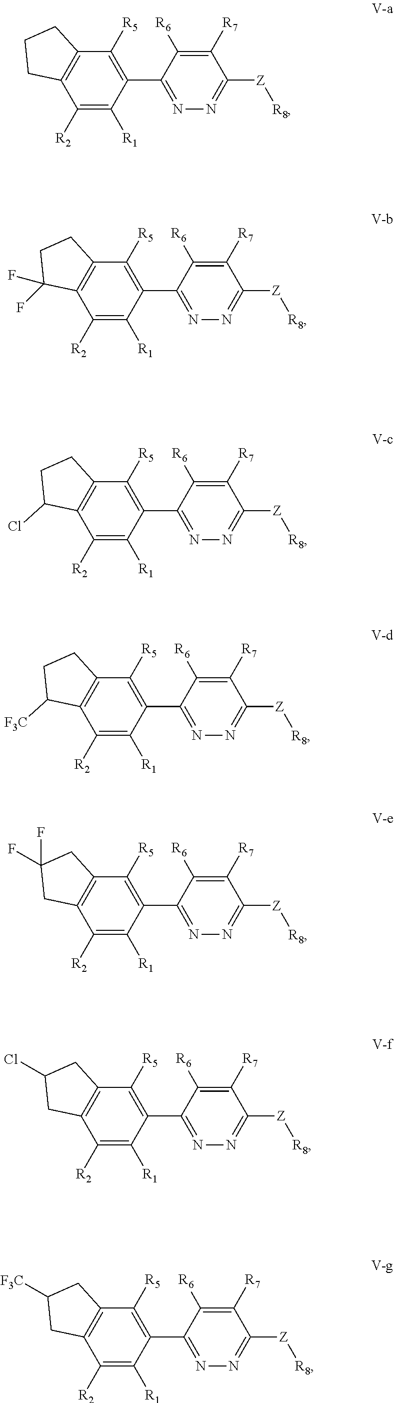

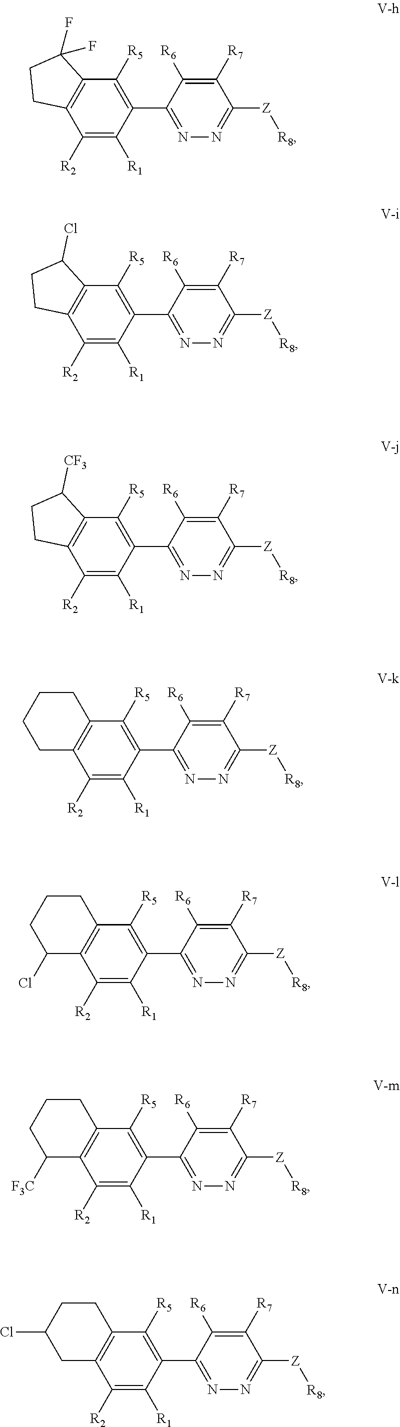

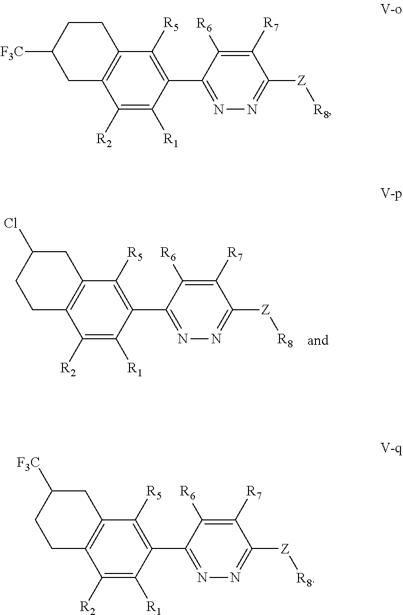

In some embodiments, the compound of formula I is selected from the group consisting of compounds of formulas V-a, V-b, V-c, V-d, V-f, V-g, V-k, V-l and V-m, preferably compounds of formulas V-a, V-c, V-d, V-k, V-l and V-m, and more preferably compounds of formulas V-a, V-d, V-k and V-m, and is most preferably a compound of formula V-a or V-k.

In some embodiments, in the compounds of formulas V-a to V-q, R₂, R₅, R₆ and R₇ are independently selected from the group consisting of hydrogen, deuterium, halogen, and the following groups that are optionally substituted with one or more substituents: C₁₋₆ alkyl, C₃₋₆ cycloalkyl and C₃₋₆ cycloalkylmethylene, and the substituents are deuterium atoms or halogens.

In some embodiments, R₂ and R₇ are independently selected from the group consisting of hydrogen, deuterium and halogen, and R₅ and R₆ are independently selected from the group consisting of hydrogen, deuterium, halogen, and C₁₋₆ alkyl that is optionally substituted with one or more deuterium atoms or halogens.

In some embodiments, R₂, R₅ and R₇ are each hydrogen, and R₆ is methyl; or, R₂, R₆ and R₇ are each hydrogen, and R₅ is methyl.

In some other embodiments, R₄ and R₅, together with the atoms to which they are attached, form a 5-6 membered cyclic hydrocarbon that is optionally substituted with one or more substituents selected from the group consisting of: deuterium, halogen, oxo, C₁₋₆ alkyl and C₃₋₆ cycloalkyl, and the C₁₋₆ alkyl and C₃₋₆ cycloalkyl are optionally further substituted with one or more deuterium atoms or halogens;
R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;

In some embodiments, the compound of formula I is selected from the group consisting

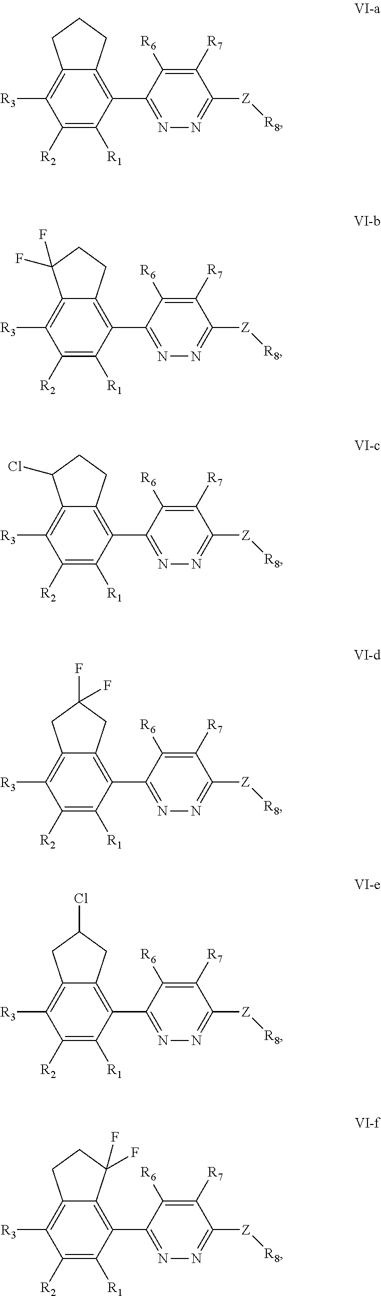

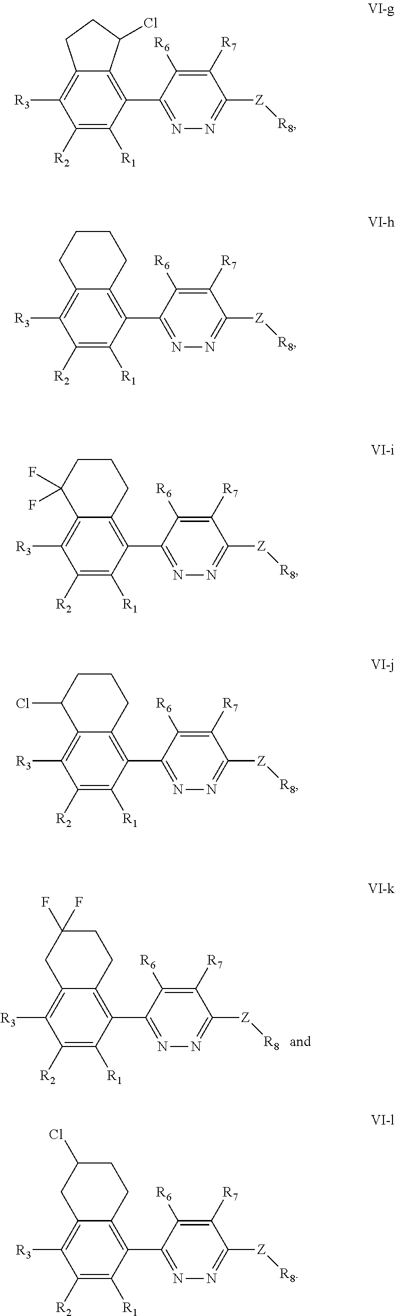

In some embodiments, the compound of formula I is selected from the group consisting of compounds of formulas VI-a, VI-b, VI-c, VI-d, VI-e, VI-h, VI-i, VI-j, VI-k and VI-l, preferably compounds of formulas VI-a, VI-b, VI-c, VI-h, VI-i and VI-j, and is most preferably a compound of formula VI-a or VI-h.

In some embodiments, in the compounds of formulas VI-a to VI-l, R₂, R₃, R₆ and R₇ are independently selected from the group consisting of hydrogen, deuterium, halogen, and the following groups that are optionally substituted with one or more substituents: C₁₋₆ alkyl, C₃₋₆ cycloalkyl and C₃₋₆ cycloalkylmethylene, and the substituents are deuterium atoms or halogens.

In some embodiments, R₂ and R₇ are independently selected from the group consisting of hydrogen, deuterium and halogen, and R₃ and R₆ are independently selected from the group consisting of hydrogen, deuterium, halogen, and C₁₋₆ alkyl that is optionally substituted with one or more deuterium atoms or halogens.

In some embodiments, R₂ and R₇ are each hydrogen, R₃ is trifluoromethyl, and R₆ is methyl; or, R₂ and R₇ are each hydrogen, R₃ is chlorine, and R₆ is methyl.

In some embodiments, R₄ and R₅, together with the atoms to which they are attached, form phenyl that is optionally substituted with one or more substituents selected from the group consisting of: deuterium, halogen, oxo, C₁₋₆ alkyl and C₃₋₆ cycloalkyl, and the C₁₋₆ alkyl and C₃₋₆ cycloalkyl are optionally further substituted with one or more deuterium atoms or halogens;
R₂, R₃, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
preferably, R₂, R₃, R₆ and R₇ are independently selected from the group consisting of hydrogen, deuterium, halogen, —OH, —NH₂, C₁₋₆ alkyl and —O—C₁₋₆ alkyl;
more preferably, R₂, R₃, R₆ and R₇ are independently selected from the group consisting of hydrogen, deuterium, halogen, —OH, —NH₂, C₁₋₆ alkyl and —O—C₁₋₆ alkyl;
most preferably, R₂, R₃, R₆ and R₇ are independently selected from the group consisting of hydrogen and deuterium.

In some embodiments, the compound of formula I is selected from the group consisting of

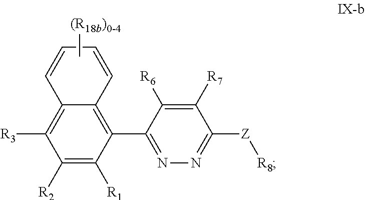

R_{18b} is independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN; preferably, R_{18b} is hydrogen.

In some embodiments, R₃ and R₄, together with the atoms to which they are attached, form phenyl that is optionally substituted with one or more substituents selected from the group consisting of: deuterium, halogen, C₁₋₆ alkyl and C₃₋₆ cycloalkyl, and the C₁₋₆ alkyl and C₃₋₆ cycloalkyl are optionally further substituted with one or more deuterium atoms or halogens;
R₂, R₅, R₆ and R₇ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
preferably, R₂, R₅, R₆ and R₇ are independently selected from the group consisting of hydrogen, deuterium, halogen, —OH, —NH₂, C₁₋₆ alkyl and —O—C₁₋₆ alkyl;
more preferably, R₂, R₅ or R₇ is independently selected from the group consisting of hydrogen, deuterium, halogen, —OH, —NH₂, C₁₋₆ alkyl and —O—C₁₋₆ alkyl; R₆ is selected from C₁₋₆ alkyl;
most preferably, R₂, R₅ and R₇ are independently selected from the group consisting of hydrogen and deuterium, and R₆ is methyl.

In some embodiments, the compound of formula I is selected from the group consisting of:

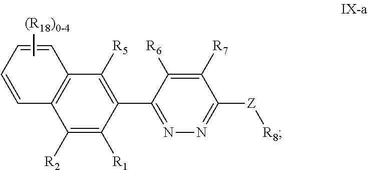

R₁₈ₐ is independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
preferably, R₁₈ₐ, is independently selected from the group consisting of hydrogen, deuterium, halogen, —OH, —NH₂ or C₁₋₆ alkyl and —O—C₁₋₆ alkyl; more preferably, R₁₈ₐ, is hydrogen.

In some other embodiments, R₆ and R₇, together with the atoms to which they are attached, form a 5-6 membered cyclic hydrocarbon that is optionally substituted with one or more substituents selected from the group consisting of: deuterium, halogen, C₁₋₆ alkyl, —O—C₁₋₆ alkyl and C₃₋₆ cycloalkyl, and the C₁₋₄ alkyl, —O—C₁₋₆ alkyl and C₃₋₆ cycloalkyl are optionally further substituted with one or more deuterium atoms or halogens; R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—Cis alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
preferably, R₂, R₄ and R₅ are independently selected from the group consisting of hydrogen, deuterium, C₁₋₆ alkyl and halogen, and R₃ is selected from the group consisting of trifluoromethyl, cyclopropyl and —S-trifluoromethyl;
more preferably, R₂, R₄ and R₅ are independently selected from the group consisting of hydrogen and deuterium, and R₃ is selected from trifluoromethyl.

In some embodiments, the compound of formula I is selected from the group consisting of:

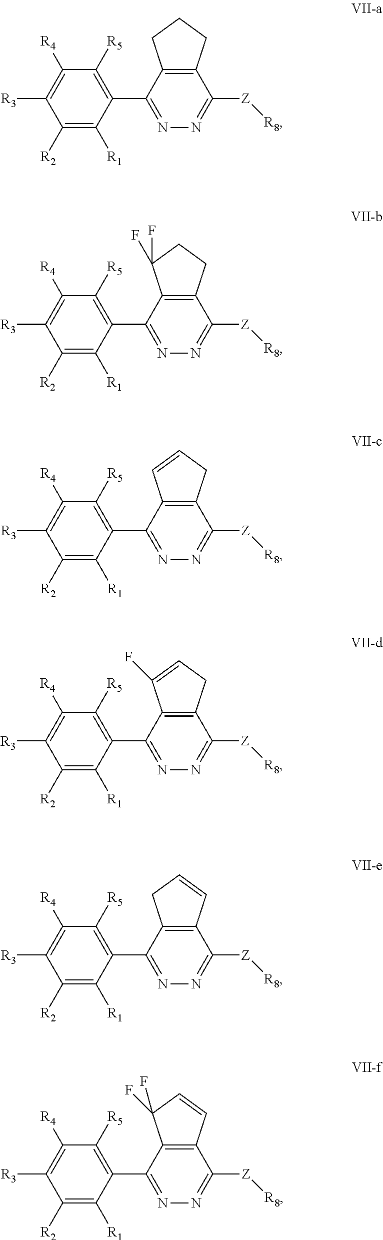

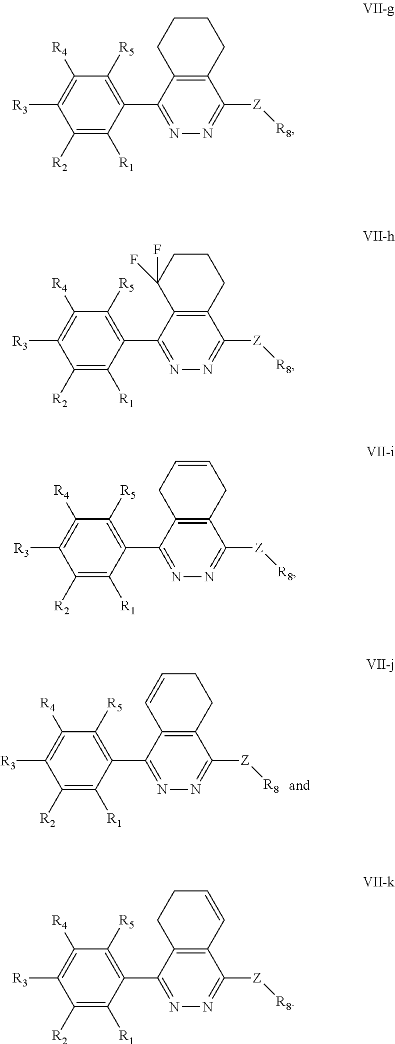

In some embodiments, the compound of formula I is selected from the group consisting of compounds of formulas VII-a, VII-b, VII-c, VII-g, VII-h and VII-i, preferably compounds of formulas VII-a, VII-b, VII-g and VII-h, and is more preferably a compound of formula VII-a or VII-g, and most preferably a compound of formula VII-g.

In some other embodiments, R₆ and R₇, together with the atoms to which they are attached, form a 5-6 membered heterocyclic ring that is optionally substituted with one or more substituents selected from the group consisting of: deuterium, halogen, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, the compound of formula I is selected from the group consisting of:

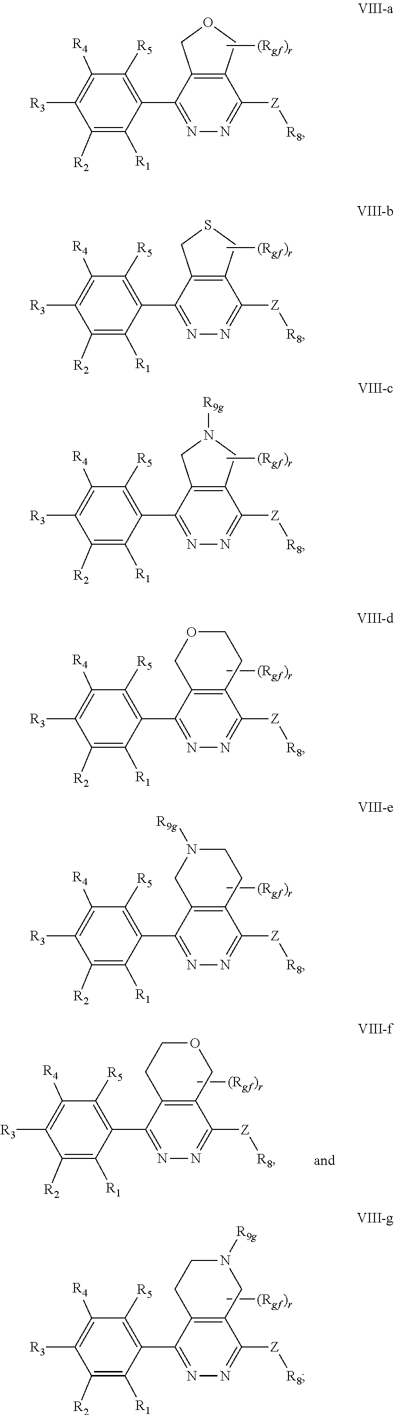

R_{9f} is independently selected from the group consisting of deuterium, halogen, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R_{9g} is selected from the group consisting of hydrogen, deuterium, C₁₋₆ alkyl and C₃₋₆ cycloalkyl, and the C₁₋₆ alkyl and C₃₋₆ cycloalkyl are optionally further substituted with one or more deuterium atoms or halogens; r is an integer selected from 0-6.

In some embodiments, the compound of formula I is selected from the group consisting of:

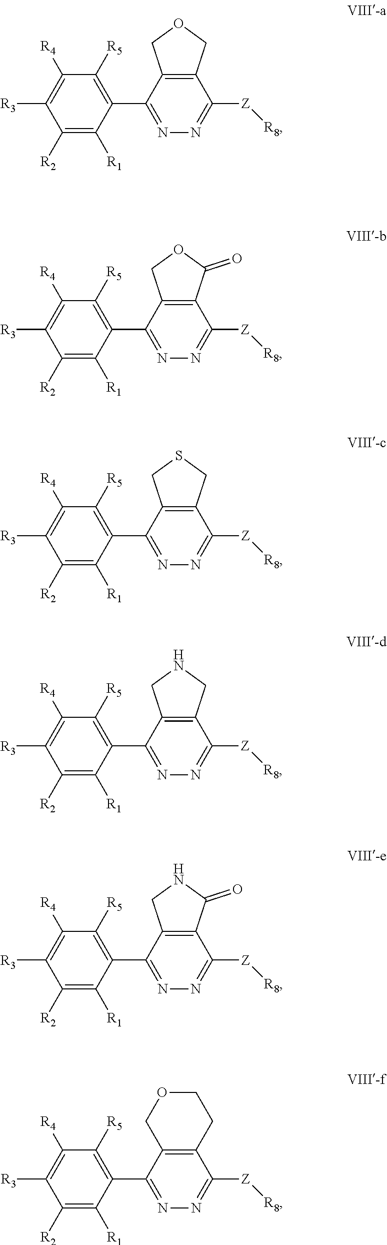

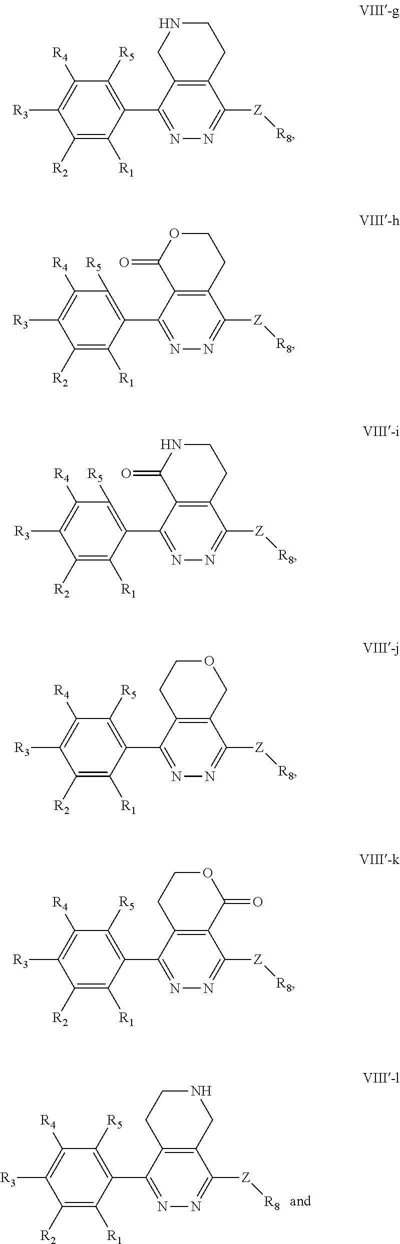

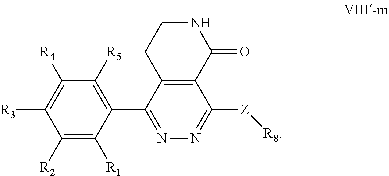

In some embodiments, the compound of formula I is selected from the group consisting of compounds of formulas VIII′-a, VIII′-c, VIII′-d, VIII′-f, VIII′-g, VIII′-j, VIII′-k, VIII′-l and VIII′-m, preferably compounds of formulas VIII′-a, VIII′-c, VIII′-d, VIII′-f, VIII′-g, VIII′-j and VIII′-l, and more preferably compounds of formulas VIII′-a, VIII′-c, VIII′-d and VIII′-j, and is most preferably a compound of formula VIII′-a or VIII′-d.

In some embodiments, R₆ and R₇, together with the atoms to which they are attached, form phenyl that is optionally substituted with one or more substituents selected from the group consisting of: deuterium, halogen, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, the compound of formula I is selected from the group consisting of:

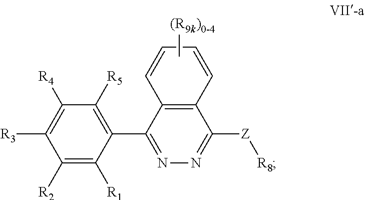

R₉ₖ is independently selected from the group consisting of deuterium, halogen, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
preferably, R₉ₖ is independently selected from the group consisting of deuterium, halogen, oxo, C₁₋₆ alkyl and —O—C₁₋₆ alkyl. In some embodiments, in the compounds of formulas VII-a to VII-k, VII′-a, formulas VIII-a to VIII-g and formulas VIII′-a to VIII′-m, R₂, R₃, R₄ and R₅ are independently selected from the group consisting of hydrogen, deuterium, halogen, and the following groups that are optionally substituted with one or more substituents: C₁₋₆ alkyl, C₃₋₆ cycloalkyl and C₃₋₆ cycloalkylmethylene, and the substituents are deuterium atoms or halogens; more preferably, R₉ₖ is independently selected from the group consisting of deuterium, halogen, oxo, C₁₋₆ haloalkyl and —O—C₁₋₆ haloalkyl; most preferably, R₉ₖ is independently selected from the group consisting of deuterium, halogen, C₁₋₆ alkyl and —O—C₁₋₆ alkyl.

In some embodiments, R₂ and R₄ are independently selected from the group consisting of hydrogen, deuterium and halogen, and R₃ and R₅ are independently selected from the group consisting of hydrogen, deuterium, halogen, and C₁₋₆ alkyl that is optionally substituted with one or more deuterium atoms or halogens.

In some embodiments, R₂ and R₄ are each hydrogen, R₃ is trifluoromethyl or chlorine, and R₅ is hydrogen, halogen or methyl; preferably, R₂, R₄ and R₅ are each hydrogen, and R₃ is trifluoromethyl.

In some embodiments, R₆ and R₇, together with the atoms to which they are attached, form 5-6 membered heteroaryl that is optionally substituted with one or more substituents selected from the group consisting of: deuterium, halogen, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; preferably, the 5-6 membered heteroaryl is pyridine;
R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

In some embodiments, R₆ and R₇, together with the atoms to which they are attached, form 5-6 membered heteroaryl that contains 1-2 heteroatoms and is optionally substituted with one or more substituents selected from the group consisting of: deuterium, halogen, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens; the heteroatoms are selected from the group consisting of oxygen, nitrogen and sulfur atoms, preferably from nitrogen atoms.

In some embodiments, the compound of formula I is selected from the group consisting of:

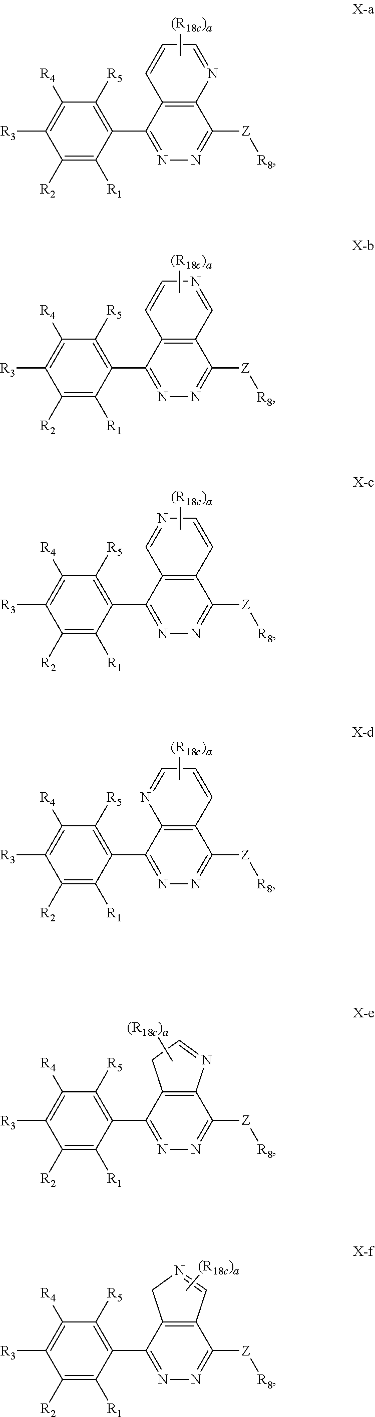

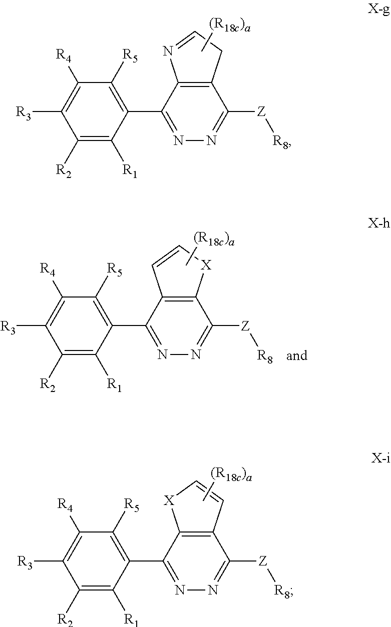

X is selected from the group consisting of an oxygen atom and a sulfur atom; the R_{18c} is selected from the group consisting of deuterium, halogen, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more halogens; preferably, R_{18c}, is independently selected from the group consisting of deuterium, halogen, C₁₋₆ alkyl and —O—C₁₋₆ alkyl;
a is an integer selected from 0-3; preferably, a is an integer selected from 0-1; more preferably, a is 0;
Z, R₂, R₃, R₄, R₅ and R₈ are as defined in formula I.

In some embodiments, in the compounds of formulas X-a, X-b, X-c, X-d, X-e, X-f, X-g, X-h and X-i or the pharmaceutically acceptable salts thereof, R_{18c}, is independently selected from the group consisting of deuterium, halogen, C₁₋₆ alkyl and —O—C₁₋₆ alkyl.

In some embodiments, in the compounds of formulas X-a, X-b, X-c, X-d, X-e, X-f, X-g, X-h and X-i or the pharmaceutically acceptable salts thereof, R₂, R₃, R₄ and R₅ are independently selected from the group consisting of hydrogen, deuterium, halogen, and the following groups that are optionally substituted with one or more substituents: C₁₋₆ alkyl, C₃₋₆ cycloalkyl and C₃₋₆ cycloalkylmethylene, and the substituents are deuterium atoms or halogens.

In some embodiments, in the compounds of formulas X-a, X-b, X-c, X-d, X-e, X-f, X-g, X-h and X-i or the pharmaceutically acceptable salts thereof, R₂ and R₄ are independently selected from the group consisting of hydrogen, deuterium and halogen, and R₃ and R₅ are independently selected from the group consisting of hydrogen, deuterium, halogen, and C₁₋₆ alkyl that is optionally substituted with one or more deuterium atoms or halogens. In some embodiments, in the compounds of formulas X-a, X-b, X-c, X-d, X-e, X-f, X-g, X-h and X-i or the pharmaceutically acceptable salts thereof, R₂ and R₄ are each hydrogen, R₃ is trifluoromethyl or chlorine, and R₅ is hydrogen or methyl; preferably, R₂, R₄ and R₅ are each hydrogen, and R₃ is trifluoromethyl.

The present disclosure further provides a compound of formula I′ or a pharmaceutically acceptable salt thereof,

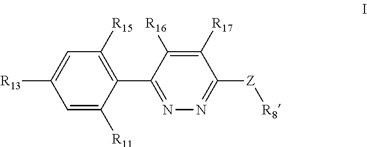

wherein R₁₁ is selected from the group consisting of —OH, —NH₂, —CN, —O—C₁₋₆ alkyl, —C₁₋₆ alkyl-OH and —NHC(═O)—C₁₋₆ alkyl, and the C₁₋₆ alkyl is optionally substituted with one or more substituents selected from the group consisting of: deuterium, halogen, —OH, —NH₂ and —CN;
R₁₃ is selected from the group consisting of the following groups that are optionally substituted with one or more substituents: C₂₋₆ alkyl, —S—C₁₋₆ alkyl and C₃₋₆ cycloalkyl, and the substituents are selected from the group consisting of deuterium, halogen and OH.
R₁₅, R₁₆ and R₁₇ are independently selected from the group consisting of hydrogen, deuterium, halogen, —OH, —NH₂ and C₁₋₆ alkyl, and the C₁₋₆ alkyl is optionally substituted with one or more substituents selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR_{8b}S(═O)₂R_{8b}, —S(═O)₂R_{8b}, —S(═O) NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

In some embodiments, R₁₃ is selected from the group consisting of ethyl, n-propyl, isopropyl and n-butyl, and is preferably ethyl;

In one embodiment, R₁₃ is —S—CF₃.

In some embodiments, R₁₃ is selected from the group consisting of:

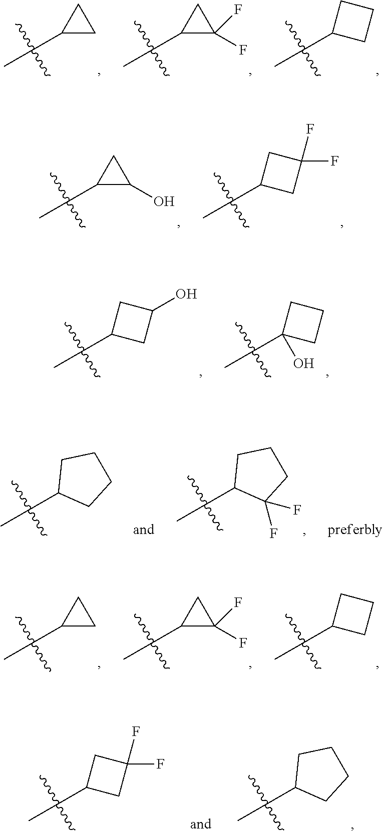

and more preferably

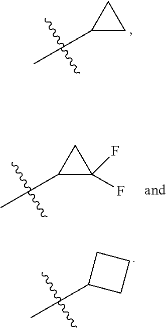

and is most preferably

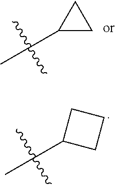

In one embodiment, R₁₁ is —OH.

In one embodiment, R₁₁ is —NH₂.

In some embodiments, R₁₁ is —O—C₁₋₆ alkyl that is optionally substituted with a deuterium atom or a halogen; preferably, R₁₁ is methoxy or ethoxy; more preferably, R₁₁ is methoxy.

In one embodiment, R₁₁ is —CH₂OH.

In some embodiments, R₁₁ is —NHC(═O)—C₁₋₆ alkyl that is optionally substituted with a deuterium atom or a halogen; preferably, R₁₁ is —NHC(═O)—CH₃.

In some embodiments, R₁₅, R₁₆ and R₁₇ are independently selected from the group consisting of hydrogen, deuterium, fluorine and methyl.

The present disclosure further provides a compound of formula I″ or a pharmaceutically acceptable salt thereof,

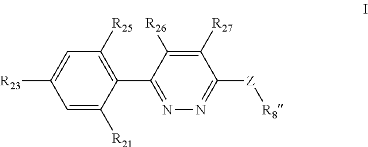

wherein R₂₁ is selected from the group consisting of —NH₂, —O—C₁₋₆ alkyl, —C₁₋₆ alkyl-OH and —NHC(═O)—C₁₋₆ alkyl, and the C₁₋₆ alkyl is optionally substituted with one or more substituents selected from the group consisting of: deuterium, halogen, —OH, —NH₂ and —CN;
R₂₃ is selected from the group consisting of chlorine and C₁₋₆ alkyl that is optionally substituted with one or more deuterium atoms or halogens;
R₂₅, R₂₆ and R₂₇ are independently selected from the group consisting of hydrogen, deuterium, halogen, —OH, —NH₂ and C₁₋₆ alkyl, and the C₁₋₆ alkyl is optionally substituted with one or more substituents selected from the group consisting of: deuterium, halogen, —OH, —NH₂ and —CN;
Z is O or —NH—(CH₂)n-, and n is an integer selected from 0-3;
R₈ is selected from the group consisting of aryl, heteroaryl, heterocyclyl, C₃₋₈ cycloalkyl, C₁₋₆ alkyl and —O—C₁₋₆ alkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O) NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens; and when R₂₃ is methyl, R₂₁ is not methoxy.

In one embodiment, R₂₁ is —NH₂.

In some embodiments, R₂₁ is —O—C₁₋₆ alkyl that is optionally substituted with a deuterium atom or a halogen; preferably, R₂₁ is methoxy or ethoxy; more preferably, R₂₁ is methoxy.

In one embodiment, R₂₁ is —CH₂OH.

In some embodiments, R₂₁ is —NHC(═O)—C₁₋₆ alkyl that is optionally substituted with a deuterium atom or a halogen; preferably, R₂₁ is —NHC(═O)—CH₃.

In some embodiments, R₂₃ is C₁₋₆ alkyl that is optionally substituted with one or more fluorine atoms; preferably, R₂₃ is trifluoromethyl.

In one embodiment, R₂₃ is chlorine.

In some embodiments, R₂₅, R₂₆ and R²⁷ are independently selected from the group consisting of hydrogen, deuterium, fluorine and methyl.

In some embodiments, in the aforementioned compounds of the present disclosure, Z is O.

In some embodiments, in the aforementioned compounds of the present disclosure, Z is —NH—(CH₂)n-, and n is an integer selected from 0-2; n is preferably 0 or 1; n is more preferably 0.

In some embodiments, in the aforementioned compounds of the present disclosure, R₈ is selected from 5-10 membered heterocyclyl that is optionally substituted with one or more substituents selected from the group consisting of deuterium, halogen, oxo, —OR₈ₐ, —SR₈ₐ, —C(O)R₈ₐ, —OC(═O)R₈ₐ, —C(═O)OR₈ₐ, —C(O)NR₈ₐR_{8b}, —NR₈ₐR_{8b}, —NR₈ₐC(═O)R_{8b}, —NR₈ₐS(═O)₂R_{8b}, —S(═O)₂R_{8b}, —S(═O)₂NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

In some R₈ is selected from the group consisting of:

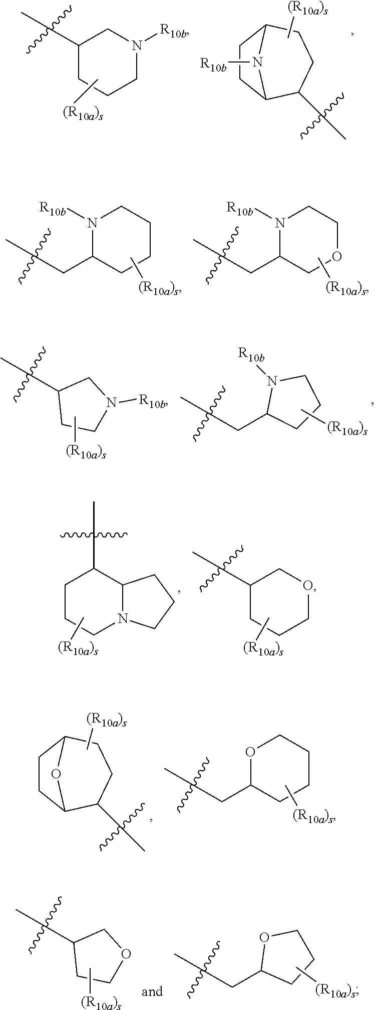

R₁₀ₐ is selected from the group consisting of hydrogen, deuterium, halogen, oxo, —OH, —NH₂, —COOH, —CN, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R_{10b} is selected from the group consisting of hydrogen, deuterium, C₁₋₄ alkyl and C₃₋₆ cycloalkyl, and the C₁₋₄ alkyl and C₃₋₆ cycloalkyl are optionally further substituted with one or more deuterium atoms or halogens; s is an integer selected from 0-15.

In some embodiments, R₈ is selected from the group consisting of:

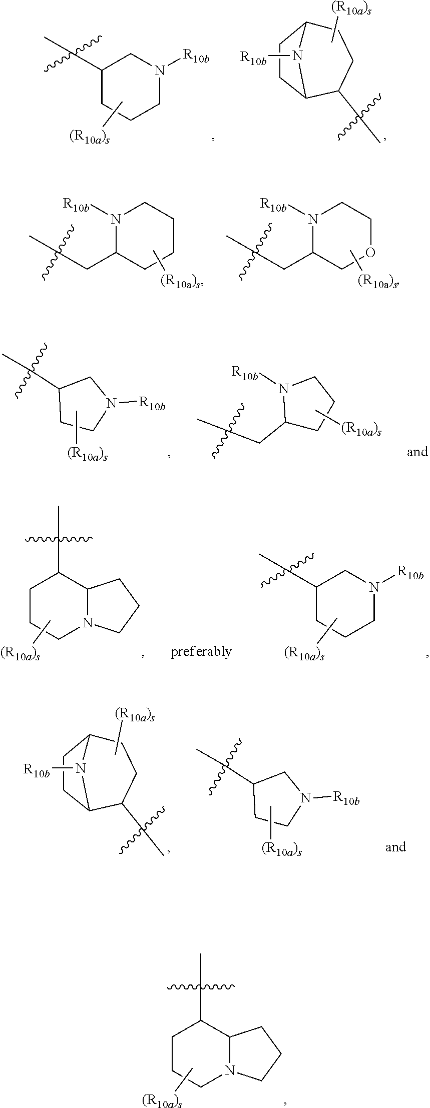

and is more preferably

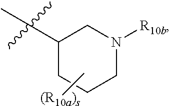

and most preferably

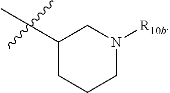

In some embodiments, R₈ is

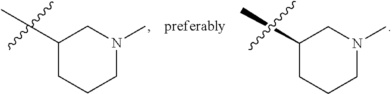

In some other embodiments, in the aforementioned compounds of the present disclosure, R₈ is selected from the group consisting of aryl and heteroaryl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O)₂NR₈ₐ,R_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

In some embodiments, R₈ is selected from the group consisting of:

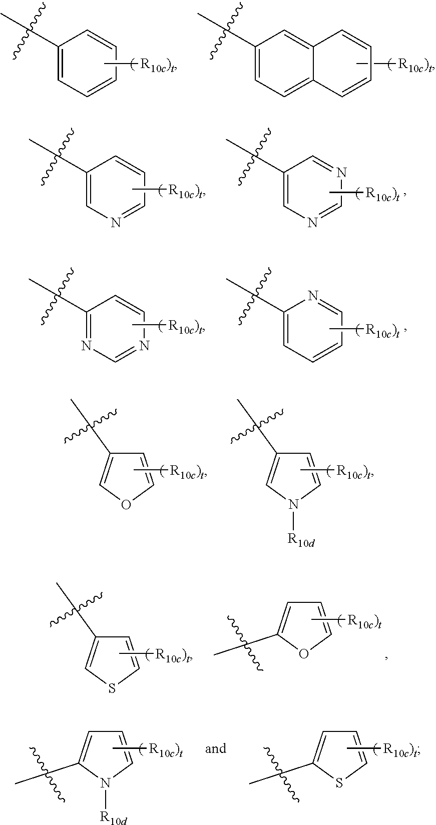

R_{10c} is selected from the group consisting of hydrogen, deuterium, halogen, —OH, —NH₂, —COOH, —CN, —C(O)₂NH₂, —S(═O)₂NH₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R_{10d} is selected from the group consisting of hydrogen, deuterium, C₁₋₄ alkyl and C₃₋₆ cycloalkyl, and the C₁₋₄ alkyl and C₃₋₆ cycloalkyl are optionally further substituted with one or more deuterium atoms or halogens; t is an integer selected from 0-7.

In some embodiments, R₈ is selected from the group consisting of:

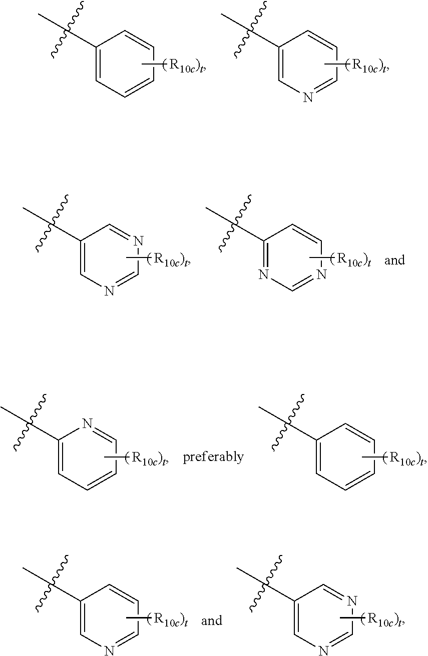

and is more preferably

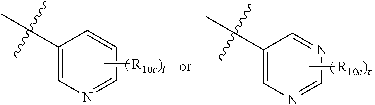

In some other embodiments, in the aforementioned compounds of the present disclosure, R₈ is selected from C₃₋₈ cycloalkyl that is optionally substituted with one or more substituents selected from the group consisting of deuterium, halogen, —OH, —NH₂, —CN and C₁₋₄ alkyl, and the C₁₋₄ alkyl is optionally further substituted with one or more deuterium atoms, halogens or —OH.

In some embodiments, R₈ is selected from the group consisting of:

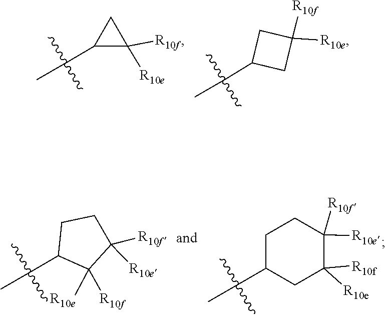

R₁₀ₑ, R_{10f}, R_{10e′} and R_{10f′} are independently selected from the group consisting of hydrogen, deuterium, halogen, —OH and C₁₋₄ alkyl, and the C₁₋₄ alkyl is optionally further substituted with one or more deuterium atoms, halogens or —OH.

In some embodiments, R₈ is selected from the group consisting if

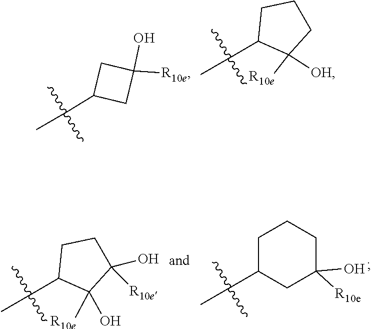

R₁₀ₑ and R_{10 e′} are independently selected from the group consisting of hydrogen, deuterium, halogen, —OH and C₁₋₄ alkyl, and the C₁₋₄ alkyl is optionally further substituted with one or more deuterium atoms, halogens or —OH.

In some embodiments, R₈ is selected from the group consisting of:

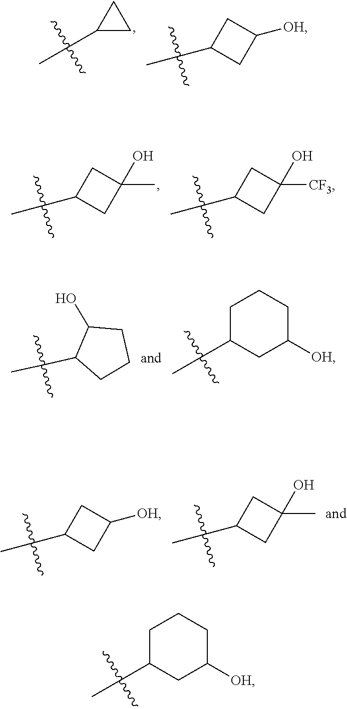

and is more preferably

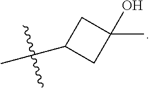 [SMILES: *C1CC(C)(O)C1]

In some other embodiments, in the aforementioned compounds of the present disclosure, R₈ is selected from C₂₋₆ alkyl that is optionally substituted with one or more substituents selected from the group consisting of deuterium, halogen, —OR₈ₐ, —SR₈ₐ, —C(═O)R₈ₐ, —OC(═O)R₈ₐ, —C(═O)OR₈ₐ, —C(O)NR₈ₐR_{8b}, —NR₈ₐR_{8b}, —NR₈ₐC(O)R_{8b}, —NR₈ₐS(═O)₂R_{8b}, —S(═O)₂R₈ₐ, —S(═O) NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

In some embodiments, R₈ is selected from the group consisting of:

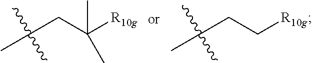

R_{10g} is selected from the group consisting of hydrogen, deuterium, halogen, —NH₂, —OH, —CN, —NH—C₁₋₄ alkyl and —N(C₁₋₄ alkyl)₂, and the C₁₋₄ alkyl is optionally substituted with one or more deuterium atoms, halogens or —OH.

In a second aspect, the present disclosure provides a compound or a pharmaceutically salt thereof selected from the group consisting of

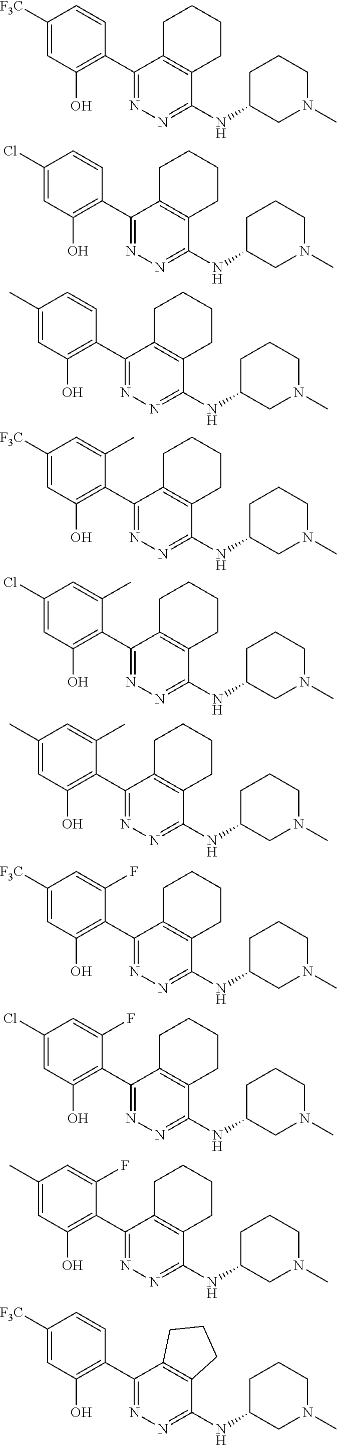

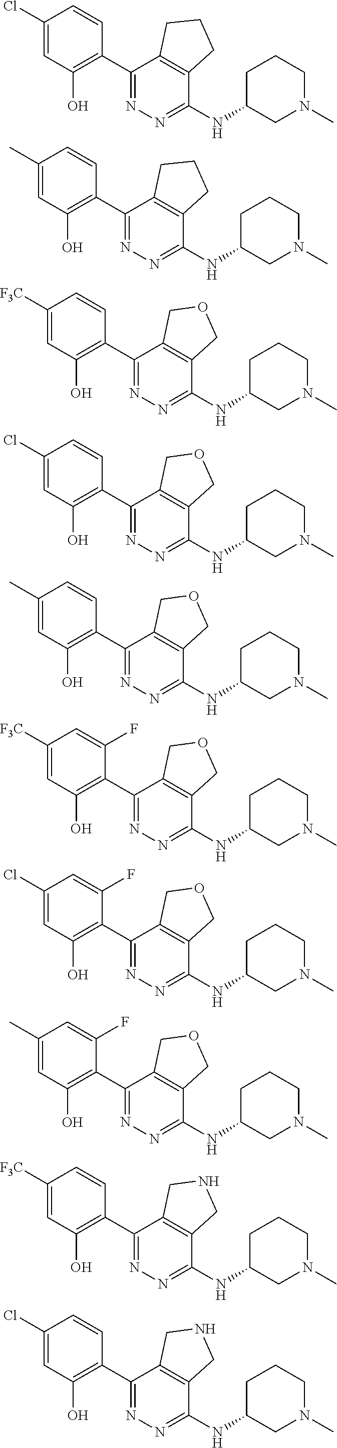

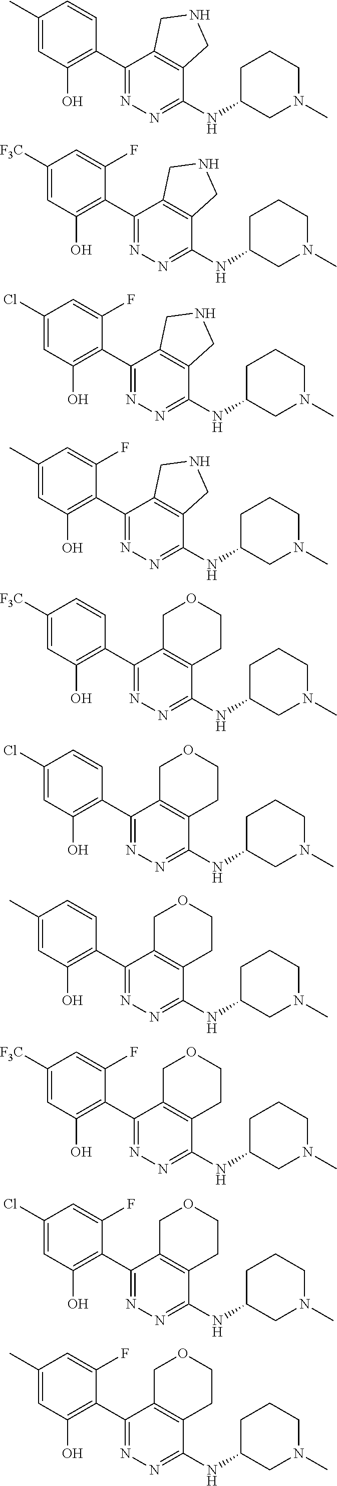

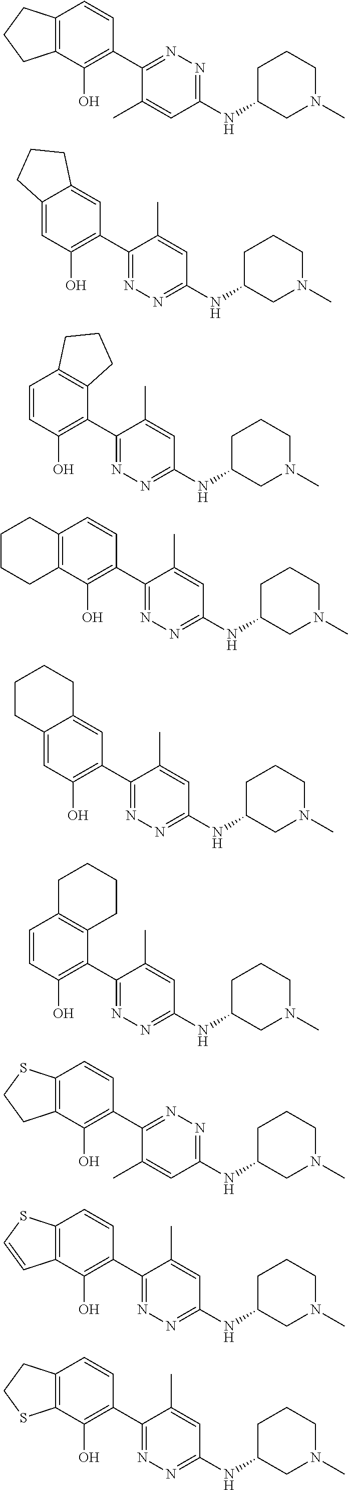

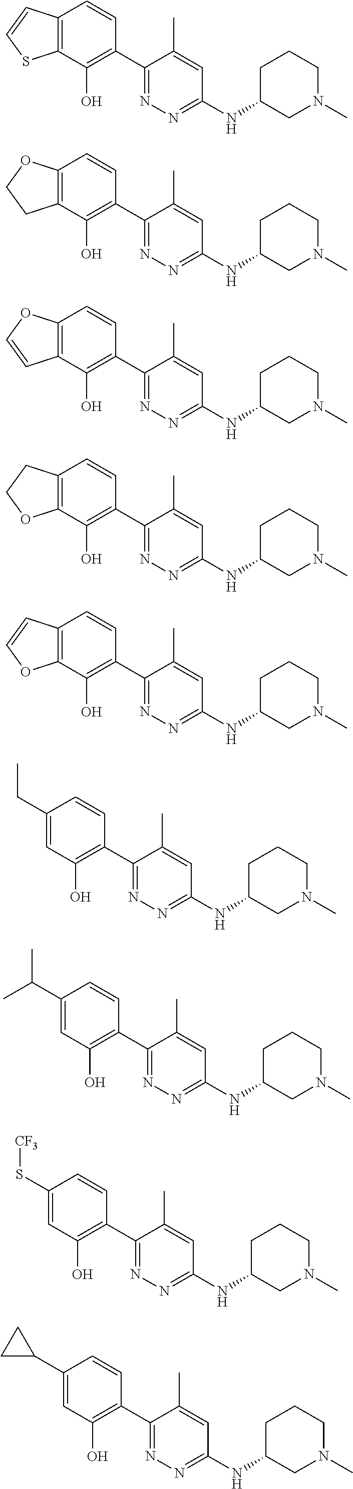

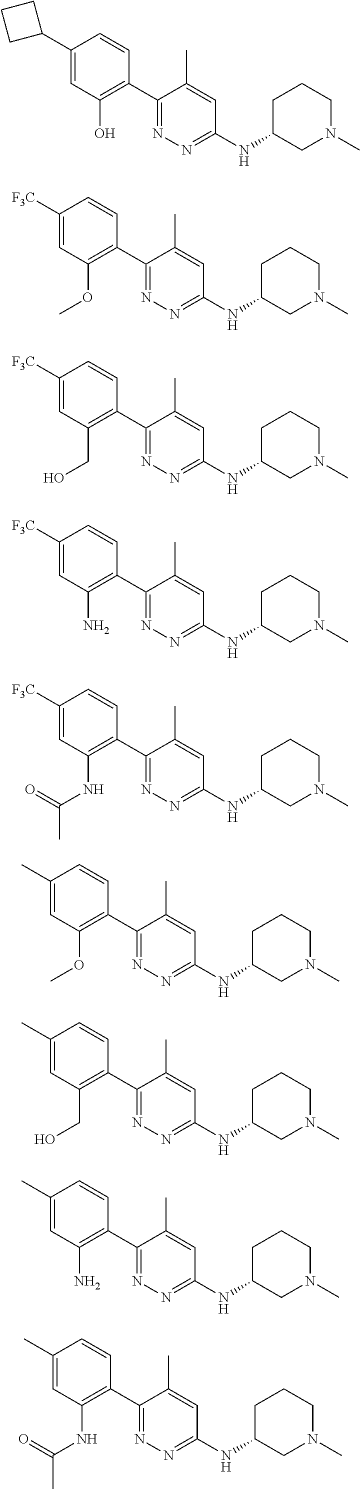

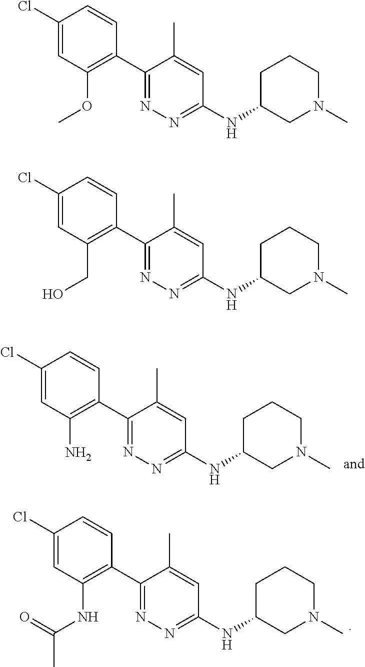 [SMILES: COc1cc(Cl)ccc1-c1nnc(N[C@@H]2CCCN(C)C2)cc1C ; Cc1cc(N[C@@H]2CCCN(C)C2)nnc1-c1ccc(Cl)cc1CO ; Cc1cc(N[C@@H]2CCCN(C)C2)nnc1-c1ccc(Cl)cc1N ; CC(=O)Nc1cc(Cl)ccc1-c1nnc(N[C@@H]2CCCN(C)C2)cc1C]

In a third aspect, the present disclosure provides a compound or a pharmaceutically acceptable salt thereof selected from the group consisting of:

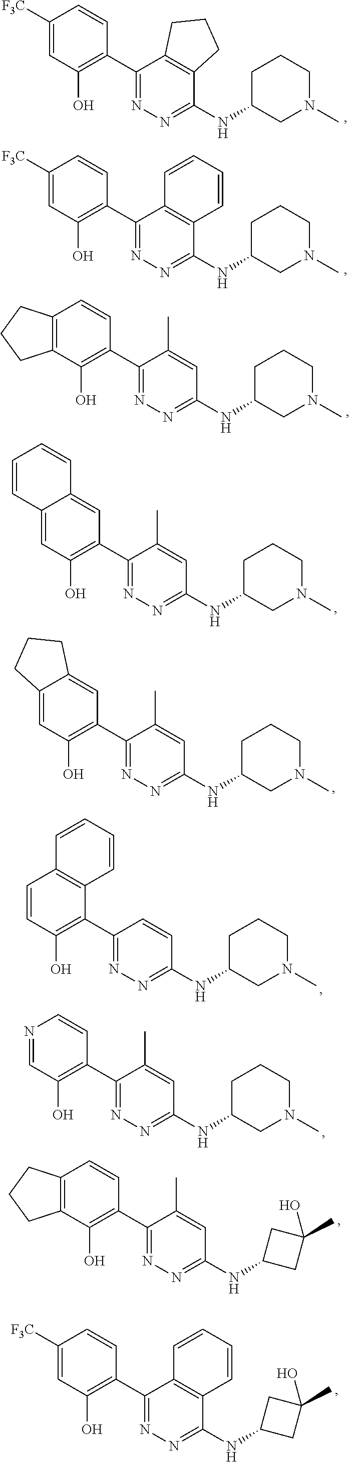

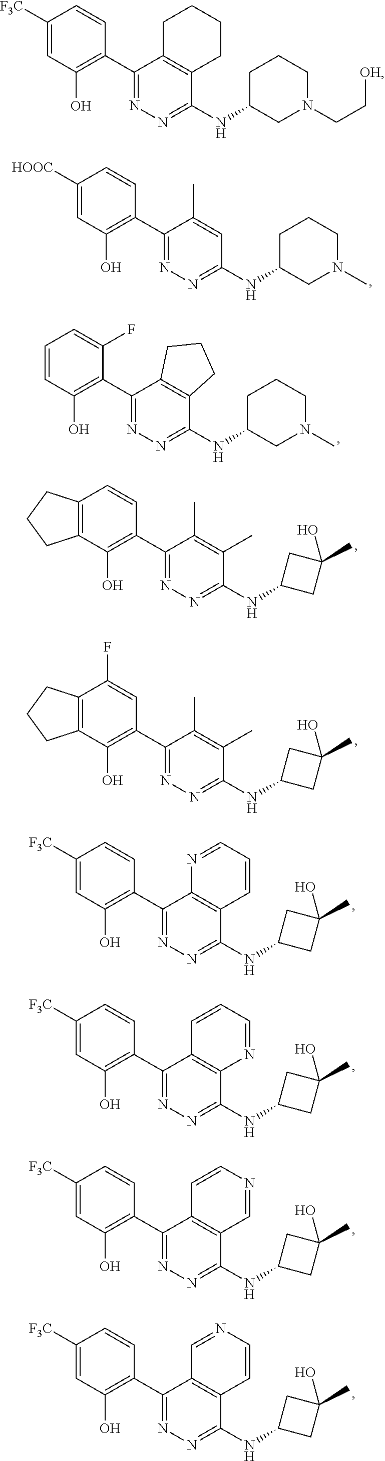

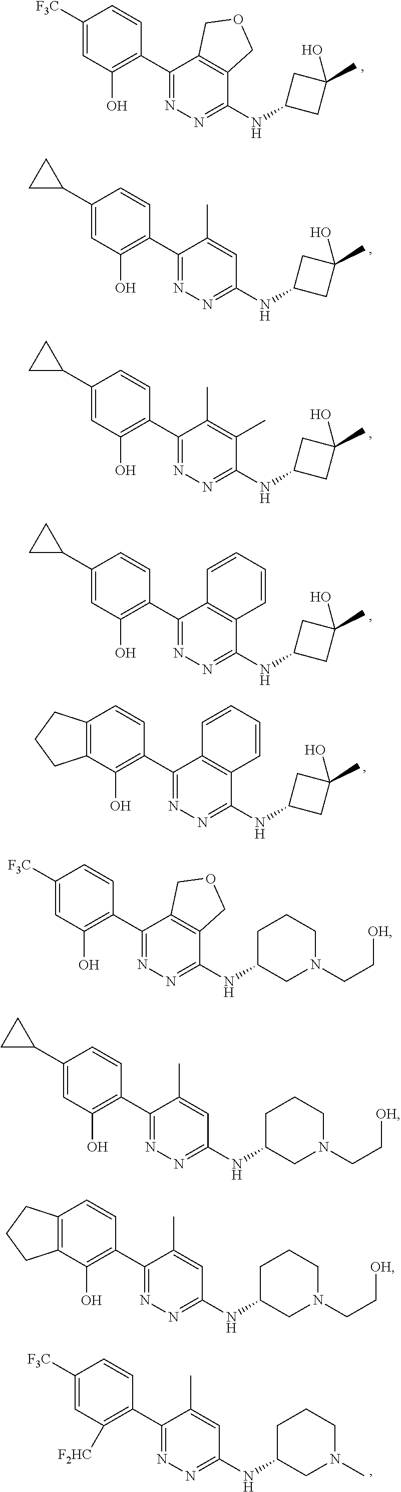

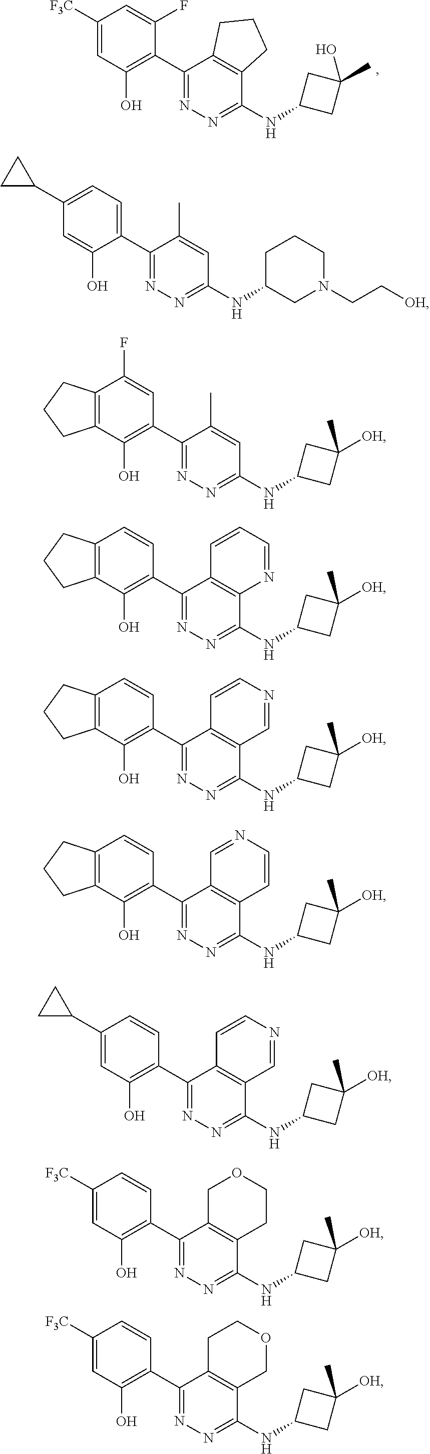

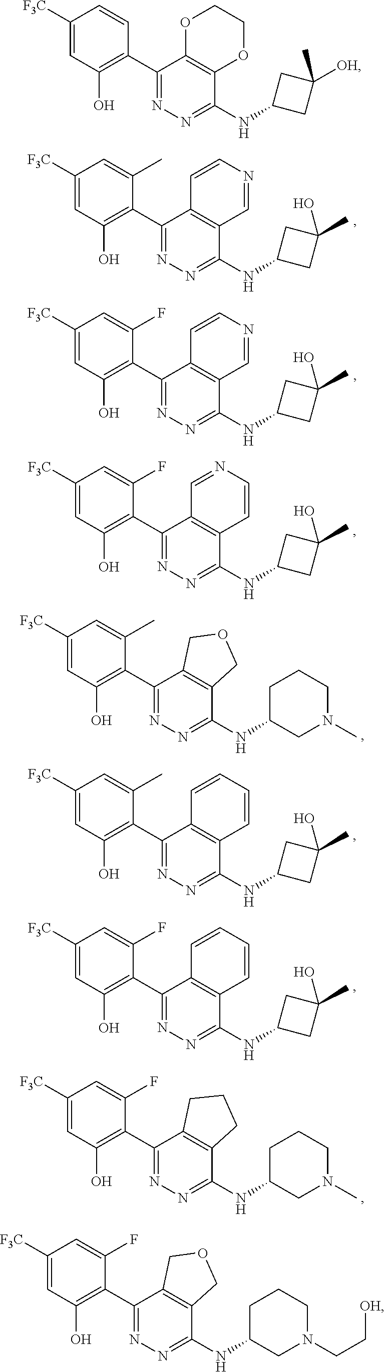

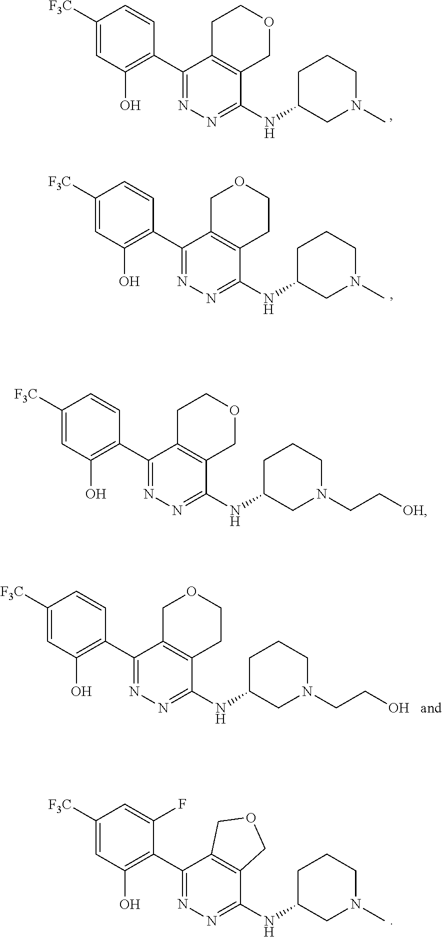 [SMILES: CN1CCC[C@@H](Nc2nnc(-c3ccc(C(F)(F)F)cc3O)c3c2COCC3)C1 ; CN1CCC[C@@H](Nc2nnc(-c3ccc(C(F)(F)F)cc3O)c3c2CCOC3)C1 ; OCCN1CCC[C@@H](Nc2nnc(-c3ccc(C(F)(F)F)cc3O)c3c2COCC3)C1 ; OCCN1CCC[C@@H](Nc2nnc(-c3ccc(C(F)(F)F)cc3O)c3c2CCOC3)C1 ; CN1CCC[C@@H](Nc2nnc(-c3c(O)cc(C(F)(F)F)cc3F)c3c2COC3)C1]

In a fourth aspect, the present disclosure further provides an isotopically substituted form of the aforementioned compound; preferably, the isotopic substitution is a substitution with a deuterium atom.

In a fifth aspect, the present disclosure further provides a pharmaceutical composition comprising the compound or the pharmaceutically acceptable salt thereof according to the first to third aspects or the isotopically substituted form according to the forth aspect, and at least one pharmaceutically acceptable carrier, diluent or excipient.

In some embodiments, a unit dose of the pharmaceutical composition is 0.001 mg-1000 mg.

In certain embodiments, the pharmaceutical composition comprises 0.01-99.99% of the aforementioned compound or pharmaceutically acceptable salt thereof on the basis of the total weight of the composition. In certain embodiments, the pharmaceutical composition comprises 0.1-99.9% of the aforementioned compound or pharmaceutically acceptable salt thereof. In certain embodiments, the pharmaceutical composition comprises 0.5% 99.5% of the compound or the pharmaceutically acceptable salt thereof. In certain embodiments, the pharmaceutical composition comprises 1%-99% of the compound or the pharmaceutically acceptable salt thereof. In certain embodiments, the pharmaceutical composition comprises 2%-98% of the compound or the pharmaceutically acceptable salt thereof.

The present disclosure further provides use of the compound or the pharmaceutically acceptable salt thereof according to the first, second or third aspect as a medicament. In a sixth aspect, the present disclosure further provides use of the compound or the pharmaceutically acceptable salt thereof according to the first, second or third aspect, the isotopically substituted form according to the fourth aspect or the pharmaceutical composition according to the fifth aspect in the preparation of a medicament for treating a disease associated with NLRP3 activity.

The present disclosure further provides a method for preventing and/or treating a disease associated with NLRP3 activity in a patient, which comprises administering to the patient a therapeutically effective amount of the compound or the pharmaceutically acceptable salt thereof according to the first, second or third aspect, the isotopically substituted form according to the fourth aspect or the pharmaceutical composition according to the fifth aspect.

The present disclosure further provides the compound or the pharmaceutically acceptable salt thereof according to the first, second or third aspect, the isotopically substituted form according to the fourth aspect or the pharmaceutical composition according to the fifth aspect for use in the prevention or treatment of a disease associated with NLRP3 activity. The present disclosure further provides a method for preventing and/or treating a disease associated with NLRP3 activity in a patient, which comprises administering to the patient a therapeutically effective amount of the aforementioned compound or pharmaceutically acceptable salt thereof, or the aforementioned pharmaceutical composition.

The disease associated with NLRP3 activity includes inflammasome-related diseases, immune diseases, inflammatory diseases, autoimmune diseases and/or autoinflammatory diseases.

The present disclosure further provides use of the compound or the pharmaceutically acceptable salt thereof according to the first, second or third aspect, the isotopically substituted form according to the fourth aspect or the pharmaceutical composition according to the fifth aspect in the preparation of a medicament for treating inflammasome-related diseases, immune diseases, inflammatory diseases, autoimmune diseases and/or autoinflammatory diseases.

The present disclosure further provides use of the compound or the pharmaceutically acceptable salt thereof according to the first, second or third aspect, the isotopically substituted form according to the fourth aspect or the pharmaceutical composition according to the fifth aspect in the preparation of a medicament for treating inflammasome-related diseases, immune diseases, inflammatory diseases, autoimmune diseases and/or autoinflammatory diseases.

The present disclosure further provides the compound or the pharmaceutically acceptable salt thereof according to the first, second or third aspect, the isotopically substituted form according to the fourth aspect or the pharmaceutical composition according to the fifth aspect for use in the treatment of inflammasome-related diseases, immune diseases, inflammatory diseases, autoimmune diseases and/or autoinflammatory diseases.

The present disclosure further provides a method for treating and/or preventing an inflammasome-related disease, an immune disease, an inflammatory disease, an autoimmune disease and/or an autoinflammatory disease in a patient, which comprises administering to the patient a therapeutically effective amount of the aforementioned compound or pharmaceutically acceptable salt thereof, or the aforementioned pharmaceutical composition. The inflammasome-related disease, the immune disease, the inflammatory disease, the autoimmune disease and/or the autoinflammatory disease may be specifically selected from the group consisting of: autoinflammatory fever syndromes (such as cryo-pyrin-associated periodic syndromes), sickle-cell anemia, systemic lupus erythematosus, liver-related diseases (such as chronic liver disease, viral hepatitis, nonalcoholic steatohepatitis, alcoholic steatohepatitis and alcoholic liver disease), inflammatory arthritis-related diseases (such as gout, chondrocalcinosis, osteoarthritis, rheumatoid arthritis and acute or chronic arthritis), kidney-related diseases (such as hyperoxaluria, lupus nephritis, hypertensive nephropathy, hemodialysis-associated inflammation, type I or type II diabetes and complications thereof (such as nephropathy and retinopathy)), neuroinflammation-related diseases (such as brain infection, acute injury, multiple sclerosis, Alzheimer's disease and neurodegenerative diseases), cardiovascular and metabolic disorders or diseases (such as cardiovascular risk reduction (CvRR), atherosclerosis, type I and type II diabetes and related complications, peripheral artery disease (PAD), acute heart failure and hypertension), wound healing, scar formation, inflammatory skin diseases (e.g., acne and hidradenitis suppurativa), asthma, sarcoidosis, age-related macular degeneration, and cancer-related diseases/conditions (e.g., myeloproliferative neoplasms, leukemias, myelodysplastic syndromes (MDS), myelofibrosis, lung cancer and colon cancer).

In some embodiments, the AUC or Cₘₐₓ in the blood after oral administration of the compounds of the present disclosure compared to compound R1 or compound R2 or compound R3 is expected to be increased by 10%, 20%, 30%, 40%, 50%, 60%, 70%, 80%, 90%, 100% or even higher.

The present disclosure further provides a compound as shown below,

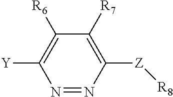

wherein Y is selected from the group consisting of fluorine, chlorine, bromine and iodine; preferably, Y is selected from the group consisting of fluorine, chlorine and bromine; more preferably, Y is selected from chlorine;
R₆, R₇, Z and R₈ are as defined in formula I.

In certain embodiments, the compound is an intermediate.

In some embodiments, R₆ and R₇, together with the atoms to which they are attached, form a 5-6 membered cyclic hydrocarbon, a 5-6 membered heterocyclic ring, phenyl or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₂, R₃, R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

The pharmaceutically acceptable salts of the compounds described herein are selected from the group consisting of inorganic salts and organic salts. The compounds described herein can react with acidic or basic substances to form corresponding salts.

In another aspect, the compounds of the present disclosure may exist in specific step isomeric forms. The present disclosure contemplates all such compounds, including cis and trans isomers, (−)- and (+)-enantiomers, (R)- and (S)-enantiomers, diastereomers, (D)-isomer, (L)-isomer, and racemic mixtures and other mixtures thereof, such as enantiomerically or diastereomerically enriched mixtures, all of which are within the scope of the present disclosure. Additional asymmetric carbon atoms may be present in substituents such as an alkyl group. All such isomers and mixtures thereof are included within the scope of the present disclosure.

The compounds and intermediates of the present disclosure may also exist in different tautomeric forms, and all such forms are included within the scope of the present disclosure. The term “tautomer” or “tautomeric form” refers to structural isomers of different energies that can interconvert via a low energy barrier. For example, proton tautomers (also known as proton transfer tautomers) include interconversion via proton migration, such as keto-enol and imine-enamine, lactam-lactim isomerization. An example of a lactam-lactim equilibrium is present between A and B as shown below.

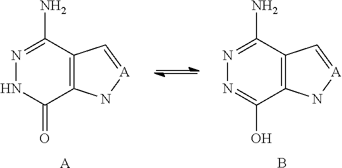

All the compounds in the present invention can be drawn as form A or form B. All tautomeric forms fall within the scope of the present invention. The nomenclature of the compounds does not exclude any tautomers.

The compounds of the present disclosure may be asymmetric; for example, the compounds have one or more stereoisomers. Unless otherwise specified, all stereoisomers include, for example, enantiomers and diastereomers. The compounds of the present disclosure containing asymmetric carbon atoms can be isolated in optically active pure form or in racemic form. The optically active pure form can be isolated from a racemic mixture or synthesized using chiral starting materials or chiral reagents.

Optically active (R)- and (S)-enantiomers, and D- and L-isomers can be prepared by chiral synthesis, chiral reagents or other conventional techniques. If one enantiomer of a certain compound of the present disclosure is desired, it may be prepared by asymmetric synthesis or derivatization with a chiral auxiliary, wherein the resulting mixture of diastereomers is separated and the auxiliary group is cleaved to provide the pure desired enantiomer. Alternatively, when the molecule contains a basic functional group (e.g., amino) or an acidic functional group (e.g., carboxyl), salts of diastereomers are formed with an appropriate optically active acid or base, followed by resolution of diastereomers by conventional methods known in the art, and the pure enantiomers are obtained by recovery. Furthermore, separation of enantiomers and diastereomers is typically accomplished by chromatography using a chiral stationary phase, optionally in combination with chemical derivatization (e.g., carbamate formation from amines).

The present disclosure also comprises isotopically-labeled compounds which are identical to those recited herein but have one or more atoms replaced by an atom having an atomic mass or mass number different from the atomic mass or mass number usually found in nature. Examples of isotopes that can be incorporated into the compound of the present disclosure include isotopes of hydrogen, carbon, nitrogen, oxygen, phosphorus, sulfur, fluorine, iodine, and chlorine, such as ²H, ³H, ¹¹C, ¹³C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl.

Unless otherwise specified, when a position is specifically assigned deuterium (D), the position should be construed as deuterium with an abundance that is at least 1000 times greater than the natural abundance of deuterium (which is 0.015%) (i.e., at least 10% deuterium incorporation). The compounds of examples comprise deuterium having an abundance that is greater than at least 1000 times the natural abundance, at least 2000 times the natural abundance, at least 3000 times the natural abundance, at least 4000 times the natural abundance, at least 5000 times the natural abundance, at least 6000 times the natural abundance, or higher times the natural abundance. The present disclosure also comprises various deuterated forms of the compound of formula (I). Each available hydrogen atom connected to a carbon atom may be independently replaced with a deuterium atom. Those skilled in the art are able to synthesize the deuterated forms of the compound of general formula (I) with reference to the relevant literature. Commercially available deuterated starting materials can be used in preparing the deuterated forms of the compound of formula (I), or they can be synthesized using conventional techniques with deuterated reagents including, but not limited to, deuterated borane, tri-deuterated borane in tetrahydrofuran, deuterated lithium aluminum hydride, deuterated iodoethane, deuterated iodomethane, and the like.

“Optionally” or “optional” means that the event or circumstance subsequently described may, but does not necessarily, occur and that the description includes instances where the event or circumstance occurs or does not occur. For example, “C₁₋₆ alkyl that is optionally substituted with a halogen or cyano” means that the halogen or cyano may, but does not necessarily, exist, and the description includes the instance where alkyl is substituted with a halogen or cyano and the instance where alkyl is not substituted with a halogen and cyano.

In the chemical structure of the compound of the present invention, a bond [symbol] represents an unspecified configuration—that is, if chiral isomers exist in the chemical structure, the bond “ [symbol] ” may be “ [symbol] ” or “ [symbol] ”, or includes both the configurations of “ [symbol] ” and “ [symbol] ”. Although all of the above structural formulae are drawn as certain isomeric forms for the sake of simplicity, the present disclosure may include all isomers, such as tautomers, rotamers, geometric isomers, diastereomers, racemates and enantiomers.

### Terms and Definitions:

“Pharmaceutical composition” refers to a mixture containing one or more of the compounds or the physiologically/pharmaceutically acceptable salts or pro-drugs thereof described herein, and other chemical components, for example, physiologically/pharmaceutically acceptable carriers and excipients. The pharmaceutical composition is intended to promote the administration to an organism, so as to facilitate the absorption of the active ingredient, thereby exerting biological activities.

“Pharmaceutically acceptable excipient” includes, but is not limited to, any adjuvant, carrier, excipient, glidant, sweetener, diluent, preservative, dye/colorant, flavoring agent, surfactant, wetting agent, dispersant, suspending agent, stabilizer, isotonic agent, solvent or emulsifier that has been approved by the U.S. food and drug administration as acceptable for use in humans or livestock animals.

“Alkyl” refers to a saturated aliphatic hydrocarbon group, including linear and branched groups of 1 to 20 carbon atoms, and alkyl containing 1 to 6 carbon atoms. Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, various branched isomers thereof, and the like. Unless otherwise specified, alkyl may be substituted or unsubstituted, and when it is substituted, the substituent may be substituted at any accessible connection site, preferably with one or more groups independently selected from the group consisting of halogen, deuterium, hydroxy, oxo, nitro, cyano, C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₈ cycloalkenyloxy, C₆₋₁₀ aryl or 5- to 6-membered heteroaryl, and the C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C cycloalkoxy, 3- to 6-membered heterocycloalkoxy, C₅₋₈ cycloalkenyloxy, C₆₋₁₀ aryl or 5- to 6-membered heteroaryl is optionally substituted with one or more groups selected from the group consisting of halogen, deuterium, hydroxy, oxo, nitro and cyano.

“Cycloalkyl” or “cyclic hydrocarbon” refers to a saturated or partially unsaturated monocyclic or polycyclic hydrocarbon substituent. The cycloalkyl ring contains 3 to 20 carbon atoms, preferably 3 to 8 carbon atoms. Non-limiting examples of monocyclic cycloalkyl include cyclopropyl, cyclobutyl, cyclopentyl, cyclopentenyl, cyclohexyl, cyclohexenyl, cyclohexadienyl, and the like. Polycyclic cycloalkyl includes spiro cycloalkyl, fused cycloalkyl and bridged cycloalkyl.

The cycloalkyl ring may be fused to an aryl, heteroaryl or heterocycloalkyl ring, wherein the ring attached to the parent structure is cycloalkyl; non-limiting examples include indanyl, tetrahydronaphthyl, benzocycloheptyl, and the like. Unless otherwise specified, cycloalkyl may be optionally substituted or unsubstituted, and when it is substituted, the substituent is preferably one or more groups independently selected from the group consisting of deuterium, alkyl, alkenyl, alkynyl, alkoxy, alkylthio, alkylamino, halogen, sulfhydryl, hydroxy, nitro, cyano, cycloalkyl, heterocycloalkyl, aryl, heteroaryl, cycloalkoxy, heterocycloalkoxy, cycloalkylthio, heterocycloalkylthio, oxo, carboxyl and a carboxylate group.

“Heterocyclyl” or “heterocyclic ring” refers to a saturated or partially unsaturated monocyclic or polycyclic hydrocarbon group containing 3 to 20 ring atoms, one or more of which are heteroatoms selected from the group consisting of nitrogen, oxygen and S(O)ₘ (where m is an integer of 0 to 2), excluding a ring moiety of —O—O—, —O—S— or —S—S—, and the other ring atoms are carbon atoms. It preferably contains 3 to 12 ring atoms, 1 to 4 of which are heteroatoms; more preferably, it contains 3 to 7 ring atoms. Non-limiting examples of monocyclic heterocycloalkyl include pyrrolidinyl, imidazolidinyl, tetrahydrofuranyl, tetrahydrothienyl, dihydroimidazolyl, dihydrofuranyl, dihydropyrazolyl, dihydropyrrolyl, piperidinyl, piperazinyl, morpholinyl, thiomorpholinyl, homopiperazinyl, and the like. The polycyclic heterocycloalkyl includes spiro heterocyclyl, fused heterocyclyl and bridged heterocycloalkyl. Non-limiting examples of “heterocycloalkyl” include:

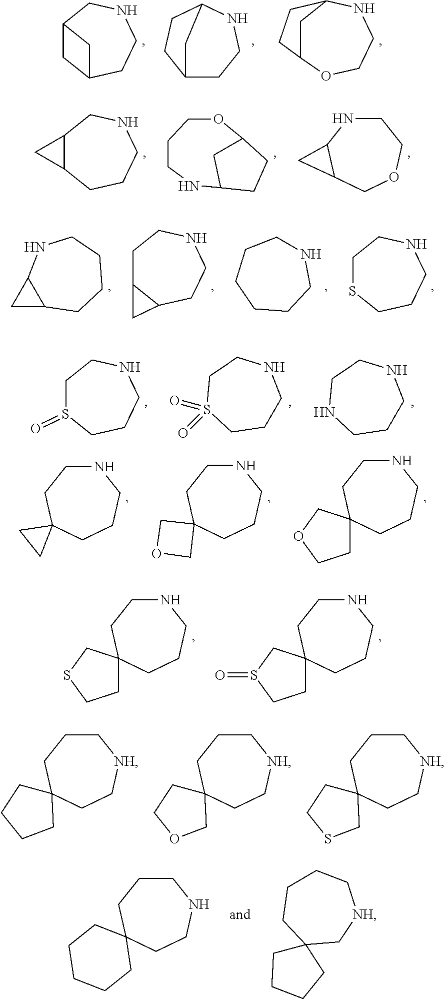

and the like.

Heterocycloalkyl may be optionally substituted or unsubstituted, and when it is substituted, the substituent is preferably one or more groups independently selected from the group consisting of, for example, halogen, deuterium, hydroxy, oxo, nitro, cyano, C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₈ cycloalkenyloxy, C₆₋₁₀ aryl or 5- to 6-membered heteroaryl, and the C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C cycloalkoxy, 3- to 6-membered heterocycloalkoxy, C₅₋₈ cycloalkenyloxy, C₆₋₁₀ aryl or 5- to 6-membered heteroaryl is optionally substituted with one or more groups selected from the group consisting of halogen, deuterium, hydroxy, oxo, nitro and cyano.

The heterocyclyl ring may be fused to an aromatic ring, a heteroaromatic ring or a cyclic hydrocarbon, wherein the ring attached to the parent structure is heterocyclyl; non-limiting examples thereof include:

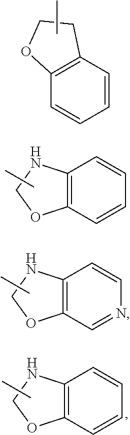

etc.

“Aryl” or “aromatic ring” refers to a 6- to 14-membered, preferably 6- to 12-membered, all-carbon monocyclic or fused polycyclic (i.e., rings sharing a pair of adjacent carbon atoms) group having a conjugated π-electron system, such as phenyl and naphthyl.

Aryl may be substituted or unsubstituted, and when it is substituted, the substituent is preferably one or more groups independently selected from the group consisting of halogen, deuterium, hydroxy, oxo, nitro, cyano, C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₈ cycloalkenyloxy, C₆₋₁₀ aryl or 5- to 6-membered heteroaryl, and the C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₈ cycloalkenyloxy, C₆₋₁₀ aryl or 5 to 6-membered heteroaryl is optionally substituted with one or more groups selected from the group consisting of halogen, deuterium, hydroxy, oxo, nitro and cyano.

The aryl ring may be fused to a heteroaromatic ring, a heterocyclic ring or a cyclic hydrocarbon, wherein the ring attached to the parent structure is the aryl ring; non-limiting examples thereof include:

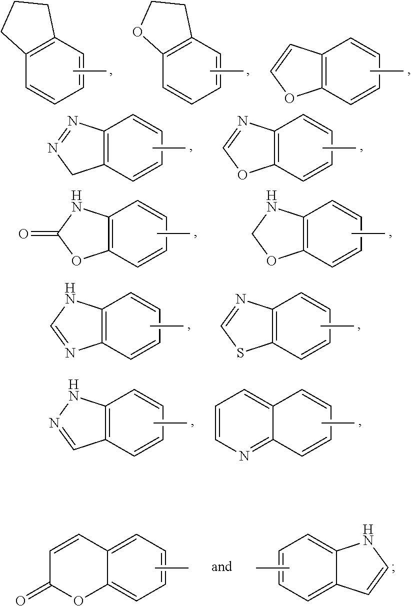

“Heteroaryl” or “heteroaromatic ring” refers to a heteroaromatic system containing 1 to 4 heteroatoms and 5 to 14 ring atoms, wherein the heteroatoms are selected from the group consisting of oxygen, sulfur and nitrogen. Heteroaryl is preferably 6- to 12-membered, more preferably 5- or 6-membered. For example, its non-limiting examples include: imidazolyl, furyl, thienyl, thiazolyl, pyrazolyl, oxazolyl, pyrrolyl, tetrazolyl, pyridinyl, pyrimidinyl, thiadiazole, pyrazine,

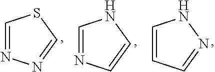

and the like.

Examples of nitrogen atom-containing heteroaryl include, but are not limited to, pyrrolyl, piperazinyl, pyrimidinyl, imidazolyl, pyridazinyl, pyrazinyl, tetrazolyl, triazolyl, pyridinyl, pyrazolyl, oxazolyl, thiazolyl, or the like.

Heteroaryl may be optionally substituted or unsubstituted, and when it is substituted, the substituent is preferably one or more groups independently selected from the group consisting of halogen, deuterium, hydroxy, oxo, nitro, cyano, C₁₋₆ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₄ cycloalkenyloxy, C₆₋₁₀ aryl or 5- to 6-membered heteroaryl, and the C₁₋₄ alkyl, C₁₋₆ alkoxy, C₂₋₆ alkenyloxy, C₂₋₆ alkynyloxy, C₃₋₆ cycloalkyl, 3- to 6-membered heterocycloalkyl, C₅₋₈ cycloalkenyl, C₃₋₆ cycloalkoxy, 3- to 6-membered heterocycloalkoxy, C₅₋₈ cycloalkenyloxy, C₆₋₁₀ aryl or 5- to 6-membered heteroaryl is optionally substituted with one or more groups selected from the group consisting of halogen, deuterium, hydroxy, oxo, nitro and cyano.

The heteroaryl ring may be fused to an aromatic ring, a heterocyclic ring or a cyclic hydrocarbon, wherein the ring attached to the parent structure is the heteroaryl ring; non-limiting examples thereof include:

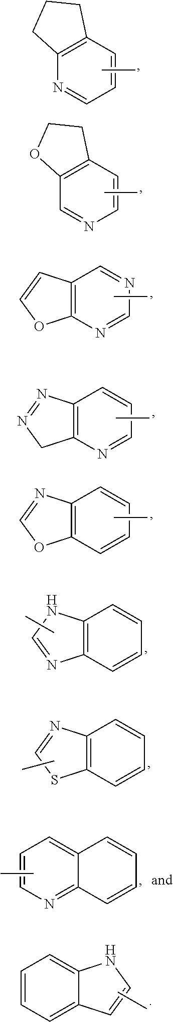

“Halogen” refers to fluorine, chlorine, bromine or iodine.

“Substituted with one or more A, B . . . ” means that it may be substituted with a single substituent or multiple substituents. In the case of substitution with multiple substituents, there may be a plurality of identical substituents, or one combination of or a plurality of combinations of different substituents.

### DETAILED DESCRIPTION

The present disclosure is further described below with reference to examples. However, these examples are not intended to limit the scope of the present disclosure.

Experimental procedures without conditions specified in the examples of the present disclosure are generally conducted according to conventional conditions, or according to conditions recommended by the manufacturer of the starting materials or commercial products. Reagents without specific origins indicated are commercially available conventional reagents.

The structures of the compounds were determined by nuclear magnetic resonance (NMR) spectroscopy and/or mass spectrometry (MS). NMR shifts (δ) were given in 10⁻⁶ (ppm). NMR analysis was performed on a Bruker AVANCE-400 nuclear magnetic resonance instrument, with deuterated dimethyl sulfoxide (DMSO-d₆), deuterated chloroform (CDCl₃) and deuterated methanol (CD₃OD) as solvents and tetramethylsilane (TMS) as an internal standard. The spatial configurations of the optical isomers (isomers) of the compounds can be further confirmed by determining single crystal parameters.

HPLC analysis was performed on Waters ACQUITY ultra high performance LC, Shimadzu LC-20A systems, Shimadzu LC-2010HT series, or Agilent 1200 LC high performance liquid chromatograph (ACQUITY UPLC BEH C18 1.7 μm 2.1×50 mm column, Ultimate XB-C18 3.0×150 mm column, or Xtimate C18 2.1×30 mm column). MS analysis was performed on a Waters SQD2 mass spectrometer in the positive/negative ion scan mode with a mass scan range of 100-1200.

Chiral HPLC analysis was performed using a Chiralpak IC-3 100×4.6 mm I.D., 3 μm; Chiralpak AD-3 150×4.6 mm I.D., 3 μm; Chiralpak AD-3 50×4.6 mm I.D., 3 μm; Chiralpak AS-3 150×4.6 mm I.D., 3 μm; Chiralpak AS-3 100×4.6 mm I.D., 3 μm; ChiralCel OD-3 150×4.6 mm I.D., 3 μm; Chiralcel OD-3 100×4.6 mm I.D., 3 μm; ChiralCel OJ-H 150×4.6 mm I.D., 5 μm; or Chiralcel OJ-3 150×4.6 mm I.D., 3 μm column.

Yantai Huanghai HSGF254 or Qingdao GF254 silica gel plates, 0.15-0.2 mm layer thickness, were adopted for thin-layer chromatography (TLC) analysis and 0.4-0.5 mm layer thickness for TLC separation and purification.

Flash column purification was performed on a Combiflash Rf150 (TELEDYNE ISCO) or Isolara one (Biotage) system.

Forward column chromatography generally used 100-200 mesh, 200-300 mesh or 300-400 mesh Yantai Huanghai silica gel as a carrier, or used a Changzhou Santai pre-fill ultrapure forward phase silica gel column (40-63 μm, 60 g, 12 g, 25 g, 40 g, 80 g or other specifications).

Reverse phase column chromatography generally used a Changzhou Santai pre-fill ultrapure C18 silica gel column (20-45 μm, 100 Å, 40 g, 80 g, 120 g, 220 g or other specifications).

High pressure column purification was performed on a Waters AutoP system in combination with a Waters XBridge BEH C18 OBD Prep Column, 130 Å, 5 μm, 19 mm×150 mm or Atlantis T3 OBD Prep Column, 100 Å, 5 μm, 19 mm×150 mm.

Preparative chiral chromatography used a DAICEL CHIRALPAK IC (250 mm×30 mm, 10 μm) or Phenomenex-Amylose-1 (250 mm×30 mm, 5 μm) column.

Known starting materials in the present disclosure may be synthesized using or according to methods known in the art, or may be purchased from Shanghai Titan Scientific, ABCR GmbH & Co. KG, Acros Organics, Aldrich Chemical Company, Accela ChemBio Inc., Darui Chemicals, and other companies.

In the examples, the reactions can all be performed in an argon atmosphere or a nitrogen atmosphere unless otherwise specified.

The argon atmosphere or nitrogen atmosphere means that the reaction flask is connected to a balloon containing about 1 L of argon or nitrogen.

The hydrogen atmosphere means that the reaction flask is connected to a balloon containing about 1 L of hydrogen.

Pressurized hydrogenation reactions were performed using a Parr 3916EKX hydrogenator and a Qinglan QL-500 hydrogenator or an HC2-SS hydrogenator.

Hydrogenation reactions generally involved 3 cycles of vacuumization and hydrogen purging.

Microwave reactions were performed on a CEM Discover-S 908860 microwave reactor.

In the examples, a solution refers to an aqueous solution unless otherwise specified.

In the examples, the reaction temperature is room temperature, i.e., 20°c to 30°c, unless otherwise specified.

### Example 1

### (R₂-(4-((1-Methylpiperidin-3-yl)amino)-5,6,7,8-tetrahydrophthalazin-1-yl)-5-(trifluoromethyl)phenol

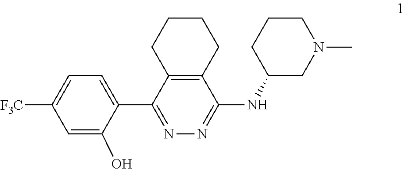 [SMILES: CN1CCC[C@@H](Nc2nnc(-c3ccc(C(F)(F)F)cc3O)c3c2CCCC3)C1]

### Step 1: Synthesis of (R)-4-chloro-N-(1-methylpiperidin-3-yl)-5,6,7,8-tetrahydrophthalazin-1-amine (compound 1c

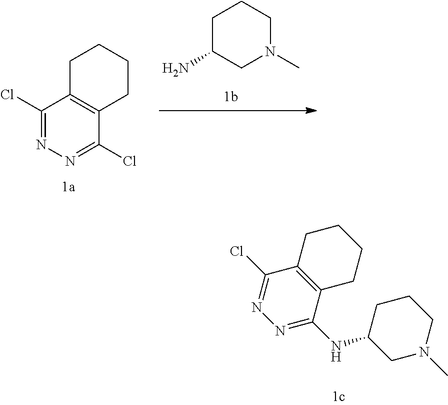

Compound 1a (100 mg, 0.492 mmol), compound 1b (56.23 mg, 0.492 mmol) and diisopropylethylamine (0.24 mL, 1.48 mmol) were mixed in NMP, and the mixture was microwaved at 180° C. until the reaction was complete. The reaction was cooled to room temperature and quenched by addition of 1 M sodium hydroxide. The mixture was extracted with dichloromethane. The organic phases were separated, combined, washed with brine, dried over anhydrous sodium sulfate and then filtered. The filtrate was concentrated in vacuo to give a crude product. The crude product was purified by flash column chromatography (eluent: 0-10% methanol in dichloromethane) to give compound 1c (30 mg, yield: 19.5%).

LCMS: t_{R}=0.575 min in 5-95AB_1min_220&254_Agilent.M ES-MS m/z 281.1 [M+H]⁺.

### Step 2: Synthesis of (R)-2-(4-((1-methylpiperidin-3-yl)amino)-5,6,7,8-tetrahydrophthalazin-1-yl)-5-(trifluoromethyl)phenol (compound 1

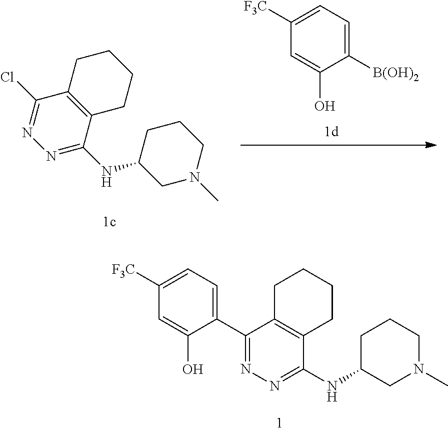 [SMILES: CN1CCC[C@@H](Nc2nnc(Cl)c3c2CCCC3)C1 ; OB(O)c1ccc(C(F)(F)F)cc1O ; CN1CCC[C@@H](Nc2nnc(-c3ccc(C(F)(F)F)cc3O)c3c2CCCC3)C1]

Compound 1c (15 mg, 0.053 mmol), compound 1d (17.60 mg, 0.085 mmol) and a 1 M sodium bicarbonate solution (0.13 mL) were mixed in dioxane (1 mL), and tetrakis(triphenylphosphine)palladium(0) (12.35 mg, 0.011 mmol) was added in a nitrogen atmosphere. The mixture was microwaved at 160° C. in a nitrogen atmosphere until the reaction was complete. The mixture was cooled to room temperature and then concentrated in vacuo to give a crude product. The crude product was purified by reversed-phase preparative HPLC [column: Boston Prime C18 150×30 mm×5 μm, 20-60% (A: water 0.05% ammonia water v/v. B: acetonitrile), flow rate: 30 mL/min] and lyophilized to give compound 1 (5.1 mg, yield: 23.7%).

LCMS: t_{R}=3.140 min in 0-95CD_7MIN.M (Waters Xbridge C18 30×2.0 mm, 3.5 μm) ES-MS m/z 407.2 [M+H]⁺.

¹H NMR: (400 MHz, DMSO-d₆) δ=7.41-7.15 (m, 3H), 3.89 (br d, J=15.3 Hz, 1H), 3.97-3.80 (m, 1H), 3.51-3.37 (m, 7H), 2.56-2.51 (m, 3H), 2.47-2.25 (m, 3H), 2.15-1.51 (m, 3H), 1.30-1.11 (m, 2H)

### Example 2

### (R)-2-(4-((1-Methylpiperidin-3-yl)amino)-6,7-dihydro-5H-cyclopenta[d]pyridazin-1-yl)-5-(trifluoromethyl)phenol

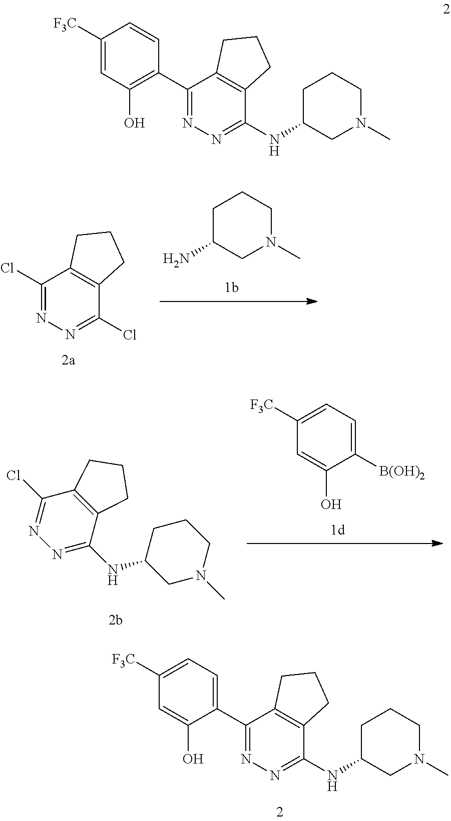

Compound 2 was prepared by using the method described in Example 1 and using 2a as the starting material.

LCMS: t_{R}=3.75 min in 0-95CD_7MIN.M (Waters Xbridge C18 30×2.0 mm, 3.5 μm) ES-MS m/z 393.2 [M+H]⁺.

¹H NMR: (400 MHz, DMSO-d₆) δ ppm 13.52 (br s, 1H), 7.75 (br d, J=8.4 Hz, 1H), 7.22 (br s, 2H), 6.35 (br d, J=7.6 Hz, 1H), 4.29-4.19 (m, 1H), 3.11 (br t, J=7.2 Hz, 2H), 2.94 (br d, J=8.8 Hz, 1H), 2.78 (br t, J=7.2 Hz, 2H), 2.69-2.60 (m, 1H), 2.18 (s, 3H), 2.12-2.01 (m, 2H), 1.96-1.81 (m, 3H), 1.75-1.66 (m, 1H), 1.62-1.49 (m, 1H), 1.44-1.30 (m, 1H).

### Example 3

### (R)-2-(4-((1-Methylpiperidin-3-yl)amino)phthalazin-1-yl)-5-(trifluoromethyl)phenol

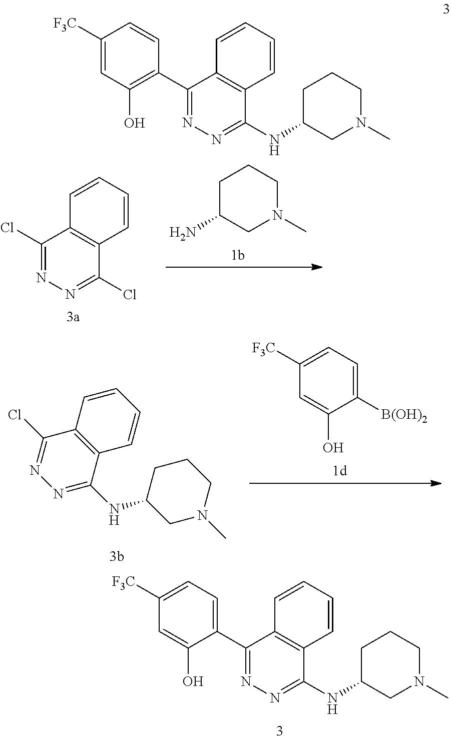

Compound 3 was prepared by using the method described in Example 1 and using 3a as the starting material.

LCMS: t_{R}=1.027 min in 10-80AB 4 min 220&254_Shimadzu.1cm (Chromolith Flash RP-C18 25-3 mm), MS (ESI) m/z=403.2 [M+H]⁺.

¹H NMR: (400 MHz, CDCl₃) δ ppm 8.88 (br s, 1H), 8.15 (d, J=8.4 Hz, 1H), 7.96 (t, J=7.6 Hz, 1H), 7.84 (t, J=7.6 Hz, 1H), 7.69 (d, J=8.0 Hz, 1H), 7.39 (s, 1H), 7.24 (d, J=8.4 Hz, 1H), 5.12 (br, 1H), 3.87-3.67 (m, 1H), 3.66-3.53 (m, 1H), 3.14-3.03 (m, 1H), 2.94-2.80 (m, 4H), 2.77 (br, 1H), 2.57-2.43 (m, 1H), 2.36 (br, 1H), 1.89 (br, 1H), 1.81-1.70 (m, 1H).

### Example 4

(R)-5-(4-Methyl-6-((1-methyl piperidin-3-yl)amino)pyridazin-3-yl)-2,3-dihydro-1H-inden-4-ol

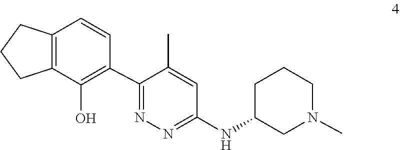 [SMILES: Cc1cc(N[C@@H]2CCCN(C)C2)nnc1-c1ccc2c(c1O)CCC2]

### Step 1: Synthesis of (compound 4c

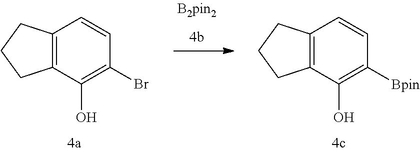

Compound 4a (250 mg, 1.173 mmol), compound 4b (1.520 mL, 5.867 mmol) and potassium acetate (345.45 mg, 3.520 mmol) were mixed in dioxane (6 mL), and Pd(dppf)Cl₂·CH₂Cl₂ (96.76 mg, 0.117 mmol) was added in a nitrogen atmosphere. The mixture was stirred at 90° C. in a nitrogen atmosphere until the reaction was complete. The mixture was cooled to room temperature and then diluted by addition of water (15 mL). The mixture was extracted with ethyl acetate (20 mL×2). The organic phases were separated, combined, washed with brine (30 mL×3), dried over anhydrous sodium sulfate and then filtered. The filtrate was concentrated in vacuo to give a crude product. The crude product was purified by flash column chromatography (eluent: 0-20% ethyl acetate in n-hexane) to give compound 4c (170 mg, yield: 55.7%).

¹H NMR (400 MHz, CDCl₃) δ ppm 7.92 (s, 1H), 7.44 (d, J=7.6 Hz, 1H), 6.82 (d, J=7.2 Hz, 1H), 2.93-2.88 (m, 4H), 2.11-2.06 (m, 2H), 1.36 (s, 12H).

### Step 2: Synthesis of (compound 4f)

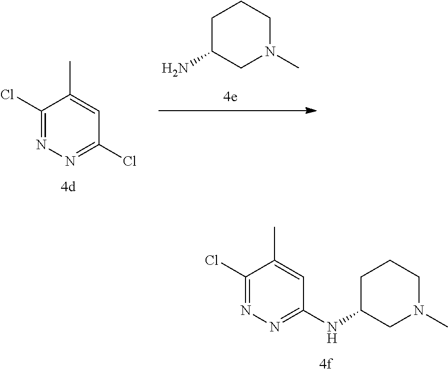

Compound 4d (200 mg, 1.227 mmol), compound 4e (168.12 mg, 1.472 mmol) and diisopropylethylamine were mixed in NMP (2 mL), and the mixture was microwaved at 120° C. until the reaction was complete. The mixture was cooled to room temperature and then diluted by addition of water (10 mL). The mixture was extracted with dichloromethane (10 mL×3). The organic phases were separated, combined, washed with brine (5 mL×3), dried over anhydrous sodium sulfate and then filtered. The filtrate was concentrated in vacuo to give a crude product. The crude product was purified by flash column chromatography (eluent: 0-10% methanol in dichloromethane) to give compound 4f (80 mg, yield: 27.1%).

### Step 3: Synthesis of (compound 4

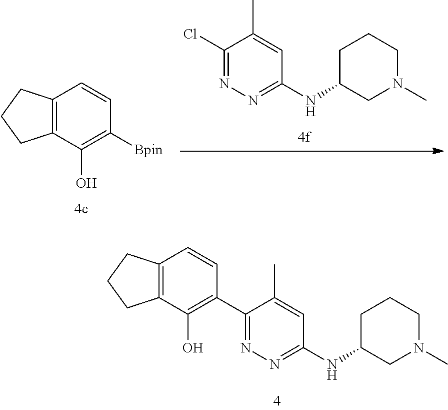

Compound 4 was prepared by using the method described in step 2 of Example 1 and using compound 4c and compound 4f as the starting materials.

LCMS: t_{R}=2.8 min in 10-80CD 7 min 220&254 Shimadzu.1cm ES-MS m/z 339.2 (M+H)⁺.

¹H NMR (400 MHz, DMSO-d₆) δ ppm 9.71 (s, 1H), 7.02 (d, J=8.0 Hz, 1H), 6.78 (d, J=7.6 Hz, 1H), 6.72 (s, 1H), 6.61 (d, J=8.0 Hz, 1H), 4.04 (s, 1H), 2.90-2.84 (m, 4H), 2.18 (s, 3H), 2.08 (s, 3H), 2.04-2.02 (m, 3H), 1.89-1.77 (m, 2H), 1.45-1.64 (m, 2H), 1.23-1.33 (m, 2H).

### Example 5

### (R)-3-(4-Methyl-6-((1-methylpiperidin-3-yl)amino)pyridazin-3-yl)naphthalen-2-ol

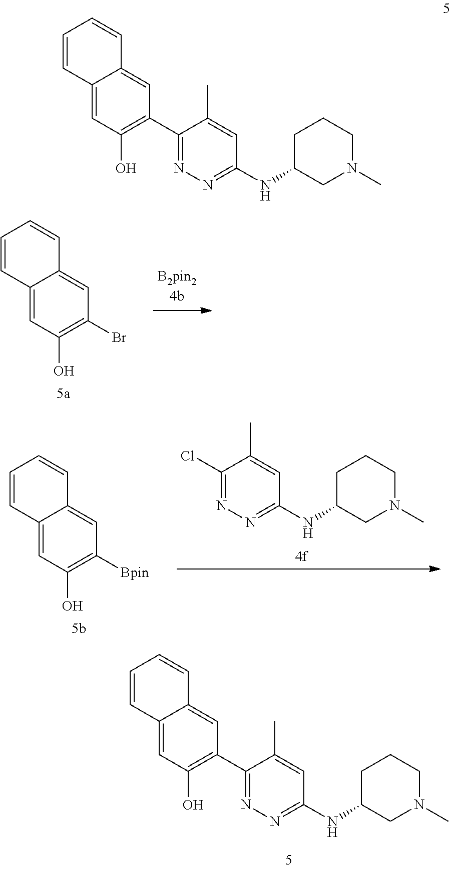

Compound 5 was prepared by using the method described in Example 4 and using compound 5a as the starting material.

¹H NMR (400 MHz, DMSO-d₆) δ ppm 8.22 (s, 1H), 7.82 (d, J=8.0 Hz, 1H), 7.72 (d, J=8 Hz, 2H), 7.42 (t, J=7.4 Hz, 1H), 7.29 (t, J=7.4 Hz, 1H), 7.24 (s, 1H), 6.71 (s, 1H), 6.65 (d, J=8.0 Hz, 1H), 4.06 (s, 1H), 2.94 (br, 1H), 2.60-2.55 (m, 1H), 2.47-2.41 (m, 1H), 2.33 (s, 1H), 2.09 (s, 1H), 1.87-1.84 (m, 1H), 1.72-1.75 (m, 1H), 1.60-1.56 (m, 1H), 1.37-1.42 (m, 1H).

### Example 6

### (R)-6-(4-Methyl-6-((1-methyl piperidin-3-yl)amino)pyridazin-3-yl)-2,3-dihydro-1H-inden-5-ol

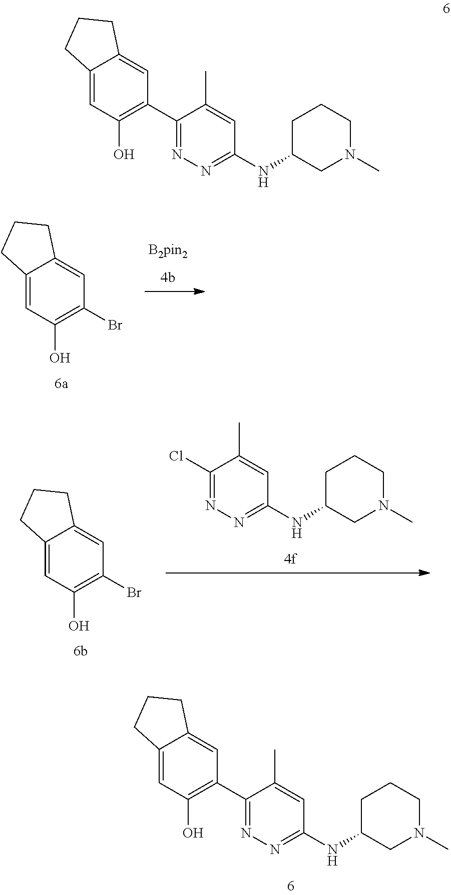 [SMILES: Cc1cc(N[C@@H]2CCCN(C)C2)nnc1-c1cc2c(cc1O)CCC2 ; Oc1cc2c(cc1Br)CCC2 ; Oc1cc2c(cc1Br)CCC2 ; Cc1cc(N[C@@H]2CCCN(C)C2)nnc1Cl ; Cc1cc(N[C@@H]2CCCN(C)C2)nnc1-c1cc2c(cc1O)CCC2]

Compound 6 was prepared by using the method described in Example 4 and using compound 6a as the starting material.

LCMS: t_{R}=2.9 min in 10-80CD 7 min 220&254 Shimadzu.1cm ES-MS m/z 339.2 (M+H)⁺.

¹H NMR (400 MHz, DMSO-d₆) δ ppm 9.47 (s, 1H), 6.97 (s, 1H), 6.76 (s, 1H), 6.65 (s, 1H), 6.50 (d, J=8.0 Hz, 1H), 4.08-3.95 (m, 1H), 2.80 (br, 5H), 2.17 (s, 3H), 2.07-1.97 (m, 6H), 1.85 (br, 3H), 1.70 (br, 1H), 1.53 (br, 1H), 1.27 (br, 1H)

### Example 7

### 4-(4-Methyl-6-((R)-1-methylpiperidin-3-yl)amino)pyridazin-3-yl)-2,3-dihydro-1H-inden-5-ol

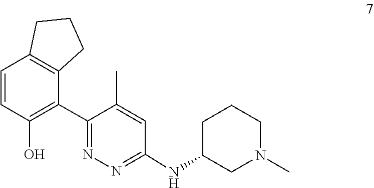 [SMILES: Cc1cc(N[C@@H]2CCCN(C)C2)nnc1-c1c(O)ccc2c1CCC2]

### Step 1: Synthesis of (compound 7b

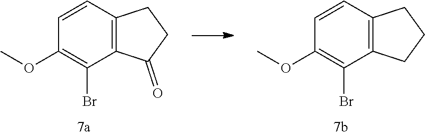

Compound 7a (100 mg, 0.415 mmol) and zinc powder (271 mg, 4.15 mmol) were mixed in acetic acid (2 mL), and the mixture was stirred at 100° C. until the reaction was complete. The mixture was cooled to room temperature and then diluted by addition of water (20 mL). The mixture was extracted with ethyl acetate (20 mL×3). The organic phases were separated, combined, washed with brine (20 mL), dried over anhydrous sodium sulfate and then filtered. The filtrate was concentrated in vacuo to give a crude product. The crude product was purified by flash column chromatography (eluent: 10-20% ethyl acetate in petroleum ether) to give compound 7b (40 mg, yield: 34.0%).

¹H NMR: (400 MHz, CDCl₃) δ ppm 7.09 (d, J=8.0 Hz, 1H), 6.70 (d, J=8.4 Hz, 1H), 3.88 (s, 3H), 2.96 (q, J=7.2 Hz, 4H), 2.15-2.06 (m, 2H). Step 2: Synthesis of (compound 7d)

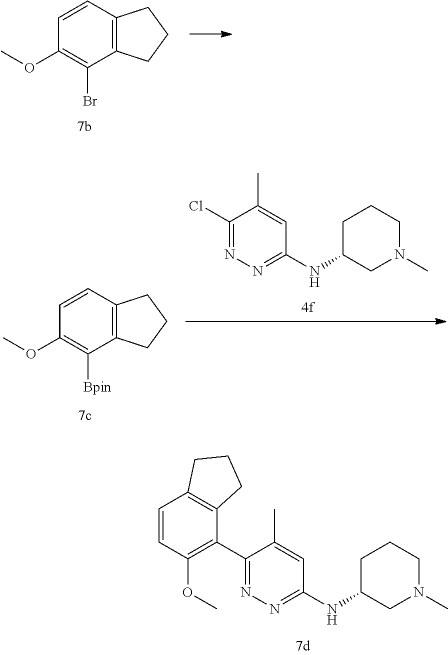

Compound 7d was prepared by using the method described in Example 4 and using compound 7b as the starting material.

LCMS: t_{R}=2.973 min in 0-30AB 7 min 220&254 Shimadzu.1cm ES-MS m/z=353.2 [M+H]⁺.

¹H NMR: ¹H NMR (400 MHz, CDCl₃) δ ppm 7.23-7.18 (m, 1H), 6.87-6.74 (m, 2H), 3.75 (s, 1H), 3.71-3.71 (m, 1H), 3.71 (s, 2H), 2.93-2.86 (m, 4H), 2.60-2.46 (m, 4H), 2.42-2.32 (m, 3H), 2.07-2.01 (m, 3H), 1.99 (s, 3H), 1.90-1.72 (m, 2H), 1.26 (br t, J=7.2 Hz, 1H).

### Step 3: Synthesis of (compound 7

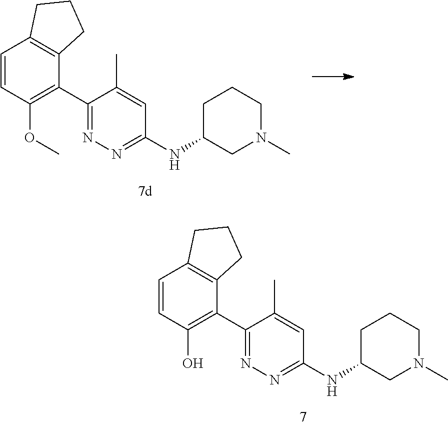 [SMILES: COc1ccc2c(c1-c1nnc(N[C@@H]3CCCN(C)C3)cc1C)CCC2 ; Cc1cc(N[C@@H]2CCCN(C)C2)nnc1-c1c(O)ccc2c1CCC2]

A solution of compound 7d (30 mg, 0.085 mmol) in dichloromethane (2 mL) was cooled to −78° C., and boron tribromide (213 mg, 0.85 mmol) was added dropwise. The mixture was warmed to room temperature and stirred until the reaction was complete. The reaction was quenched by addition of methanol (2 mL), and the solvent was removed in vacuo to give a crude product. The crude product was purified by reversed-phase preparative HPLC (Column Boston Prime C18 150×30 mm×5 μm; Condition water (0.05% ammonia hydroxide v/v)-ACN; Begin B 40; End B 70; Flow Rate (mL/min)) to give compound 7 (5.8 mg, yield: 20.1%).

LCMS: t_{R}=4.403 min in 0-60CD_7 min_220&254_Shimadzu.1cm, MS (ESI) m/z=339.3 [M+H]⁺.

¹H NMR: (400 MHz, CD₃OD) δ ppm 7.08 (d, J=8.0 Hz, 1H), 6.76 (s, 1H), 6.68 (d, J=8.0 Hz, 1H), 4.12 (dt, J=4.6, 9.2 Hz, 1H), 3.17-3.02 (m, 1H), 2.92-2.83 (m, 2H), 2.79 (br dd, J=8.0, 16.4 Hz, 2H), 2.33 (d, J=2.1 Hz, 4H), 2.20 (br d, J=6.4 Hz, 2H), 2.04 (d, J=0.8 Hz, 6H), 1.90-1.78 (m, 1H), 1.78-1.64 (m, 1H), 1.45-1.41 (m, 1H).

### Example 8

### (R)-6-(2-(Difluoromethyl)-4-(trifluoromethyl)phenyl)-5-methyl-N-(1-methylpiperidin-3-yl)pyridazin-3-amine

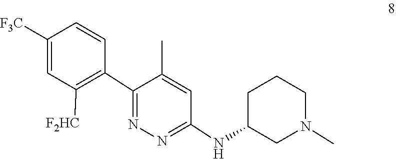 [SMILES: Cc1cc(N[C@@H]2CCCN(C)C2)nnc1-c1ccc(C(F)(F)F)cc1C(F)F]

### Step 1: Synthesis of (compound 8b

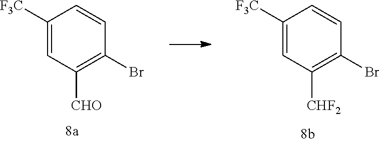 [SMILES: O=Cc1cc(C(F)(F)F)ccc1Br ; FC(F)c1cc(C(F)(F)F)ccc1Br]

Compound 8a (3.0 g, 11.86 mmol) was dissolved in dichloromethane (30 mL), and DAST (3.82 g, 23.7 mmol) was added. The mixture was stirred at room temperature until the reaction was complete. The reaction was quenched by addition of water (50 mL) and diluted by addition of dichloromethane (50 mL). The organic phase was separated, washed with saturated sodium bicarbonate solution (30 mL), dried over anhydrous sodium sulfate and then filtered. The filtrate was concentrated in vacuo to give compound 8b (3.10 g, yield: 95.1%).

¹H NMR (400 MHz, CDCl₃) Shift=7.92 (s, 1H), 7.77 (d, J=8.3 Hz, 1H), 7.59 (d, J=8.3 Hz, 1H), 6.92 (t, J=54.4 Hz, 1H)

### Step 2: Synthesis of (compound 8

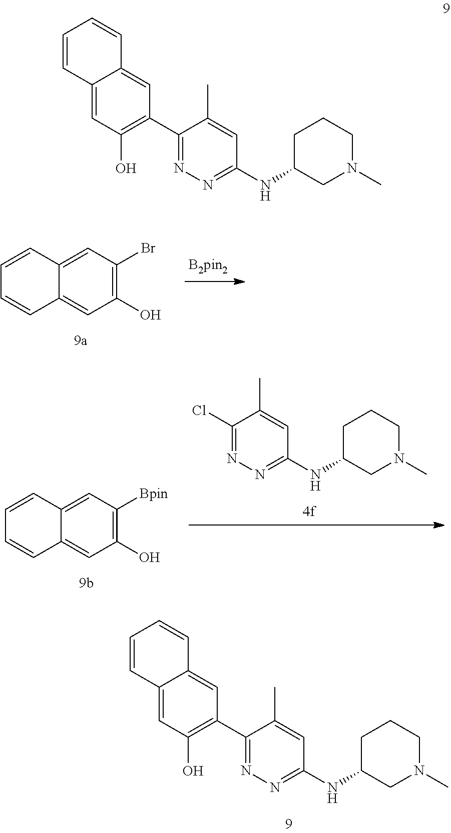

Compound 8 was prepared by using the method described in Example 4 and using compound 8b as the starting material.

LCMS: t_{R}=2.25 min in 5-95AB_7 min 220&254_Agilent.M ES-MS m/z 362.2 [M+H]⁺.

¹H NMR (400 MHz, CDCl₃) Shift=8.61 (s, 1H), 8.05 (s, 1H), 7.79 (d, J=7.9 Hz, 1H), 7.43 (d, J=8.1 Hz, 1H), 7.68 (t, J=55.4 Hz, 1H), 6.72 (s, 1H), 4.46 (br s, 2H), 3.03-2.73 (m, 2H), 2.48 (s, 4H), 2.24-1.87 (m, 5H), 1.82-1.66 (m, 2H)

### Example 9

### (R)-3-(4-Methyl-6-((1-methylpiperidin-3-yl)amino)pyridazin-3-yl)naphthalen-2-ol

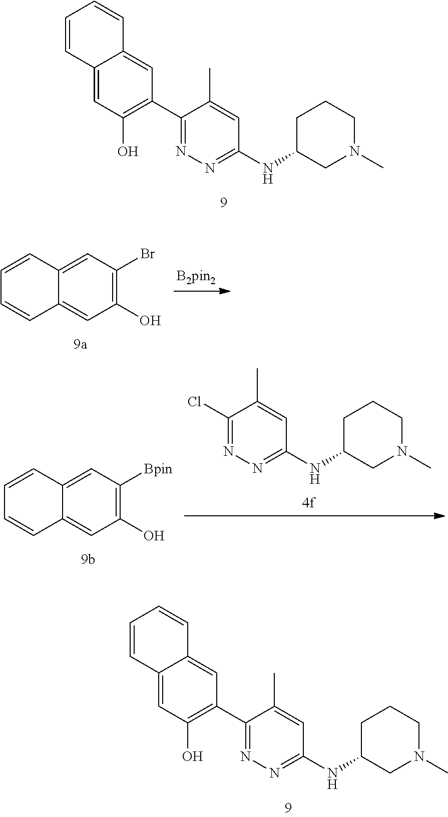

Compound 9 was prepared by using the method described in Example 4 and using compound 9a as the starting material.

LCMS: t_{R}=2.76 min in 10-80CD 7 min 220&254 Shimadzu.1cm ES-MS m/z 349.2 [M+H]⁺.

### Example 10

### (R)-1-(6-((1-Methylpiperidin-3-yl)amino)pyridazin-3-yl)naphthalen-2-ol

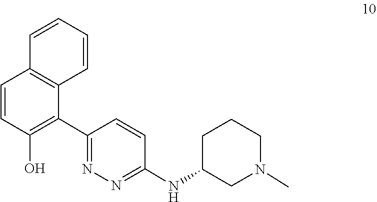 [SMILES: CN1CCC[C@@H](Nc2ccc(-c3c(O)ccc4ccccc34)nn2)C1]

### Step 1: Synthesis of (compound 10b

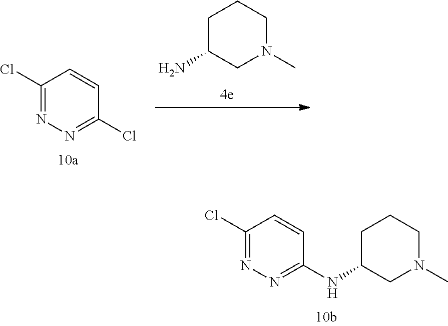

Compound 10b was prepared by using the method described in step 2 of Example 4 and using compound 10a as the starting material.

¹H NMR: (400 MHz, CDCl₃) δ ppm 7.35 (d, J=9.2 Hz, 1H), 6.93 (d, J=9.2 Hz, 1H), 4.00-3.94 (m, 1H), 2.79-2.75 (m, 1H), 2.49-2.47 (m, 1H), 2.16 (s, 3H), 2.06-2.03 (m, 1H), 1.93-1.90 (m, 1H), 1.77-1.75 (m, 1H), 1.69-1.67 (m, 1H), 1.54-1.49 (m, 1H), 1.28-1.21 (m, 1H).

### Step 2: Synthesis of (compound 10

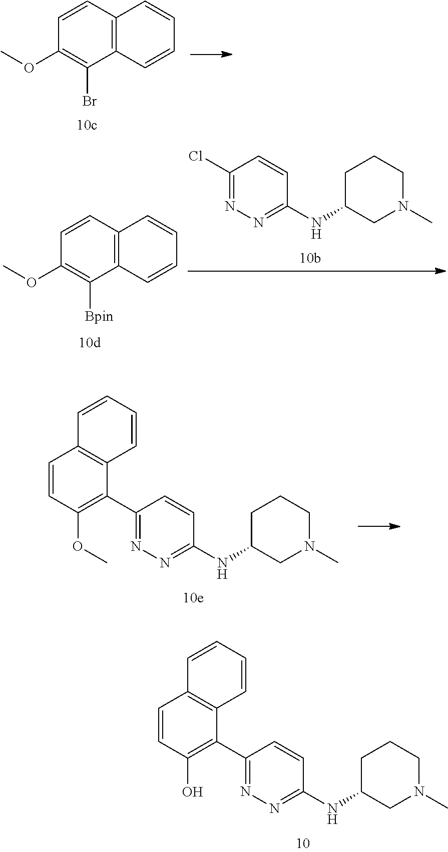

Compound 10 was prepared by using the method described in Example 7 and using compound 10c as the starting material.

LCMS: ES-LCMS m/z 335.2 [M+H]⁺.

¹H NMR: (400 MHz, CDCl₃) δ ppm 7.90 (d, J=8.4 Hz, 1H), 7.85-7.74 (m, 3H), 7.43 (t, J=7.2 Hz, 1H), 7.37-7.33 (m, 1H), 7.30 (d, J=9.2 Hz, 1H), 6.89 (br d, J=9.2 Hz, 1H), 4.34 (br s, 1H), 2.83-2.51 (m, 3H), 2.36 (br s, 3H), 1.89 (br s, 5H), 1.26 (br s, 1H).

### Example 11

### rac-(R₂-(4-Methyl-6-((1-methylpiperidin-3-yl)amino)pyridazin-3-yl)-5-( (trifluoromethyl)thio)phenol

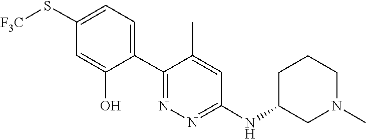 [SMILES: Cc1cc(N[C@@H]2CCCN(C)C2)nnc1-c1ccc(SC(F)(F)F)cc1O]

### Step 1: Synthesis of (compound 11b

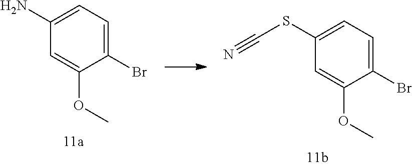

Compound 11a (2 g, 10.0 mmol) was mixed with 50% sulfuric acid (40 mL), the mixture was cooled to −5° C., a solution of sodium nitrite (759 mg, 11 mmol) (20 mL) was added dropwise, the temperature was maintained, and then cuprous thiocyanate (20 mmol) and potassium thiocyanate (20 mmol) were added. The final mixture was stirred at 0° C. until the reaction was complete. The mixture was filtered, and the filter cake was collected and dissolved in ethyl acetate (50 mL). The solution was washed with brine (30 mL), dried over anhydrous sodium sulfate and then filtered. The filtrate was concentrated in vacuo to give a crude product. The crude product was purified by flash column chromatography (eluent: 2-10% ethyl acetate in petroleum ether) to give compound 11b (0.5 g, yield: 20.7%).

¹H NMR: (400 MHz, CDCl₃) δ ppm 7.59 (d, J=8.0 Hz, 1H), 7.04-6.98 (m, 2H), 3.95 (s, 3H).

### Step 2: Synthesis of (compound 11c

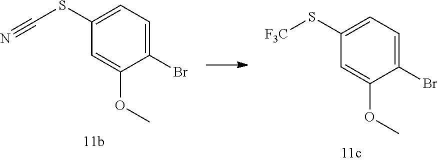

Potassium trifluoroacetate (374 mg, 2.46 mmol), ferrous chloride (78 mg, 0.61 mmol) and compound 11b (500 mg, 2.05 mmol) were mixed in DMF (5 mL), and the mixture was stirred at 140° C. in a nitrogen atmosphere until the reaction was complete. The mixture was diluted by addition of water (20 mL) and extracted with ethyl acetate (20 mL×3). The organic phases were separated, combined, washed with brine (20 mL×3), dried over anhydrous sodium sulfate and then filtered. The filtrate was concentrated in vacuo to give a crude product. The crude product was purified by flash column chromatography (eluent: 0-3% ethyl acetate in petroleum ether) to give compound 11c (40 mg, yield: 34.0%).

¹H NMR: (400 MHz, DMSO-d₆) δ ppm 7.76 (d, J=8.4 Hz, 1H), 7.39 (d, J=1.6 Hz, 1H), 7.24 (dd, J=1.6, 8.0 Hz, 1H), 3.91 (s, 3H).

### Step 3: Synthesis of (compound 11

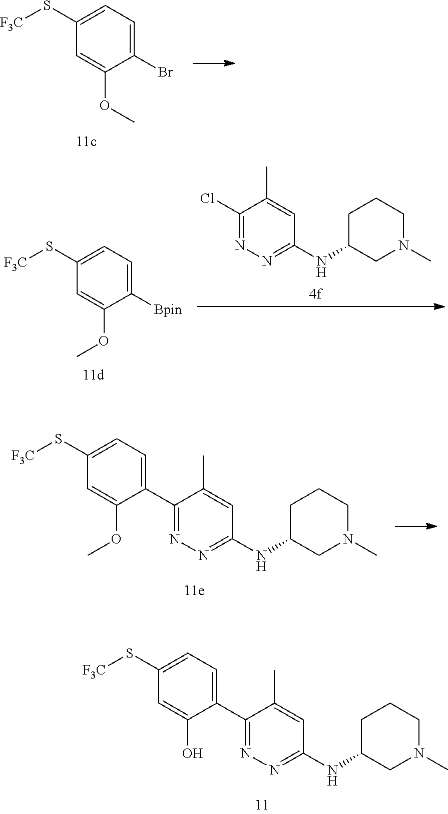

Compound 11 was prepared by using the method described in Example 7 and using compound 11c as the starting material.

LCMS: t_{R}=1.161 min in 10-80AB_7 min_220&254_Shimadzu.1cm; MS (ESI) m/z=399.2 [M+H]⁺.

¹H NMR: (400 MHz, CDCl₃) δ ppm 8.58 (s, 1H), 7.48 (d, J=8.0 Hz, 1H), 7.42-7.37 (m, J=1.5 Hz, 1H), 7.17 (d, J=8.4 Hz, 1H), 6.85 (s, 1H), 4.56 (br s, 1H), 3.09 (br s, 2H), 2.79 (br s, 2H), 2.55 (br s, 3H), 2.46 (s, 4H), 1.82-1.63 (m, J=14.9 Hz, 2H), 1.26 (s, 1H).

### Example 12

### (R)-3-Hydroxy-4-(4-methyl-6-((1-methylpiperidin-3-yl)amino)pyridazin-3-yl)benzoic acid

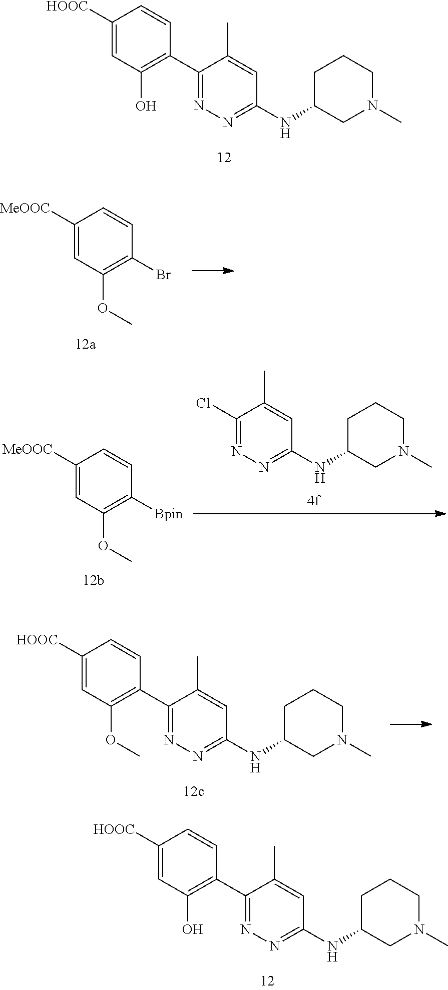

Compound 12 was prepared by using the method described in Example 7 and using compound 12a as the starting material.

LCMS: ES-LCMS m/z 343.1 [M+H]⁺.

¹H NMR: (400 MHz, DMSO-d₆) δ ppm 13.97 (br s, 1H), 8.08 (s, 1H), 7.87 (d, J=8.4 Hz, 1H), 7.44-7.36 (m, 2H), 6.21 (br d, J=7.6 Hz, 1H), 4.24 (br d, J=8.4 Hz, 1H), 2.92 (br d, J=7.6 Hz, 2H), 2.61 (br s, 2H), 2.23 (s, 3H), 2.19 (s, 3H), 1.97-1.90 (m, 2H), 1.86 (br s, 1H), 1.73-1.67 (m, 1H), 1.62-1.53 (m, 1H), 1.48-1.39 (m, 1H).

### Example 13

### (R)-4-(4-Methyl-6-((1-methylpiperidin-3-yl)amino)pyridazin-3-yl)pyridin-3-ol

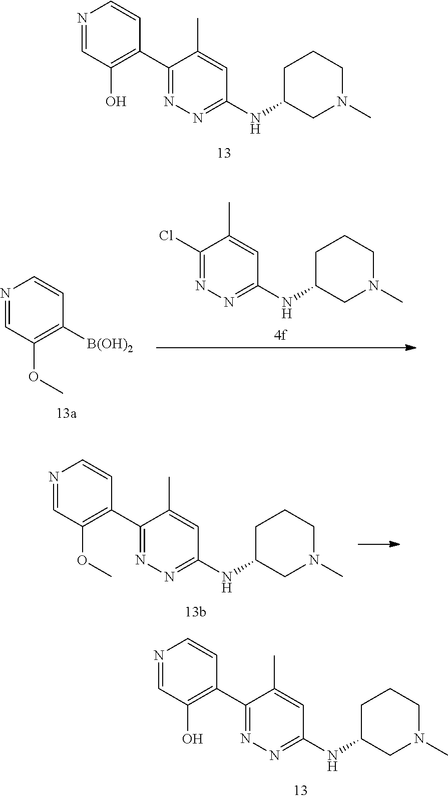 [SMILES: Cc1cc(N[C@@H]2CCCN(C)C2)nnc1-c1ccncc1O ; COc1cnccc1B(O)O ; Cc1cc(N[C@@H]2CCCN(C)C2)nnc1Cl ; COc1cnccc1-c1nnc(N[C@@H]2CCCN(C)C2)cc1C ; Cc1cc(N[C@@H]2CCCN(C)C2)nnc1-c1ccncc1O]

Compound 13 was prepared by using the method described in Example 7 and using compound 13a as the starting material.

¹H NMR: (400 MHz, CDCl₃) δ=8.48 (s, 1H), 8.16 (d, J=5.2 Hz, 1H), 7.35 (d, J=5.2 Hz, 1H), 6.67 (s, 1H), 5.57 (br s, 1H), 4.20 (br s, 1H), 2.55 (br s, 2H), 2.49 (s, 3H), 2.30 (s, 3H), 2.24-2.18 (m, 1H), 1.91-1.52 (m, 6H).

### Example 14

### 5-(6-((1s,3s)-3-Hydroxy-3-methylcyclobutyl)amino)-4-methylpyridazin-3-yl)-2,3-dihydro-1H-inden-4-ol

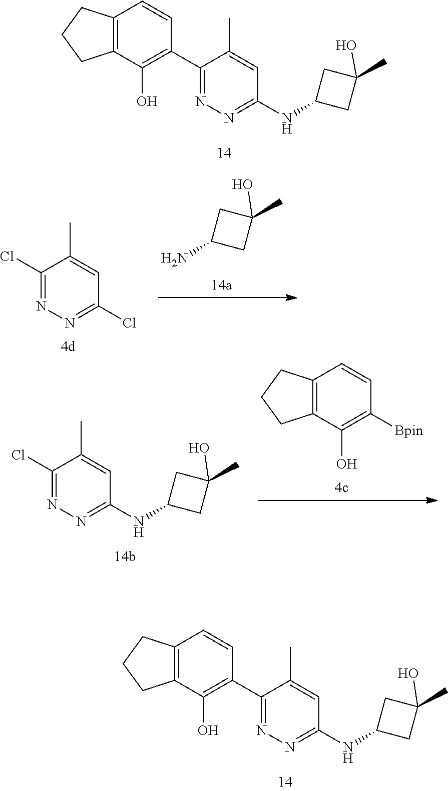

### Step 1: Synthesis of (compound 14b

Compound 14b was prepared by using the method described in step 2 of Example 4 and using compound 14a as the starting material.

LCMS: t_{R}=0.4 min in 5-95AB_1min_220&254_Agilent.M ES-MS m/z 228.1 [M+H]⁺.

¹H NMR: (400 MHz, CDCl₃) δ ppm 7.03 (s, 1H), 4.42 (br s, 1H), 4.28 (br s, 1H), 3.87 (br s, 1H), 2.73 (br s, 2H), 2.44 (s, 3H), 2.20-2.16 (m, 2H), 1.46 (s, 3H).

### Step 2: Synthesis of (compound 14

Compound 14 was prepared by using the method described in step 2 of Example 1 and using compound 14b as the starting material.

LCMS: t_{R}=2.761 min in 0-95CD_7MIN.M (Waters Xbridge C18 30×2.0 mm, 3.5 μm) ES-MS m/z 326.2 [M+H]⁺.

¹H NMR: (400 MHz, DMSO-d₆) δ ppm 9.70 (br s, 1H), 7.01 (d, J=7.6 Hz, 1H), 6.95 (d, J=6.8 Hz, 1H), 6.77 (d, J=7.6 Hz, 1H), 6.64 (s, 1H), 4.99 (s, 1H), 3.97-3.85 (m, 1H), 2.89-2.82 (m, 4H), 2.45-2.37 (m, 2H), 2.09 (s, 3H), 2.07-1.99 (m, 2H), 1.98-1.90 (m, 2H), 1.28 (s, 3H).

### Example 15

### 2-(4-((1s,3s)-3-Hydroxy-3-methylcyclobutyl)amino)phthalazin-1-yl)-5-(trifluoromethyl)phenol

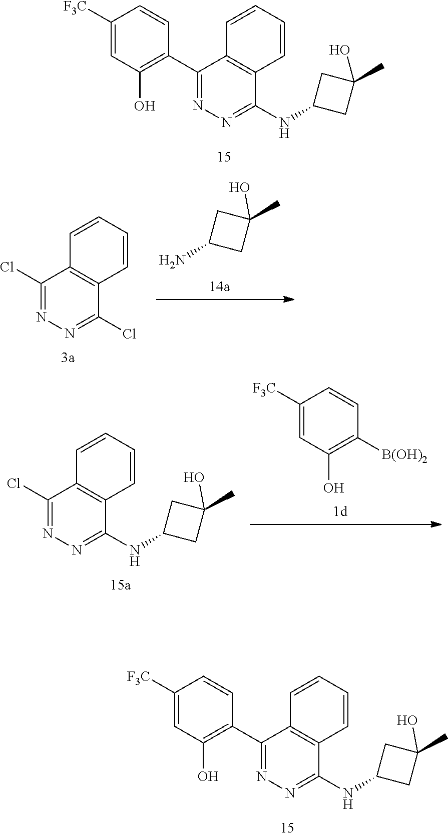

Compound 15 was prepared by using the method described in Example 14 and using 3a as the starting material.

LCMS: t_{R}=2.415 min in 0-95CD_7MIN.M (Waters Xbridge C18 30×2.0 mm, 3.5 μm) ES-MS m/z 390.1 [M+H]⁺.

¹H NMR: (400 MHz, DMSO-d₆) δ ppm 10.34 (br s, 1H), 8.42 (d, J=8.4 Hz, 1H), 7.89-7.82 (m, 1H), 7.77 (t, J=7.2 Hz, 1H), 7.62 (br d, J=5.6 Hz, 1H), 7.50 (d, J=7.8 Hz, 1H), 7.43 (d, J=8.0 Hz, 1H), 7.32-7.24 (m, 2H), 5.00 (s, 1H), 4.31-4.14 (m, 1H), 2.46 (br d, J=2.8 Hz, 2H), 2.22-2.13 (m, 2H), 1.33 (s, 3H).

### Example 16

### 2-(4-((1 s,3s)-3-Hydroxy-3-methylcyclobutyl)amino)-5,7-dihydrofuro[3,4-d]pyridazin-1-yl)-5-(trifluoromethyl)phenol

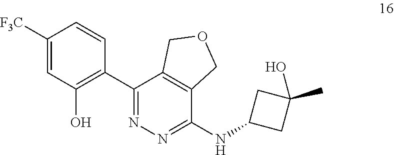 [SMILES: C[C@]1(O)C[C@@H](Nc2nnc(-c3ccc(C(F)(F)F)cc3O)c3c2COC3)C1]

### Step 1: Synthesis of (compound 16b

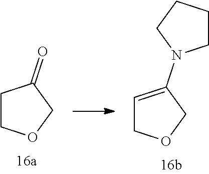 [SMILES: O=C1CCOC1 ; C1=C(N2CCCC2)COC1]

Compound 16a (1 g, 11.6 mmol) and potassium carbonate (0.16 g, 1.16 mmol) were mixed in tetrahydropyrrole (0.95 mL, 11.6 mmol), and the mixture was stirred at 0° C. until the reaction was complete. The mixture was filtered, and the filtrate was concentrated in vacuo to give compound 16b (1.5 g, yield: 92.8%).

LCMS: ES-LCMS m/z 140.1 [M+H]⁺.

### Step 2: Synthesis of (compound 16c

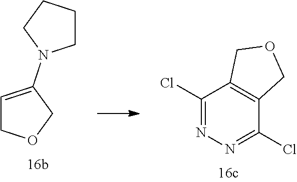 [SMILES: C1=C(N2CCCC2)COC1 ; Clc1nnc(Cl)c2c1COC2]

Compound 16b (300 mg, 2.16 mmol) and 3,6-dichlorotetrazine (325 g, 2.16 mmol) were mixed in dichloromethane (5 mL), and the mixture was stirred at 0° C. until the reaction was complete. The mixture was diluted by addition of water (5 mL) and extracted with dichloromethane (5 mL×3). The organic phases were separated, combined, washed with brine (5 mL), dried over anhydrous sodium sulfate and then filtered. The filtrate was concentrated in vacuo to give a crude product. The crude product was purified by flash column chromatography (eluent: 0-20% ethyl acetate in petroleum ether) to give compound 16c (70 mg, yield: 15.3%).

LCMS: t_{R}=0.568 min in 5-95AB_1min 220&254_Agilent.M ES-MS m/z 191.0 (M+H)+

¹H NMR: (400 MHz, CDCl₃) δ ppm 5.24 (s, 4H)

### Step 3: Synthesis of (compound 16)

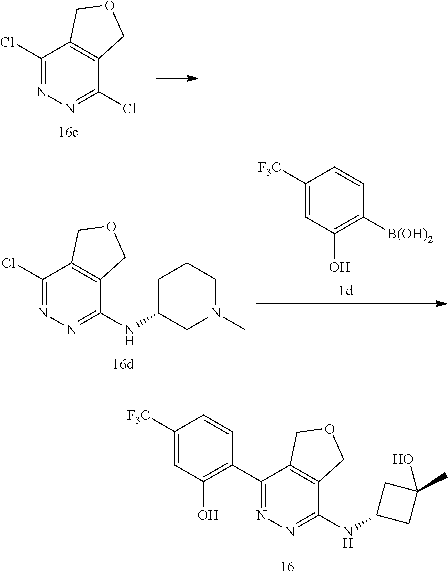 [SMILES: Clc1nnc(Cl)c2c1COC2 ; CN1CCC[C@@H](Nc2nnc(Cl)c3c2COC3)C1 ; OB(O)c1ccc(C(F)(F)F)cc1O ; C[C@]1(O)C[C@@H](Nc2nnc(-c3ccc(C(F)(F)F)cc3O)c3c2COC3)C1]

Compound 16 was prepared by using the method described in Example 1 and using 16c as the starting material.

¹H NMR (400 MHz, DMSO-d₆) δ=8.14 (s, 1H), 7.47 (br d, J=8.0 Hz, 1H), 7.26-7.22 (m, 2H), 6.88 (br d, J=7.0 Hz, 1H), 5.26 (br s, 2H), 4.97 (br s, 2H), 4.30 (br s, 1H), 2.33 (br s, 3H), 2.15-1.88 (m, 4H), 1.77 (br s, 1H), 1.61 (br d, J=12.0 Hz, 1H), 1.38 (br d, J=12.3 Hz, 1H), 1.23 (br s, 2H).

### Example 17

### 4-(4-Methyl-6-((R)-1-methylpiperidin-3-yl)amino)pyridazin-3-yl)-2,3-dihydro-1H-inden-5-ol

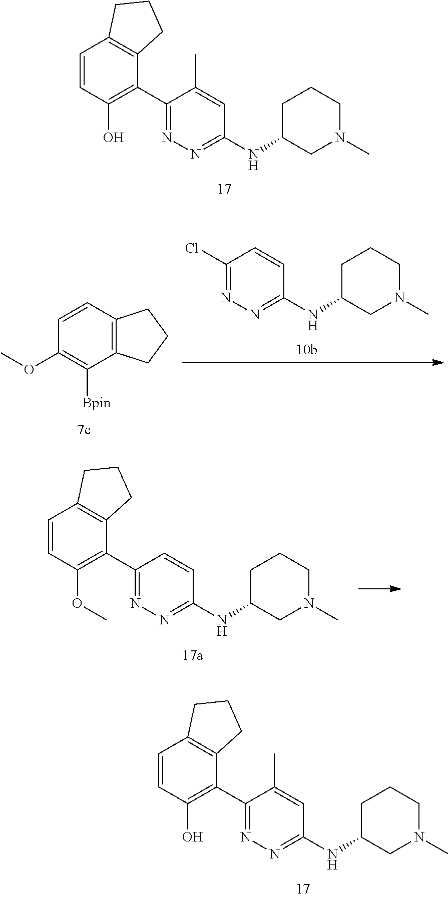

Compound 17 was prepared by using the method described in Example 7 and using 7c as the starting material.

LCMS: ES-LCMS m/z 339.2 [M+H]⁺.

### Example 18

### (R)-5-Cyclopropyl-2-(4-methyl-6-((1-methylpiperidin-3-yl)amino)pyridazin-3-yl)phenol

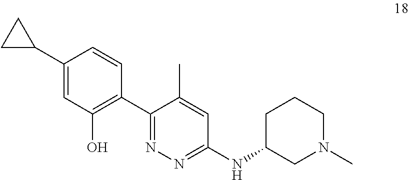 [SMILES: Cc1cc(N[C@@H]2CCCN(C)C2)nnc1-c1ccc(C2CC2)cc1O]

### Step 1: Synthesis of (compound 18b

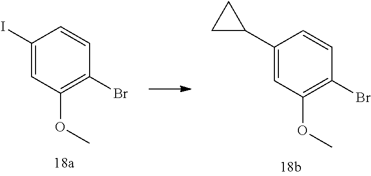

Compound 18a (500 mg, 1.6 mmol), cyclopropylboronic acid (505 mg, 5.87 mmol) and potassium carbonate (883 mg, 6.39 mmol) were mixed in dioxane (10 mL) and water (5 mL), and Pd(dppf)Cl₂·CH₂Cl₂ (65.9 mg, 0.080 mmol) was added in a nitrogen atmosphere. The mixture was stirred at 120° C. in a nitrogen atmosphere until the reaction was complete. The mixture was cooled to room temperature and then diluted by addition of ethyl acetate (20 mL). The organic phase was washed with brine (30 mL), dried over anhydrous sodium sulfate and then filtered. The filtrate was concentrated in vacuo to give crude compound 18b. The crude product was directly used in the next step.

### Step 2: Synthesis of (compound 18)

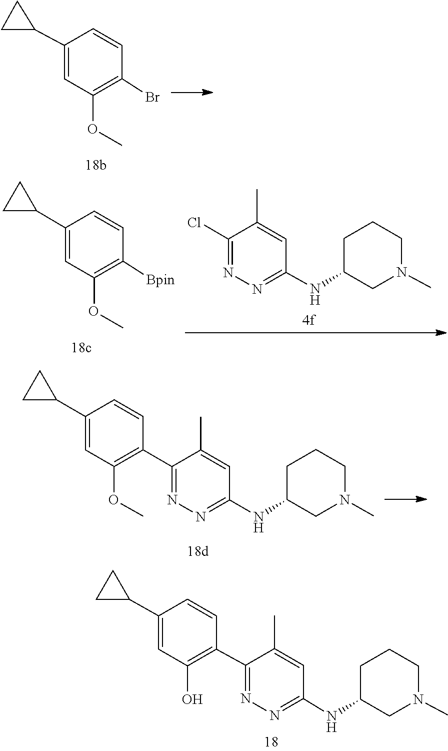

Compound 18 was prepared by using the method described in Example 7 and using 18b as the starting material.

LCMS: ES-LCMS m/z 339.3 [M+H]⁺.

¹H NMR (400 MHz, CDCl₃) δ=7.20 (d, J=8.0 Hz, 1H), 6.73 (d, J=8.0 Hz, 1H), 6.69 (s, 1H), 6.63 (br s, 1H), 4.32 (br s, 1H), 2.95-2.45 (m, 4H), 2.41 (s, 3H), 2.36-2.19 (m, 1H), 2.05 (s, 3H), 1.98-1.92 (m, 1H), 1.88-1.54 (m, 4H), 1.05-0.95 (m, 2H), 0.81-0.71 (m, 2H).

### Example 19

### rac-(R)-2-(4-(1-(2-Hydroxyethyl)piperidin-3-yl)amino)-5,6,7,8-tetrahydrophthalazin-1-yl)-5-(trifluoromethyl)phenol

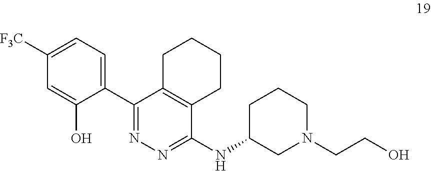 [SMILES: OCCN1CCC[C@@H](Nc2nnc(-c3ccc(C(F)(F)F)cc3O)c3c2CCCC3)C1]

### Step 1: Synthesis of (compound 19c

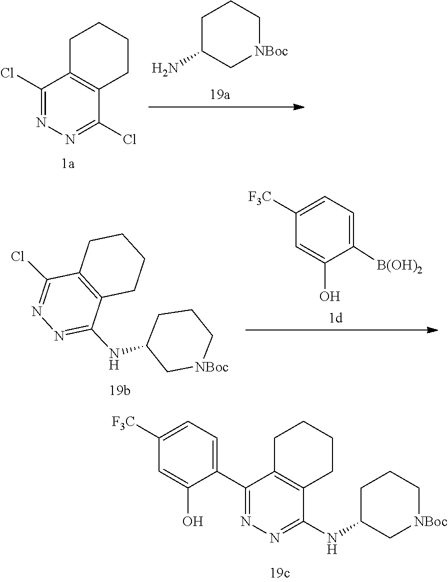

Compound 19c was prepared by using the method described in Example 1 and using 19a as the starting material.

LCMS: ES-LCMS m/z 493.3 [M+H]⁺.

### Step 2: Synthesis of (compound 19d

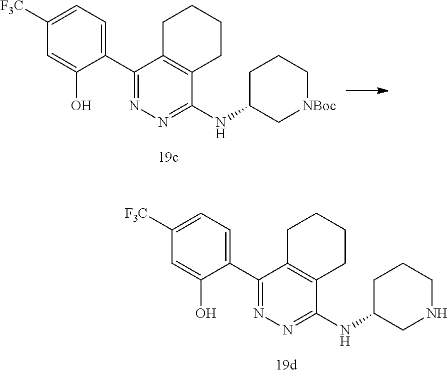 [SMILES: CC(C)(C)OC(=O)N1CCC[C@@H](Nc2nnc(-c3ccc(C(F)(F)F)cc3O)c3c2CCCC3)C1 ; Oc1cc(C(F)(F)F)ccc1-c1nnc(N[C@@H]2CCCNC2)c2c1CCCC2]

Compound 19c (180 mg, 0.365 mmol) was dissolved in a solution of hydrochloric acid in dioxane (2 mL), and the mixture was stirred at room temperature until the reaction was complete. The mixture was concentrated in vacuo to give the hydrochloride of compound 19d (220 mg, yield: 92.0%).

LCMS: ES-LCMS m/z 393.3 [M+H]⁺.

### Step 3: Synthesis of (compound 19

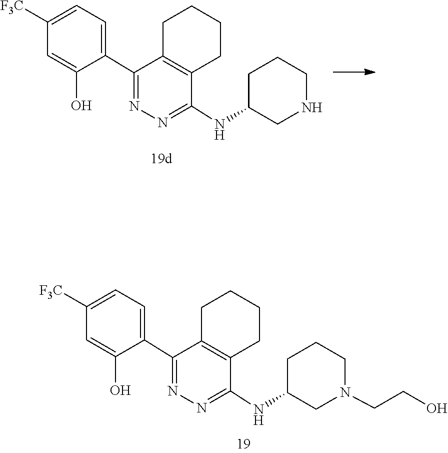 [SMILES: Oc1cc(C(F)(F)F)ccc1-c1nnc(N[C@@H]2CCCNC2)c2c1CCCC2 ; OCCN1CCC[C@@H](Nc2nnc(-c3ccc(C(F)(F)F)cc3O)c3c2CCCC3)C1]

Compound 19d (20 mg, 0.05 mmol) and diisopropylethylamine (0.017 mL, 0.10 mmol) were dissolved in acetonitrile (2 mL), and bromoethanol (6.37 mg, 0.05 mmol) was added. The mixture was stirred at 70° C. until the reaction was complete. The mixture was concentrated in vacuo to give a crude product. The crude product was purified by reversed-phase preparative HPLC to give compound 19 (2.9 mg, yield: 12.9%).

LCMS: ES-LCMS m/z 437.2 [M+H]⁺.

¹H NMR (400 MHz, CD₃OD) δ=7.37 (d, J=7.8 Hz, 1H), 7.24 (d, J=8.0 Hz, 1H), 7.18 (s, 1H), 4.71-4.60 (m, 2H), 3.80-3.55 (m, 4H), 3.06 (br d, J=7.3 Hz, 2H), 2.55-2.44 (m, 4H), 2.21-2.07 (m, 2H), 2.03-1.84 (m, 4H), 1.83-1.70 (m, 3H).

### Example 20

### rac-3-Fluoro-2-(4-((R)-1-methylpiperidin-3-yl)amino)-6,7-dihydro-5H-cyclopenta[d]pyridazin-1-yl)phenol

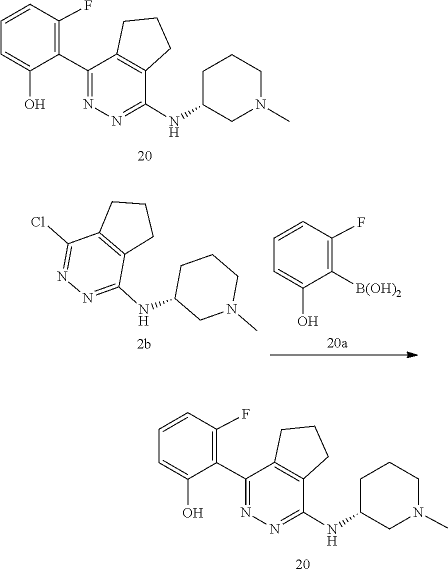

Compound 20 was prepared by using the method described in Example 2 and using 2b as the starting material.

LCMS: t_{R}=0.719 min in 5-95AB_1min 220&254_Agilent.M ES-MS m/z 343.1 [M+H]⁺.

¹H NMR (400 MHz, CD₃OD) Shift=7.26 (dt, J=6.8, 8.3 Hz, 1H), 6.78-6.66 (m, 2H), 4.48-4.38 (m, 1H), 3.09 (br s, 1H), 2.88 (t, J=7.5 Hz, 2H), 2.80 (t, J=7.7 Hz, 2H), 2.70 (br s, 1H), 2.36 (s, 3H), 2.31 (br d, J=13.1 Hz, 2H), 2.15 (quin, J=7.7 Hz, 2H), 2.00 (br s, 1H), 1.92-1.81 (m, 1H), 1.79-1.68 (m, 1H), 1.57 (br s, 1H)

### Example 21

### 5-(6-((1s,3s)-3-Hydroxy-3-methylcyclobutyl)amino)-4,5-dimethylpyridazin-3-yl)-2,3-dihydro-1H-inden-4-ol

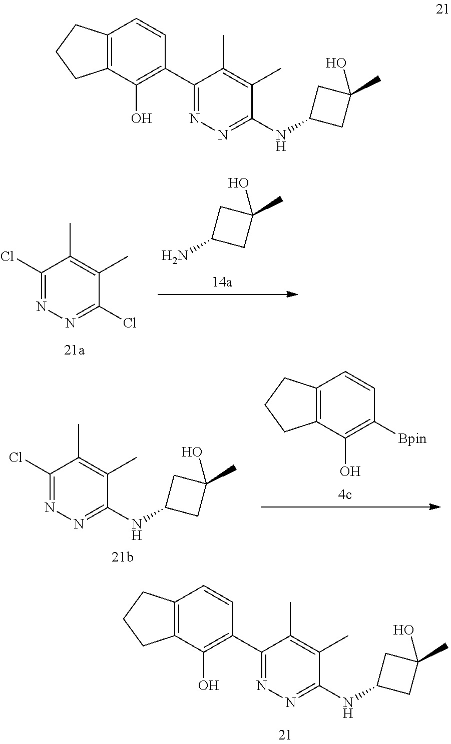

Compound 21 was prepared by using the method described in Example 14 and using 21a as the starting material.

LCMS: t_{R}=0.774 min in 5-95AB_1min 220&254_Agilent.M ES-MS m/z 340.2 [M+H]⁺.

¹H NMR (400 MHz, CDCl₃) Shift=7.05 (d, J=7.8 Hz, 1H), 6.81 (d, J=7.8 Hz, 1H), 4.62 (br s, 1H), 4.41-4.24 (m, 1H), 2.97 (td, J=7.4, 19.0 Hz, 4H), 2.75 (ddd, J=2.8, 7.4, 9.9 Hz, 2H), 2.35 (s, 3H), 2.18-2.06 (m, 8H), 1.47 (s, 3H)

### Example 22

### 2-(5-((1 s,3s)-3-Hydroxy-3-methylcyclobutyl)amino)pyrido[2,3-d]pyridazin-S-yl)-5-(trifluoromethyl)phenol (compound 22

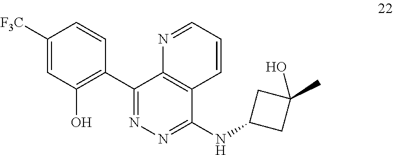 [SMILES: C[C@]1(O)C[C@@H](Nc2nnc(-c3ccc(C(F)(F)F)cc3O)c3ncccc23)C1]

### 2-(8-((1 s,3s)-3-Hydroxy-3-methylcyclobutyl)amino)pyrido[2,3-d]pyridazin-5-yl)-5-(trifluoromethyl)phenol (compound 23

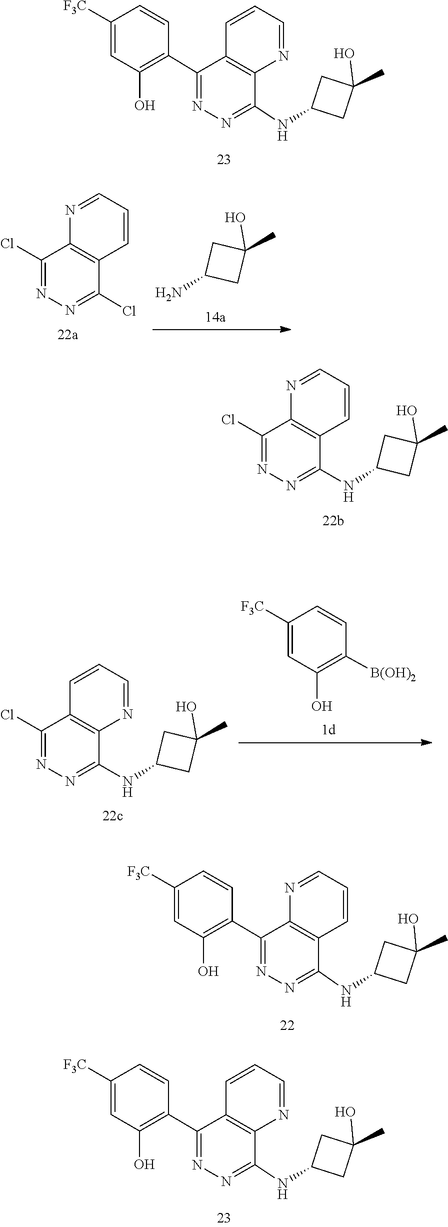

Compounds 22 and 23 were prepared by using the method described in Example 15 and using 22a as the starting material.

### Compound 22

¹H NMR: (400 MHz,CD₃OD) δ ppm 9.08 (m, 1H), 8.00 (d, J=8.0 Hz, 1H), 7.80 (dd, J=8.0, 4.4 Hz, 1H), 7.58 (d, J=8.0 Hz, 1H), 7.31 (d, J=8.0 Hz, 1H), 7.26 (s, 1H), 4.39-4.31 (m, 1H), 2.72-2.67 (m, 2H), 2.27-2.21 (m, 2H), 1.45 (s, 3H).

### Compound 23

¹H NMR: (400 MHz,CD₃OD) δ ppm 9.15 (br s, 1H), 8.80 (dd, J=8.4, 2.4 Hz, 1H), 8.49 (d, J=8.0 Hz, 1H), 7.89 (d, J=8.4 Hz, 1H), 7.22 (d, J=6.8 Hz, 1H), 4.34-4.26 (m, 1H), 2.71-2.66 (m, 2H), 2.27-2.22 (m, 2H), 1.46 (s, 3H).

### Example 23

### 2-(4-((1s,3s)-3-Hydroxy-3-methylcyclobutyl)amino)pyrido[3,4-d]pyridazin-1-yl)-5-(trifluoromethyl)phenol (compound 24

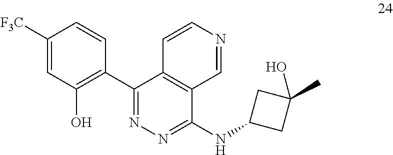 [SMILES: C[C@]1(O)C[C@@H](Nc2nnc(-c3ccc(C(F)(F)F)cc3O)c3ccncc23)C1]

### 2-(1-((1s,3s)-3-Hydroxy-3-methylcyclobutyl)amino)pyrido[3,4-d]pyridazin-4-yl)-5-trifluoromethyl)phenol

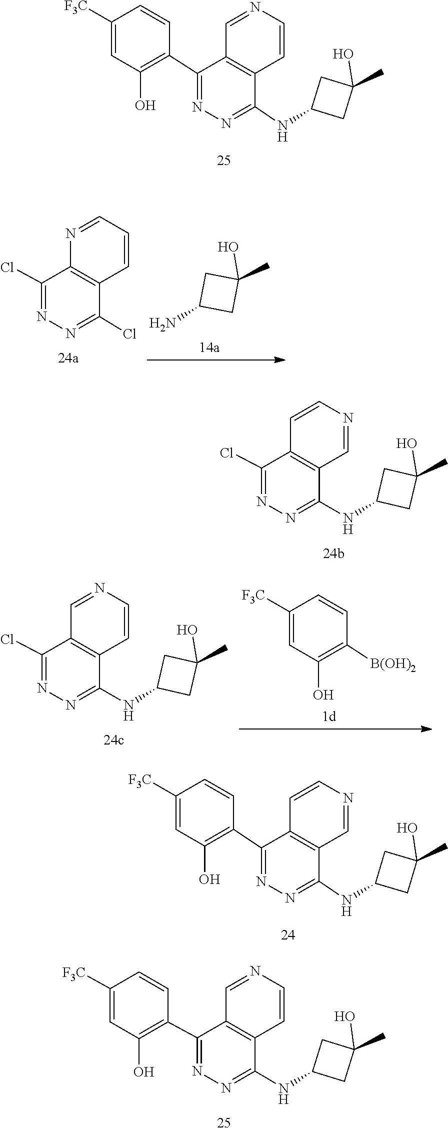

Compounds 24 and 25 were prepared by using the method described in Example 15 and using compound 24a as the starting material.

### Compound 24

¹H NMR: (400 MHz,CD₃OD) δ ppm 9.70 (br s, 1H), 8.84 (d, J=6.0 Hz, 1H), 7.58 (d, J=7.6 Hz, 1H), 7.47 (d, J=5.6 Hz, 1H), 7.32 (d, J=8.0 Hz, 1H), 7.26 (s, 1H), 4.39-4.31 (m, 1H), 2.72-2.67 (m, 2H), 2.29-2.24 (m, 2H), 1.46 (s, 3H).

### Compound 25

¹H NMR: (400 MHz,CD₃OD) δ ppm 8.91 (m, 2H), 8.21 (d, J=5.6 Hz, 1H), 7.62 (d, J=8.0 Hz, 1H), 7.34 (d, J=8.0 Hz, 1H), 7.28 (s, 1H), 4.35-4.27 (m, 1H), 2.71-2.66 (m, 2H), 2.28-2.23 (m, 2H), 1.45 (s, 3H).

### Example 24

### 2-(4-((1 s,3s)-3-Hydroxy-3-methylcyclobutyl)amino)-5,7-dihydrofuro[3,4-d]pyridazin-1-yl)-5-(trifluoromethyl)phenol

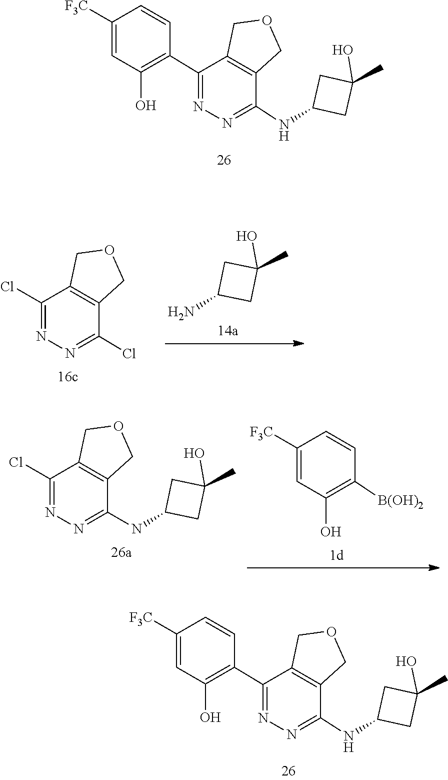

Compound 26 was prepared by using the method described in Example 16 and using compound 16c as the starting material.

¹H NMR (400 MHz, CD₃OD) Shift=7.42 (d, J=8.4 Hz, 1H), 7.21-7.20 (m, 2H), 5.39 (t, J=3.2 Hz, 2H), 5.05 (t, J=3.2 Hz, 2H), 4.19 (m, 1H), 2.61 (m, 2H), 2.14 (m, 2H), 1.43 (s, 3H).

### Example 25

### (R₂-(4-Methyl-6-((1-methylpiperidin-3-yl)amino)pyridazin-3-yl)-5,6,7,8-tetrahydronaphthalen-1-ol

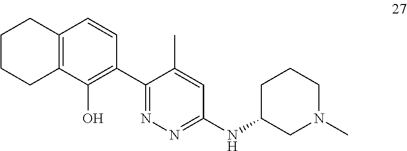 [SMILES: Cc1cc(N[C@@H]2CCCN(C)C2)nnc1-c1ccc2c(c1O)CCCC2]

### Step 1: Synthesis of (compound 27b

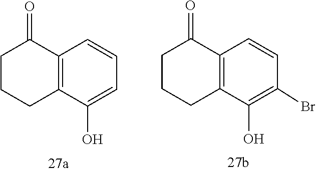

Compound 27a (1.0 g, 6.17 mmol) was dissolved in DMF (6 mL), and NBS (0.99 g, 5.55 mmol) was added. The mixture was stirred at room temperature until the reaction was complete. The reaction was diluted by addition of dichloromethane (10 mL) and saturated sodium chloride solution (10 mL). The organic phase was separated, washed with water, dried over anhydrous sodium sulfate and then filtered. The filtrate was concentrated in vacuo to give a crude product. The crude product was purified by reversed-phase flash column chromatography (eluent: 0-60% acetonitrile in water) to give compound 27b (680 mg, yield: 45.7%).

### Step 2: Synthesis of (compound 27c

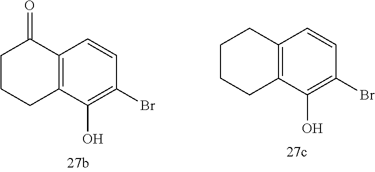 [SMILES: O=C1CCCc2c1ccc(Br)c2O ; Oc1c(Br)ccc2c1CCCC2]

Compound 27b (300 mg, 1.24 mmol) was dissolved in a mixed solvent of trifluoroacetic acid ( ) and triethylsilane ( ), and the mixture was stirred at 90° C. until the reaction was complete. The mixture was directly concentrated by rotary evaporation to remove the solvent and diluted by addition of dichloromethane (10 mL) and saturated sodium bicarbonate solution (10 mL). The organic phase was separated, washed with saturated sodium chloride solution, dried over anhydrous sodium sulfate and then filtered. The filtrate was concentrated in vacuo to give a crude product. The crude product was purified by flash column chromatography (eluent: 10% dichloromethane in petroleum ether) to give compound 27c (200 mg, yield: 70.8%).

### Step 3: Synthesis of (compound 27

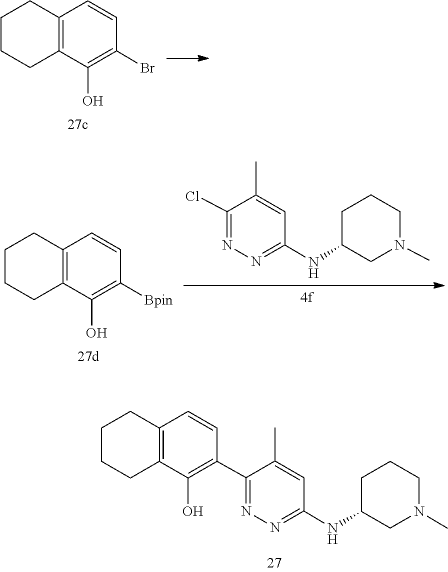

Compound 27 was prepared by using the method described in Example 4 and using compound 27c as the starting material.

¹H NMR (400 MHz, CD₃OD) Shift=7.00 (d, J=8.0 Hz, 1H), 6.81 (s, 1H), 6.69 (d, J=8.0 Hz, 1H), 4.18 (m, 1H), 2.96 (m, 1H), 2.77 (t, J=5.6 Hz, 2H), 2.69 (t, J=5.8 Hz, 2H), 2.56 (m, 5H), 2.20 (m, 4H), 2.03 (m, 1H), 1.98 (m, 1H), 1.85-1.79 (m, 5H), 1.61 (m, 2H).

### Example 26

### 5-Cyclopropyl-2-(6-((1 s,3s)-3-hydroxy-3-methylcyclobutyl)amino)-4-methylpyridazin-3-yl)phenol

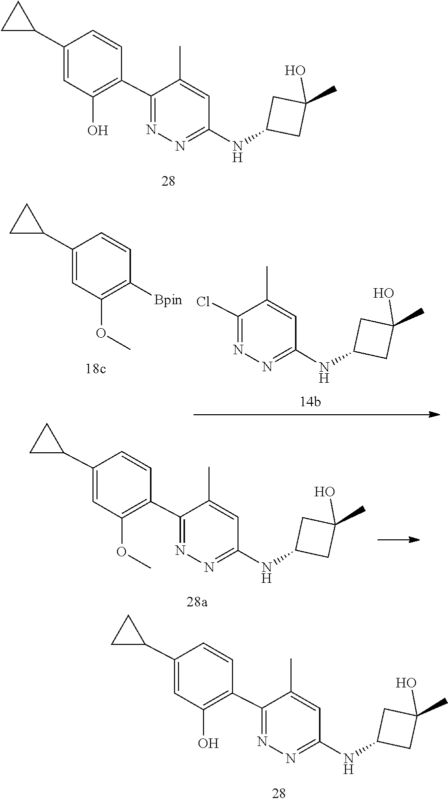

Compound 28 was prepared by using the method described in Example 18 and using compound 18c as the starting material.

LCMS: t_{R}=2.657 min in 5-95AB_7 min 220&254_Agilent.M ES-MS m/z 326.1 [M+H]⁺.

¹H NMR (400 MHz, CD₃OD) Shift=7.10 (d, J=7.6 Hz, 1H), 6.74 (s, 1H), 6.69 (d, J=7.6 Hz, 1H), 6.63 (s, 1H), 4.05-3.98 (m, 1H), 2.62-2.57 (m, 2H), 2.16 (s, 3H), 2.08-2.03 (m, 2H), 1.91-1.87 (m, 1H), 1.41 (s, 3H), 1.01-0.98 (m, 2H), 0.72-0.69 (m, 2H).

### Example 27

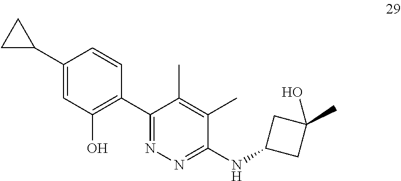 [SMILES: Cc1c(N[C@H]2C[C@@](C)(O)C2)nnc(-c2ccc(C3CC3)cc2O)c1C]

### 5-Cyclopropyl-2-(6-((1 s,3s)-3-hydroxy-3-methylcyclobutyl)amino)-4,5-dimethylpyridazin-3-yl)phenol

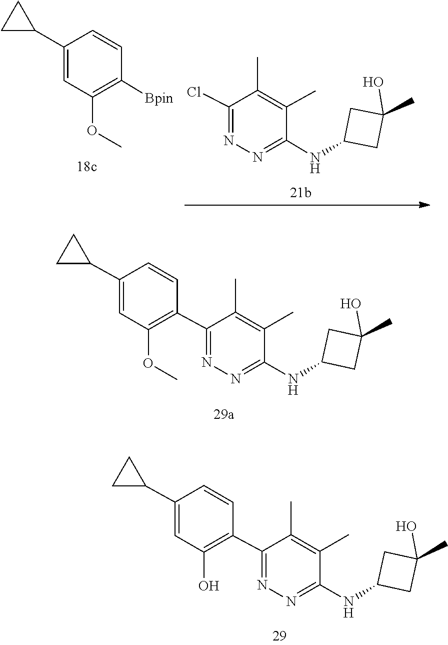

Compound 29 was prepared by using the method described in Example 18 and using compound 18c as the starting material.

LCMS: t_{R}=0.57 min in 5-95AB_1.5 min 220&254_Agilent.M ES-MS m/z 340.2 [M+H]⁺.

¹H NMR (400 MHz, CD₃OD) Shift=7.07 (d, J==7.6 Hz, 1H), 6.68 (d, J==7.6 Hz, 1H), 6.63 (s, 1H), 4.19-4.09 (m, 1H), 2.64-2.59 (m, 2H), 2.18 (s, 3H), 2.15-2.08 (m, 5H), 1.90 (m, 1H), 1.42 (s, 3H), 1.01-0.98 (m, 2H), 0.72-0.70 (m, 2H).

### Example 28

### 5_Cyclopropyl-2-(4-((1 s,3s)-3-hydroxy-3-methylcyclobutyl)amino)phthalazin-1-yl)phenol

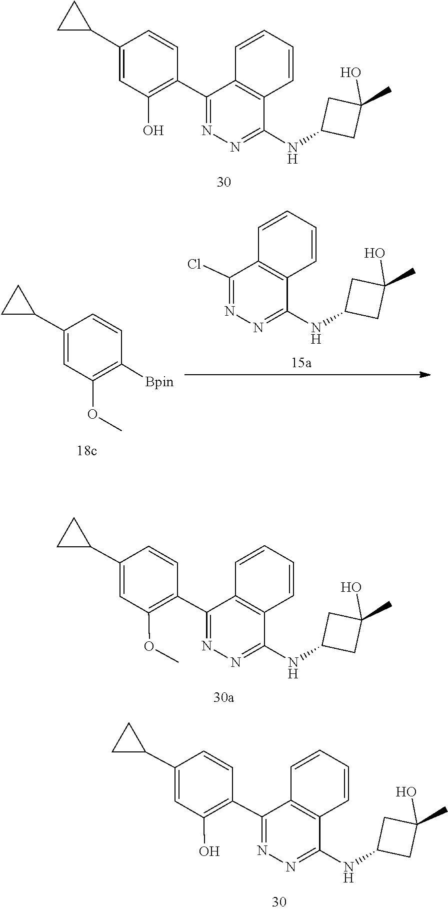

Compound 30 was prepared by using the method described in Example 18 and using compound 18c as the starting material.

LCMS: t_{R}=3.11 min in 5-95AB_7 min 220&254_Agilent.M ES-MS m/z 362.2 [M+H]⁺.

¹H NMR (400 MHz, CD₃OD) Shift=8.27 (d, J==8.0 Hz, 1H), 7.86 (t, J==7.6 Hz, 1H), 7.79 (t, J=7.6 Hz, 1H), 7.72 (d, J=8.0 Hz, 1H), 7.24 (d, J=7.6 Hz, 1H), 6.75 (d, J=8.0 Hz, 1H), 6.71 (s, 1H), 4.35-4.27 (m, 1H), 2.71-2.66 (m, 2H), 2.26-2.22 (m, 2H), 1.94 (m, 1H), 1.46 (s, 3H), 1.04-1.00 (m, 2H), 0.77-0.74 (m, 2H).

### Example 29

### 5_(4-((1s,3s)-3-Hydroxy-3-methylcyclobutyl)amino)phthalazin-1-yl)-2,3-dihydro-1H-inden-4-ol

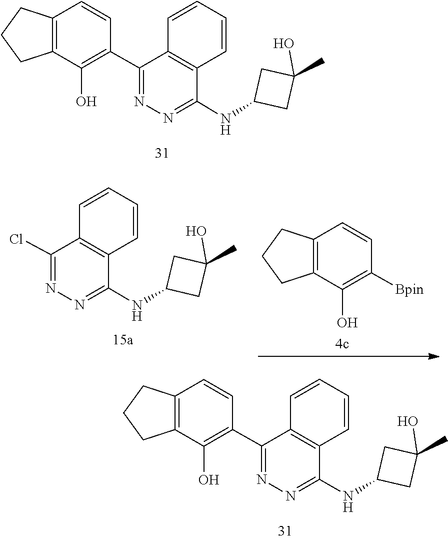

Compound 31 was prepared by using the method described in Example 14 and using compound 15a as the starting material.

LCMS: t_{R}=0.81 min in 5-95AB_1.5 min 220&254_Agilent.M ES-MS m/z 362.2 [M+H]⁺.

¹H NMR (400 MHz, DMSO-d₆) Shift=9.47 (br, 1H), 8.42 (d, J==7.6 Hz, 1H), 7.86 (t, J==7.2 Hz, 1H), 7.79 (t, J==7.2 Hz, 1H), 7.65 (d, J=7.6 Hz, 1H), 7.55 (d, J=6.4 Hz, 1H), 7.10 (d, J==7.6 Hz, 1H), 6.86 (d, J=7.6 Hz, 1H), 5.00 (s, 1H), 2.94 (t, J==7.4 Hz, 2H), 2.88 (t, J==7.4 Hz, 1H), 2.21-2.18 (m, 2H), 2.10-2.06 (m, 2H), 3.16 (br, 4H).

### Example 30

### rac-(R)-2-(4-(1-(2-Hydroxyethyl)piperidin-3-yl)amino)-5,7-dihydrofuro[3,4-d]pyridazin-1-yl)-5-(trifluoromethyl)phenol

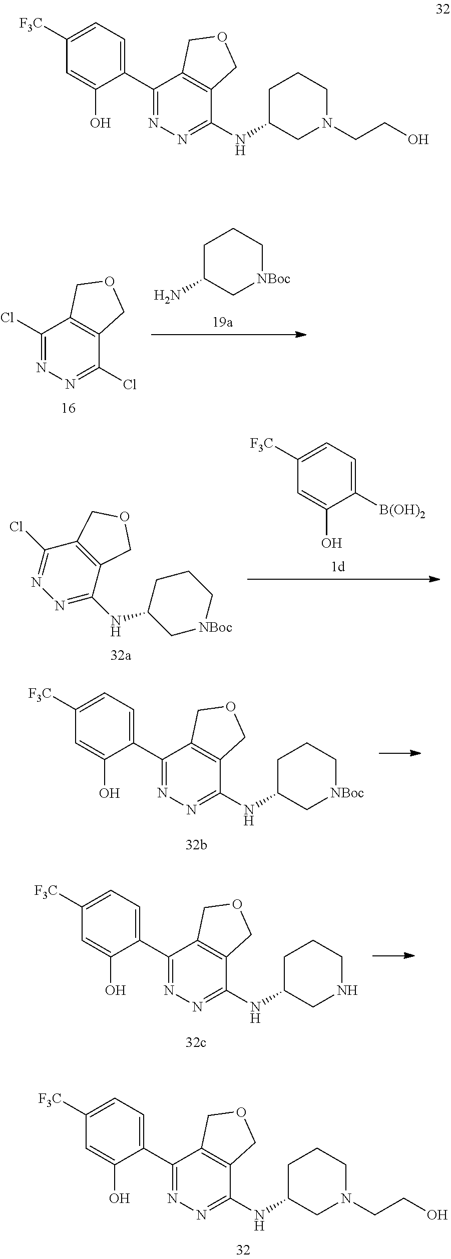

Compound 32 was prepared by using the method described in Example 19 and using compound 16c as the starting material.

¹H NMR (400 MHz, CD₃OD) δ=8.50 (s, 1H), 7.43 (d, J=8.5 Hz, 1H), 7.27-7.16 (m, 2H), 5.36 (t, J=3.0 Hz, 2H), 5.05 (br s, 2H), 4.55-4.44 (m, 1H), 3.80 (t, J=5.5 Hz, 2H), 3.50 (br d, J=10.8 Hz, 1H), 3.13 (br s, 1H), 3.01-2.83 (m, 2H), 2.76-2.64 (m, 1H), 2.59 (br s, 1H), 2.09 (br d, J=8.8 Hz, 1H), 2.03-1.93 (m, 1H), 1.92-1.79 (m, 1H), 1.74-1.55 (m, 1H).

### Example 31

### 3-Fluoro-2-(4-((1 s,3s)-3-hydroxy-3-methylcyclobutyl)amino)pyrido[3,4-d]pyridazin-1-yl)-5-(trifluoromethyl)phenol (compound 33

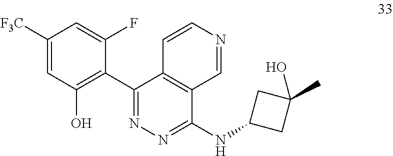 [SMILES: C[C@]1(O)C[C@@H](Nc2nnc(-c3c(O)cc(C(F)(F)F)cc3F)c3ccncc23)C1]

### 3-Fluoro-2-(1-((1 s,3s)-3-hydroxy-3-methylcyclobutyl)amino)pyrido[3,4-d]pyridazin-4-yl)-5-(trifluoromethyl)phenol (compound 34

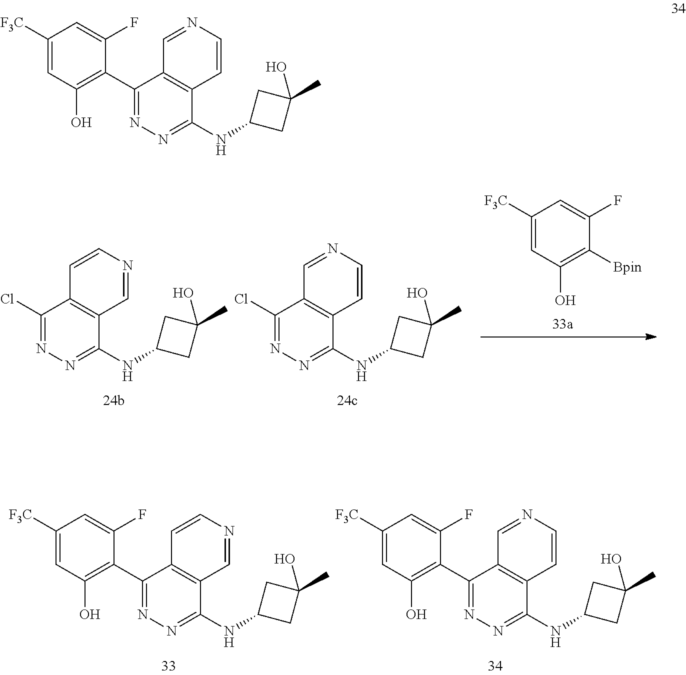

Compounds 33 and 34 were prepared by using the method described in Example 15 and using compounds 24b and 24c as the starting materials.

### Compound 33

¹H-NMR: (400 MHz, METHANOL-d4) Shift=9.71 (s, 1H), 8.84 (d, J=5.7 Hz, 1H), 7.38 (d, J=5.5 Hz, 1H), 7.05 (s, 1H), 7.01 (br d, J=8.8 Hz, 1H), 4.36 (quin, J=7.8 Hz, 1H), 2.78-2.61 (m, 2H), 2.34-2.20 (m, 2H), 1.45 (s, 3H).

### Compound 34

¹H-NMR: (400 MHz, METHANOL-d4) Shift=8.93 (d, J=5.8 Hz, 1H), 8.83 (s, 1H), 8.24 (d, J=6.1 Hz, 1H), 7.12 (br s, 2H), 4.34 (br d, J=8.3 Hz, 1H), 2.75-2.64 (m, 3H), 2.28 (br d, J=8.6 Hz, 3H), 1.45 (s, 3H).

### Example 32

### 3-Fluoro-2-(4-((R)-1-methylpiperidin-3-yl)amino)-5,7-dihydrofuro[3,4-d]pyridazin-1-yl)-5-(trifluoromethyl)phenol

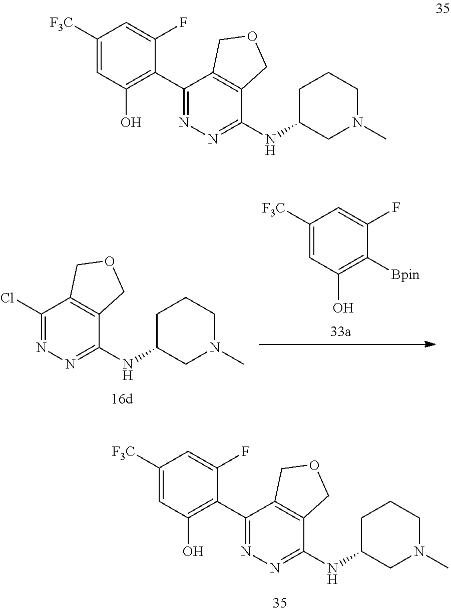

Compound 35 was prepared by using the method described in Example 15 and using compound 16d as the starting material.

¹H-NMR: (400 MHz, CD₃OD) δ=7.08-6.96 (m, 2H), 5.10-5.04 (m, 2H), 4.65-4.56 (m, 2H), 3.68-3.60 (m, 1H), 3.01-2.86 (m, 1H), 2.56-2.45 (m, 3H), 2.43-2.30 (m, 1H), 2.15-2.01 (m, 1H), 1.97-1.90 (m, 1H), 1.86-1.74 (m, 1H), 1.65-1.53 (m, 1H), 1.40-1.24 (m, 2H).

### Example 33

### rac-(R)-5-Cyclopropyl-2-(6-((1-(2-hydroxyethyl)piperidin-3-yl)amino)-4-methylpyridazin-3-yl)phenol

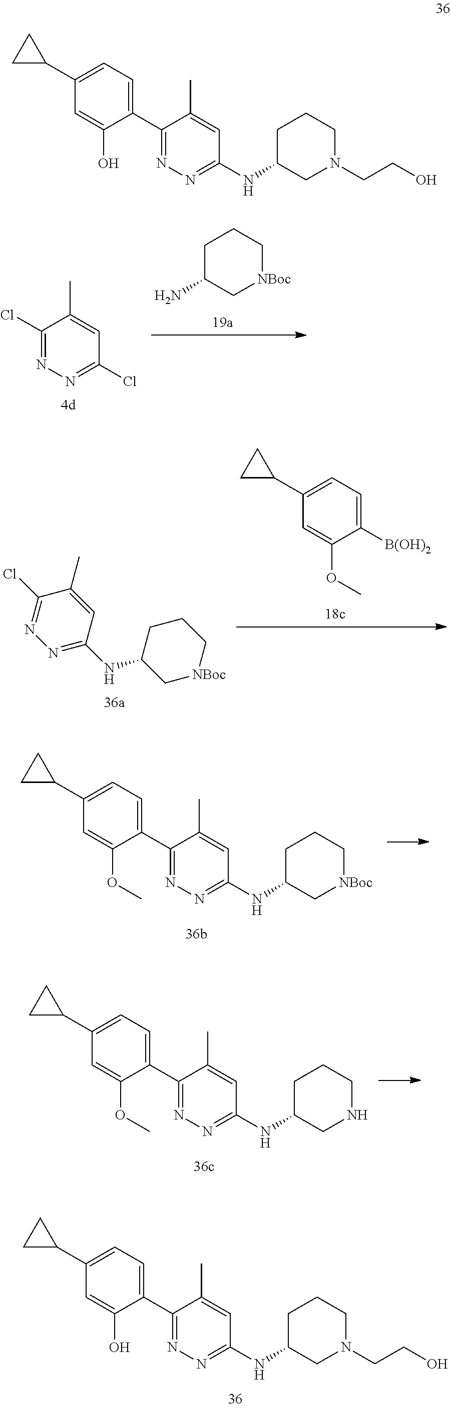

Compound 36 was prepared by using the method described in compound 19 and using compounds 4d and 19a as the starting materials.

¹H NMR: (400 MHz, CD₃OD) δ=8.50 (s, 1H), 7.88 (s, 1H), 7.63 (d, J=8.3 Hz, 1H), 6.65 (d, J=8.0 Hz, 1H), 6.62 (s, 1H), 4.52-4.40 (m, 1H), 3.83 (t, J=5.4 Hz, 2H), 3.57 (br d, J=10.0 Hz, 1H), 3.28-3.19 (m, 1H), 3.12-2.95 (m, 2H), 2.83 (br t, J=10.4 Hz, 1H), 2.75 (br t, J=10.8 Hz, 1H), 2.28 (s, 3H), 2.09 (br d, J=12.0 Hz, 1H), 2.05-1.97 (m, 1H), 1.95-1.83 (m, 2H), 1.79-1.67 (m, 1H), 1.03-0.94 (m, 2H), 0.74-0.67 (m, 2H).

### Example 34

### 7-Fluoro-5-(6-((1 s,3s)-3-hydroxy-3-methylcyclobutyl)amino)-methylpyridazin-3-yl)-2,3-dihydro-1H-inden-4-ol

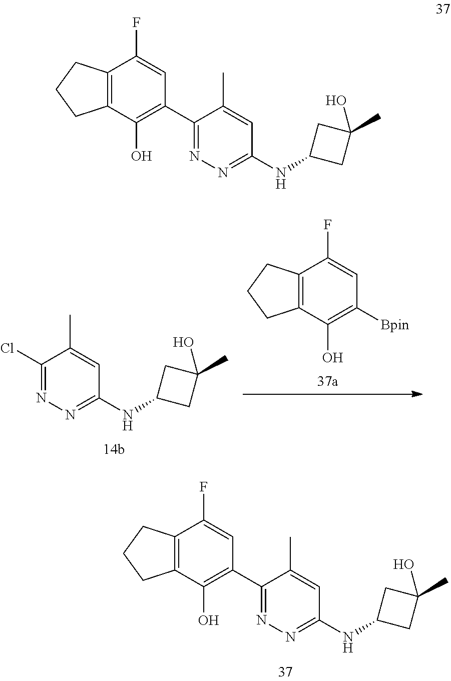

Compound 37 was prepared by using the method described in Example 4 and using compound 14b as the starting material.

¹H NMR: (400 MHz, CD₃OD) δ=6.79 (d, J=0.2 Hz, 1H), 6.77 (s, 1H), 4.01 (penta, J=7.8 Hz, 1H), 2.99 (t, J=7.6 Hz, 2H), 2.94 (t, J=7.6 Hz, 2H), 2.61-2.58 (m, 2H), 2.21-2.18 (m, 5H), 2.06-2.03 (m, 2H), 1.42 (s, 3H).

### Example 35

### 5-(8-((1s,3s)-3-hydroxy-3-methylcyclobutyl)amino)pyrido[2,3-d]pyridazin-5-yl)-2,3-dihydro-1H-inden-4-ol

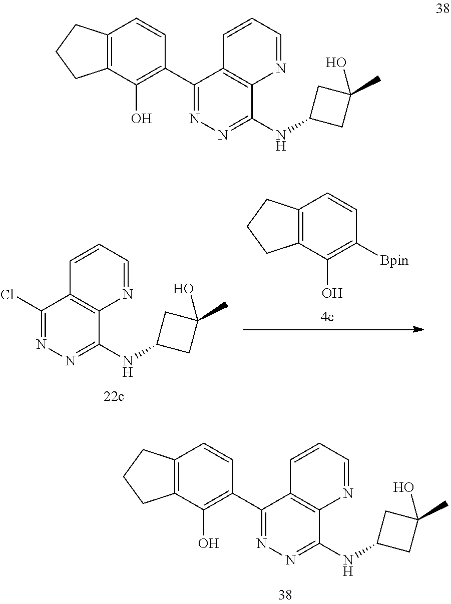

Compound 38 was prepared by using the method described in Example 15 and using 22c as the starting material.

¹H NMR: (400 MHz, DMSO-d₆) δ=9.39 (br, 1H), 9.10 (dd, J=8.4, 1.2 Hz, 1H), 8.02 (d, J=8.4 Hz, 1H), 7.86 (d, J=8.4 Hz, 1H), 7.52 (d, J=7.2 Hz, 1H), 7.14 (d, J=7.6 Hz, 1H), 6.88 (d, J=7.6 Hz, 1H), 5.00 (s, 1H), 4.36-4.26 (m, 1H), 2.93 (t, J=7.2 Hz, 2H), 2.88 (t, J=7.2 Hz, 2H), 2.32-2.22 (m, 2H), 2.18-2.08 (m, 2H), 1.33 (s, 3H).

### Example 36

### 5-(4-((l s,3s)-3-Hydroxy-3-methylcyclobutyl)amino)pyrido[3,4-d]pyridazin-1-yl)-2,3-dihydro-1H-inden-4-ol (compound 39

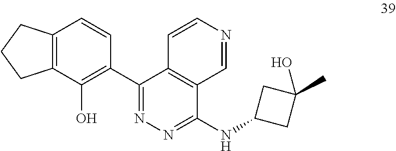 [SMILES: C[C@]1(O)C[C@@H](Nc2nnc(-c3ccc4c(c3O)CCC4)c3ccncc23)C1]

### 5-(1-((1s,3s)-3-Hydroxy-3-methylcyclobutyl)amino)pyrido[3,4-d]pyridazin-1-yl)-2,3-dihydro-1H-inden-4-ol (compound 40

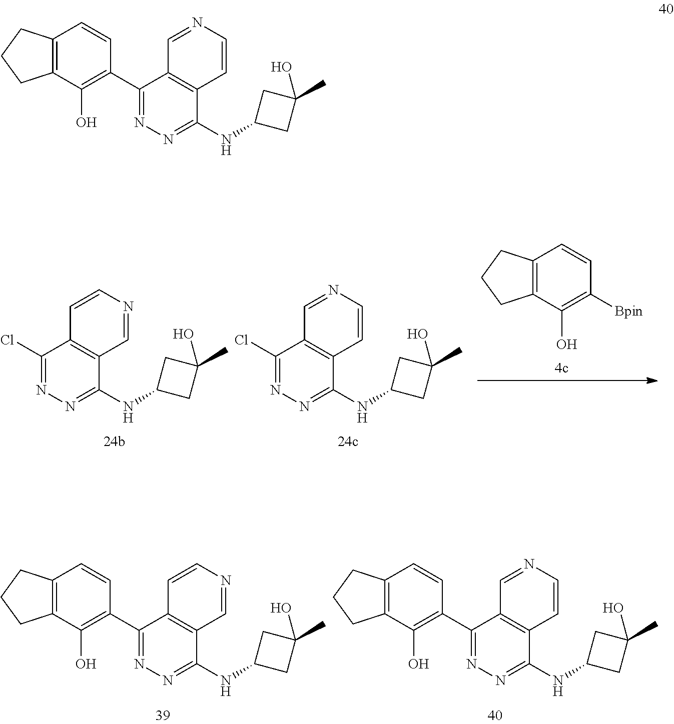

Compounds 39 and 40 were prepared by using the method described in Example 15 and using 24b and 24c as the starting materials.

### Compound 39

¹H NMR: (400 MHz,CD₃OD) δ ppm 9.05 (br s, 1H), 8.90 (d, J=5.6 Hz, 1H), 8.21 (d, J=5.6 Hz, 1H), 7.24 (d, J=7.6 Hz, 1H), 6.97 (d, J=7.6 Hz, 1H), 4.01 (penta, J=7.8 Hz, 1H), 3.01 (t, J=7.6 Hz, 2H), 2.70 (t, J=7.6 Hz, 2H), 2.70-2.61 (m, 2H), 2.26-2.17 (m, 4H), 1.42 (s, 3H).

### Compound 40

¹H NMR: (400 MHz,CD₃OD) δ ppm 9.69 (s, 1H), 8.85 (d, J=5.6 Hz, 1H), 7.64 (d, J=5.6 Hz, 1H), 7.21 (d, J=7.6 Hz, 1H), 6.92 (d, J=7.6 Hz, 1H), 4.35 (penta, J=7.8 Hz, 1H), 3.01 (t, J=7.6 Hz, 2H), 2.96 (t, J=7.6 Hz, 2H), 2.71-2.61 (m, 2H), 2.28-2.16 (m, 4H), 1.47 (s, 3H).

### Example 37

### 5-Cyclopropyl-2-(4-((1s,3s)-3-hydroxy-3-methylcyclobutyl)amino)pyrido[3,4-d]pyridazin-1-yl)phenol

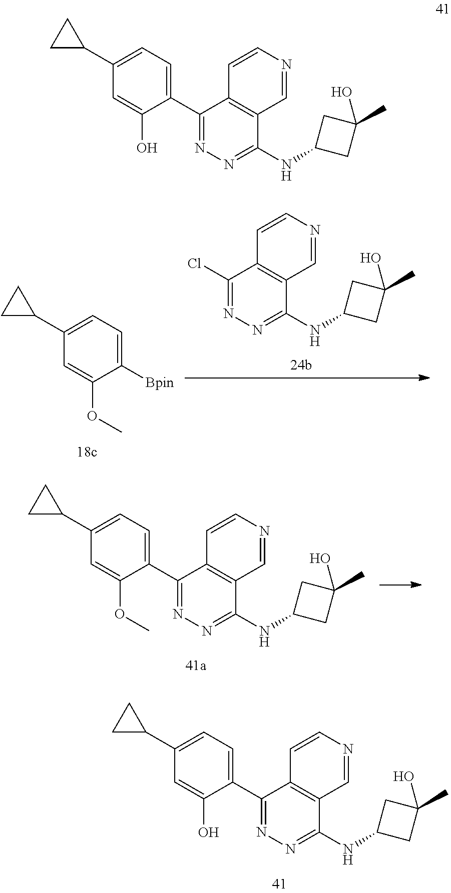

Compound 41 was prepared by using the method described in compound 18 and using compound 18c as the starting material.

¹H NMR (400 MHz, CD₃OD) δ ppm 9.70 (s, 1H), 8.86 (d, J=5.7 Hz, 1H), 7.57 (d, J=5.6 Hz, 1H), 7.28 (d, J=7.8 Hz, 1H), 6.79 (br d, J=7.8 Hz, 1H), 6.73 (s, 1H), 5.43-5.43 (m, 1H), 4.34 (quin, J=7.8 Hz, 1H), 2.76-2.65 (m, 2H), 2.35-2.23 (m, 2H), 2.02-1.90 (m, 1H), 1.08-0.99 (m, 2H), 0.81-0.70 (m, 2H).

### Example 38

### 2-(4-((l s,3s)-3-Hydroxy-3-methylcyclobutyl)amino)-7,8-dihydro-5H-pyrano[3,4-d]pyridazin-1-yl)-5-trifluoromethylphenol (compound 42

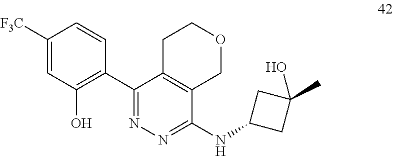 [SMILES: C[C@]1(O)C[C@@H](Nc2nnc(-c3ccc(C(F)(F)F)cc3O)c3c2COCC3)C1]

### 2-(1-((1s,3s)-3-Hydroxy-3-methylcyclobutyl)amino)-7,8-dihydro-5H-pyrano[3,4-d]pyridazin-4-yl)-5-(trifluoromethyl)phenol (compound 43

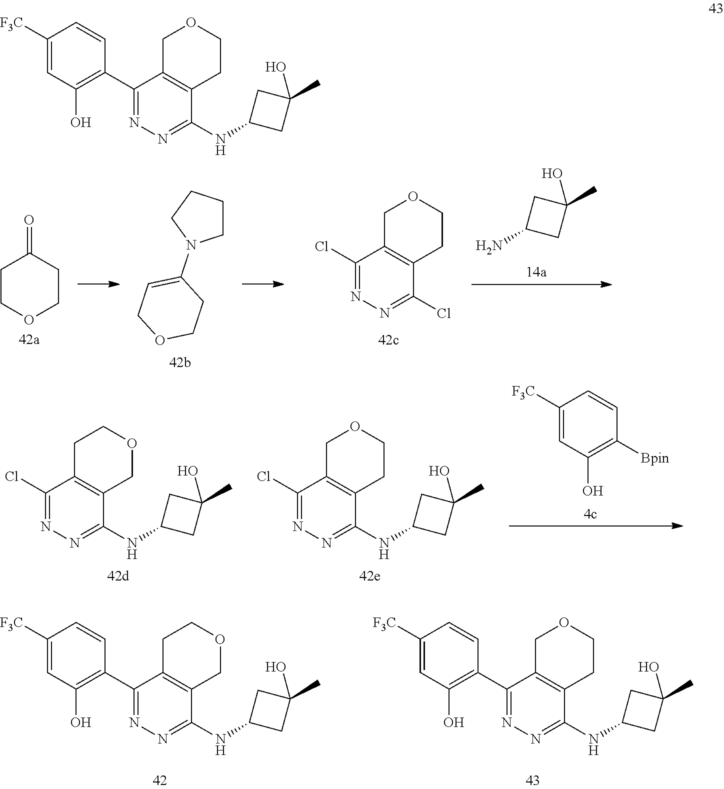

Compounds 42 and 43 were prepared by using the method described in Example 15 and using 42a as the starting material.

### Compound 42

¹H NMR: (400 MHz,CD₃OD) δ ppm 7.40 (d, J=7.6 Hz, 1H), 7.24 (d, J=7.6 Hz, 1H), 4.48 (s, 2H), 4.21 (penta, J=7.6 Hz, 1H), 4.02 (t, J=7.6 Hz, 2H), 2.68-2.56 (m, 4H), 2.16-2.02 (m, 2H), 1.43 (s, 3H).

### Compound 43

¹H NMR: (400 MHz,CD₃OD) δ ppm 7.42 (d, J=7.6 Hz, 1H), 7.23 (d, J=7.6 Hz, 1H), 4.61 (s, 2H), 4.19 (penta, J=7.6 Hz, 1H), 3.89 (t, J=7.6 Hz, 2H), 2.70-2.58 (m, 4H), 2.14-2.00 (m, 2H), 1.41 (s, 3H).

### Example 39

### 3-Fluoro-2-(4-((l s,3s)-3-hydroxy-3-methylcyclobutyl)amino)phthalazin-1-yl)-5-(trifluoromethyl)phenol

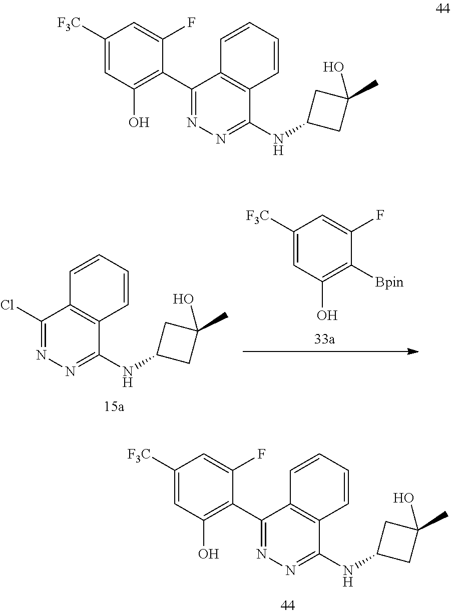

Compound 44 was prepared by using the method described in Example 15 and using compound 15a as the starting material.

¹H-NMR: (400 MHz, DMSO-d₆) δ=10.78 (br, 1H), 8.48 (d, J=8.0 Hz, 1H), 7.90 (t, J=7.8 Hz, 1H), 7.81 (t, J=7.7 Hz, 1H), 7.36 (d, J=8.0 Hz, 1H), 7.27 (d, J=7.6 Hz, 1H), 7.17 (s, 1H), 5.05 (s, 1H), 4.28-4.22 (m, 1H), 2.28-2.20 (m, 2H), 1.34 (s, 3H).

### Example 40

### 2-(4-((1s,3s)-3-Hydroxy-3-methylcyclobutyl)amino)pyrido[3,4-d]pyridazin-1-yl)-3-ethyl-5-(trifluoromethyl)phenol

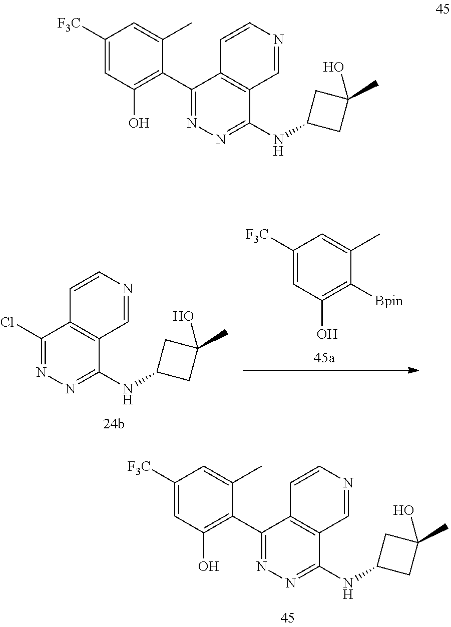

Compound 45 was prepared by using the method described in Example 15 and using compound 24b as the starting material.

¹H-NMR: (400 MHz, DMSO-d₆) δ=9.81 (s, 1H), 8.81 (d, J=5.2 Hz, 1H), 8.08 (d, J=6.4 Hz, 1H), 7.20 (s, 1H), 7.13-7.03 (m, 2H), 5.05 (br s, 1H), 4.49-4.15 (m, 1H), 2.52-2.52 (m, 2H), 2.26 (br t, J=0.2 Hz, 1H), 2.16 (br t, J=10.0 Hz, 1H), 1.99 (s, 3H), 1.35 (s, 3H).

### Example 41

### 2-(4-((1 s,3s)-3-Hydroxy-3-methylcyclobutyl)amino)phthalazin-1-yl)-3-methyl-5-(trifluoromethyl)phenol

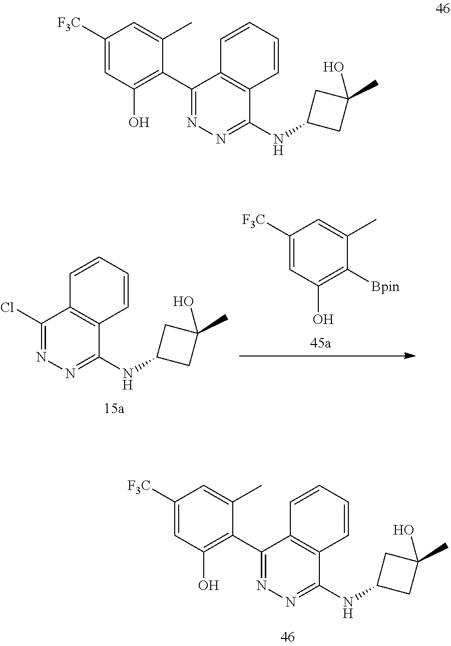

Compound 46 was prepared by using the method described in Example 15 and using compound 15a as the starting material.

¹H-NMR: (400 MHz, CD₃OD) δ=8.39 (d, J=8.4 Hz, 1H), 7.97-7.87 (m, 1H), 7.86-7.78 (m, 1H), 7.42 (d, J=8.0 Hz, 1H), 7.17 (s, 1H), 7.08 (s, 1H), 4.39-4.22 (m, 1H), 2.76-2.63 (m, 2H), 2.36-2.20 (m, 2H), 2.06 (s, 3H), 1.45 (s, 3H).

### Example 42

### 3-Methyl-2-(4-((R)-1-methylpiperidin-3-yl)amino)-5,7-dihydrofuro[3,4-d]pyridazin-1-yl)-5-(trifluoromethyl)phenol

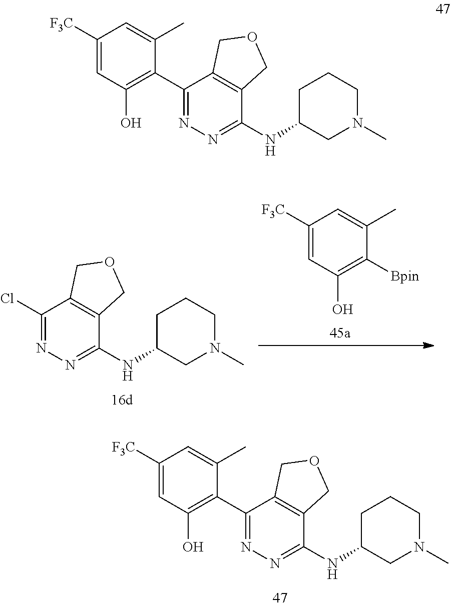

Compound 47 was prepared by using the method described in Example 15 and using compound 16d as the starting material.

¹H-NMR: (400 MHz, DMSO-d₆) δ=8.16 (s, 1H), 7.12 (s, 1H), 7.05 (s, 1H), 6.50 (br d, J=6.9 Hz, 1H), 4.95 (br s, 2H), 4.81 (br s, 1H), 4.69 (br s, 1H), 4.29 (br s, 1H), 3.13-3.01 (m, 1H), 2.78-2.63 (m, 1H), 2.26 (s, 3H), 2.08 (s, 3H), 1.94 (br d, J=12.8 Hz, 2H), 1.75 (br d, J=13.9 Hz, 1H), 1.59 (br d, J=12.1 Hz, 1H), 1.34 (br d, J=11.4 Hz, 1H).

### Example 43

### 3-Fluoro-2-(4-((R)-1-methylpiperidin-3-yl)amino)-6,7-dihydro-5H-cyclopenta[d]pyridazin-1-yl)-5-(trifluoromethyl)phenol

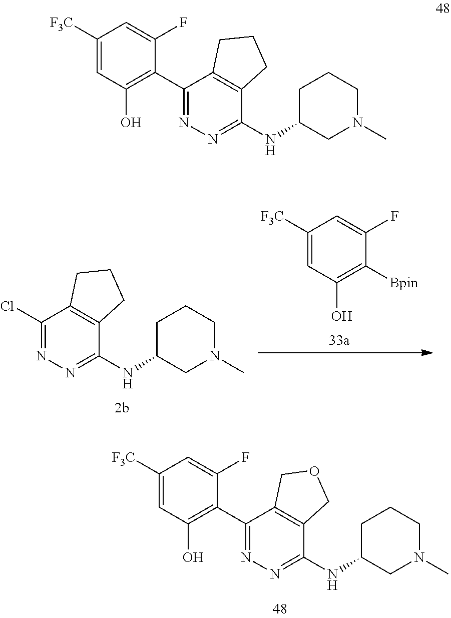

Compound 48 was prepared by using the method described in Example 15 and using compound 2b as the starting material.

LCMS: ES-LCMS m/z 411.3 [M+H]⁺.

### Example 44

### 3-Fluoro-2-(4-((R)-1-(2-hydroxyethyl)piperidin-3-yl)amino)-5,7-dihydrofuro[3,4-d]pyridazin-1-yl)-5-(trifluoromethyl)phenol

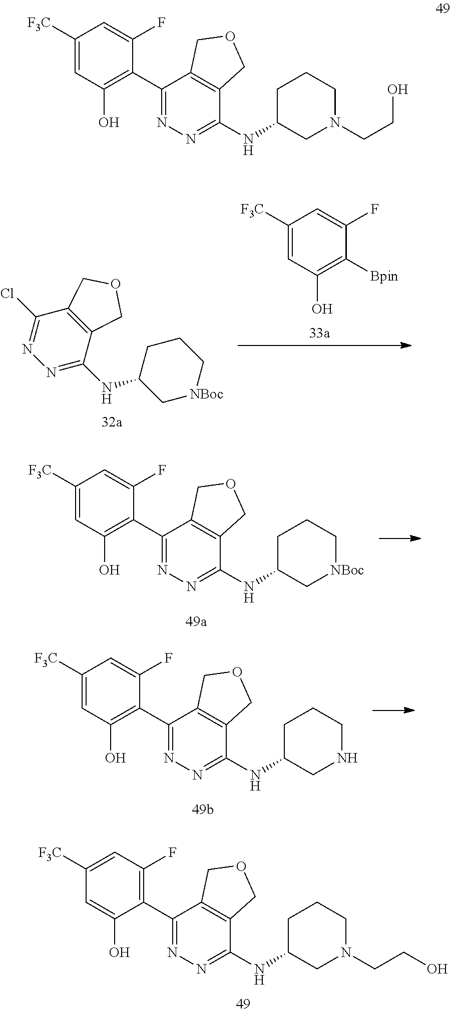

Compound 49 was prepared by using the method described in Example 19 and using compound 32a as the starting material.

LCMS: ES-LCMS m/z 443.3 [M+H]⁺.

### Example 45

### (R)-2-(4-((1-Methylpiperid in-3-yl)amino)-7,8-dihydro-5H-pyrano[3,4-d]pyridazin-1-yl)-5-(trifluoromethyl)phenol (compound 50

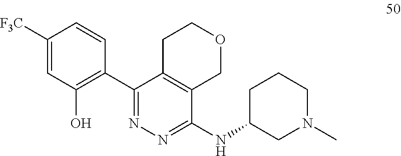 [SMILES: CN1CCC[C@@H](Nc2nnc(-c3ccc(C(F)(F)F)cc3O)c3c2COCC3)C1]

### (R)-2-(1-((1-Methylpiperid in-3-yl)amino)-7,8-dihydro-5H-pyrano[3,4-d]pyridazin-4-yl)-5-trifluoromethyl)phenol (compound 51

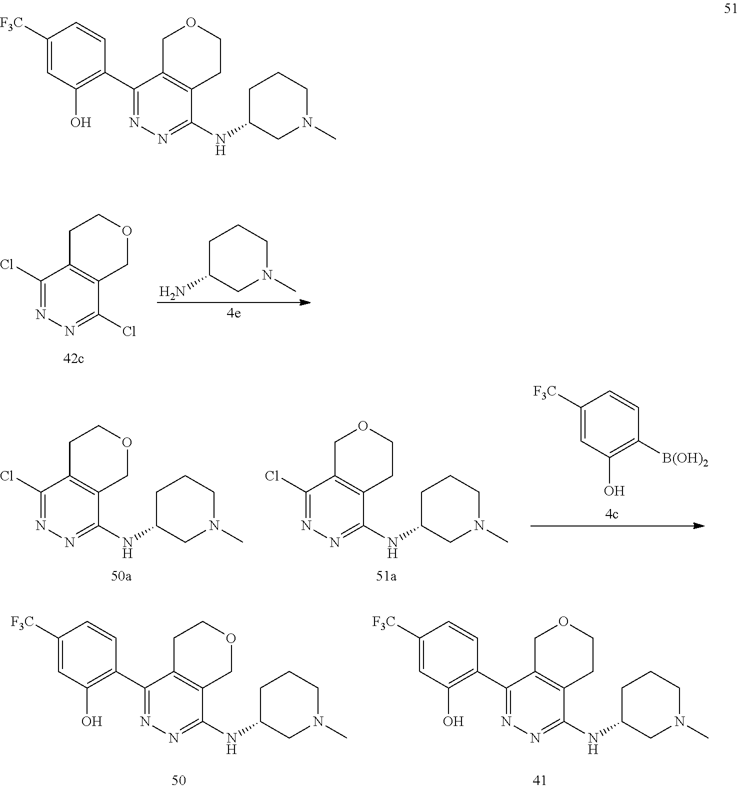

Compounds 50 and 51 were prepared by using the method described in compound 16 and using compound 42c as the starting material.

Compound 50: LCMS: ES-LCMS m/z 409.3 [M+H]⁺.

Compound 51: LCMS: ES-LCMS m/z 409.3 [M+H]⁺.

### Example 46

### (R)-2-(4-(1-(2-Hydroxyethyl)piperidin-3-yl)amino)-7,8-dihydro-5H-pyrano[3,4-d]pyridazin-1-yl)-5-(trifluoromethyl)phenol (compound 52

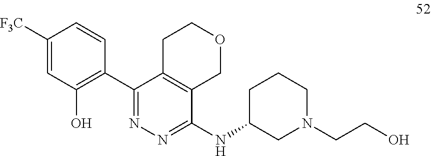 [SMILES: OCCN1CCC[C@@H](Nc2nnc(-c3ccc(C(F)(F)F)cc3O)c3c2COCC3)C1]

### (R)-2-(1-(2-Hydroxyethyl)piperid in-3-yl)amino)-7,8-dihydro-5H-pyrano[3,4-d]pyridazin-4-yl)-5-(trifluoromethyl)phenol (compound 53

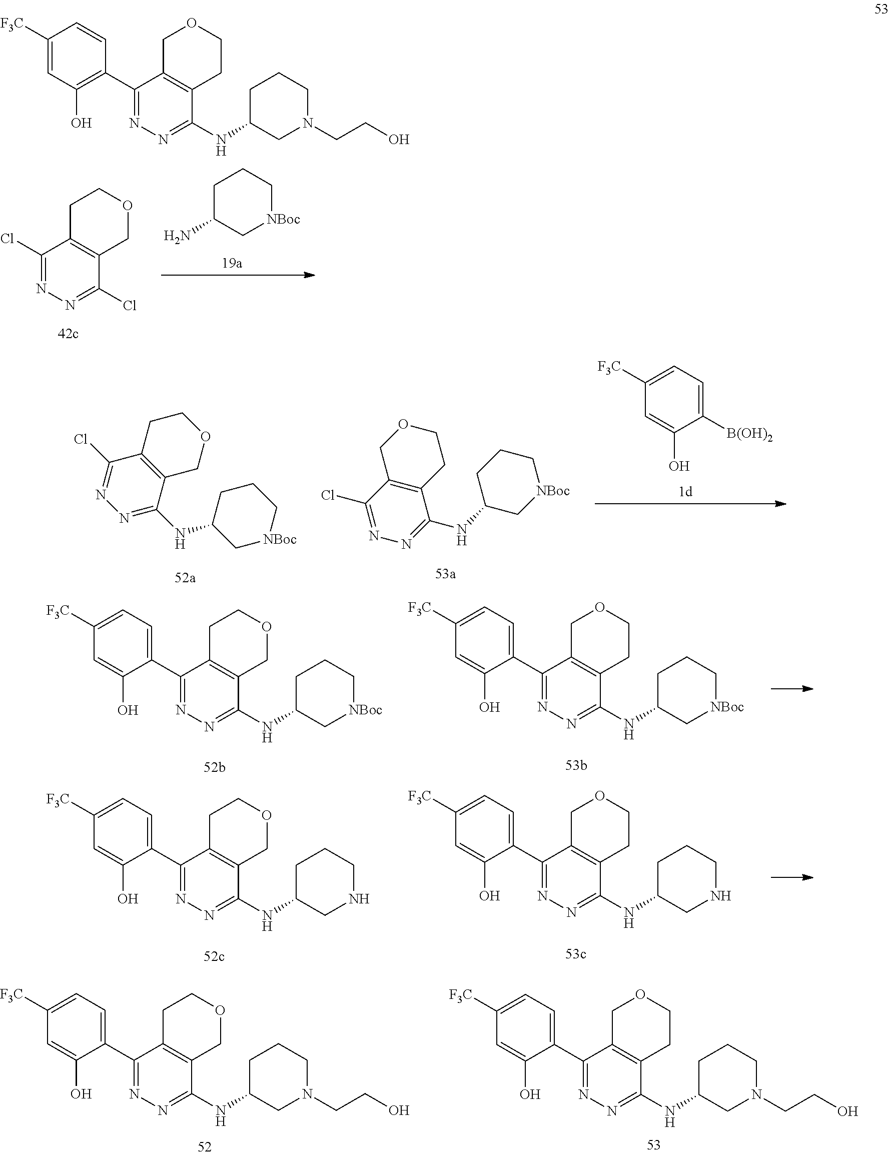

Compounds 52 and 53 were prepared by using the method described in compound 32 and using compound 16c as the starting material.

Compound 52: LCMS: ES-LCMS m/z 439.3 [M+H]⁺.

Compound 53: LCMS: ES-LCMS m/z 439.3 [M+H]⁺.

### Biological Evaluation

The present disclosure is further described and explained below with reference to test examples. However, these test examples are not intended to limit the scope of the present disclosure.

### Experimental Example 1. Assays for Inhibitory Activity Against NLRP3 Inflammasome in Human Monocytes

### 1. Experimental Instruments and Reagents

### 1.1. Experimental Instruments

Plate washer: BioTek 405 Select 405TSUS Microplate Washer 96 and 384 Well w/Ultrasonic (6025) (BioTek, cat #405TSUS)
Plate reader: PerkinElmer 2104 EnVision Multilabel Plate Readers

### 1.2. Experimental Reagents

| Reagent | Supplier |
|---|---|
| Human IL-1b ELISA kit | BD |
| Penicillin/streptomycin | Gibco |
| RPMI1640 medium | Gibco |
| HEPES | Gibco |
| FBS | Gibco |
| Lipopolysaccharide | Sigma |
| Hygromycin B | Client |
| Normocin | Client |
| ATP | Sigma |
| 96-well plate, Elisa | Greiner |
| 96-well plate | Corning |

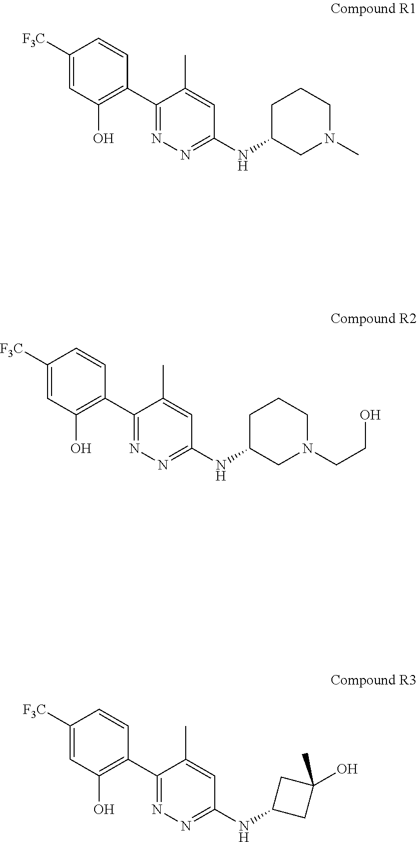 [SMILES: Cc1cc(N[C@@H]2CCCN(C)C2)nnc1-c1ccc(C(F)(F)F)cc1O ; Cc1cc(N[C@@H]2CCCN(CCO)C2)nnc1-c1ccc(C(F)(F)F)cc1O ; Cc1cc(N[C@H]2C[C@@](C)(O)C2)nnc1-c1ccc(C(F)(F)F)cc1O]

Compounds R₁, R₂ and R₃ were synthesized as reported in WO2020234715.

### 2. Experimental Protocol

Day 1: PBMCs were separated from human blood by density gradient centrifugation and washed twice with PBS containing 2% FBS (centrifuged at 300 g for 8 min). Monocytes were then isolated from the PBMCs using a human pan-monocyte isolation kit and an LS column. The cells were stained with CD₁₄-FITC at 4° C. for 30 min, and FACS was run on BD FACS Verse to analyze the purity of the pan-monocytes. The cells were counted, and the cell density was adjusted to 2.5×10⁵ cells/mL. The cells were seeded into a 96-well plate at 2.5×10⁴ monocytes/100 mL suspension/well. The plate was incubated overnight at 37° C. in 5% CO₂.

Day 2: Test compounds were pre-titrated so that all titration points, including the DMSO control well, contained 0.1% DMSO. The medium was removed, and the monocytes were pre-treated (by adding 150 mL of compound (diluted in serum-free 1640 medium) or DMSO to their respective wells and incubating the cells at 37° C. in 5% CO₂ for 0.5 h). The cells were then treated (by adding 25 mL of a 1640 (serum-free) solution containing 700 ng/mL LPS (the final concentration was 100 ng/mL) and incubating the cells at 37° C. in 5% CO₂ for 3.5 h). At the end of the 3.5 hours' incubation, the cells were stimulated (by adding 25 mL of 40 mM ATP (the final concentration would be 5 mM) to treat the cells for 45 min). 80 mL of supernatant was transferred to a new plate and stored at 80° C.

Day 3: The supernatant solution was 20-fold diluted for human monocyte IL-1b ELISA according to the manufacturer's instructions.

Days 3-4: ELISA experiment
1) Day 3: A capture antibody (diluted with coating buffer) was added to the plate at 100 mL/well. The plate was sealed and incubated overnight at 4° C.
2) Day 4: The liquids in the wells were pipetted off, and the plate was washed 3 times with wash buffer, 300 μL/≥each time. After the last wash, the plate was inverted and blotted dry with absorbent paper to remove any residual buffer.

3) A test dilution was added to the plate at ≥200 μL/well. The plate was incubated at room temperature for 1 h.
4) The plate was blotted dry/washed as in step 2.
5) Standard and sample dilutions were prepared with the test dilution.
6) Each standard, sample and control was added to a corresponding well at 100 mL/well. The plate was sealed and incubated at room temperature for 2 h.
7) The plate was blotted dry/washed as in step 2, except that it was washed 5 times.
8) The assay antibody was diluted with the test dilution and added to wells at 100 mL/well.
9) The plate was sealed and incubated at room temperature for 1 h.
10) The plate was blotted dry/washed as in step 2, except that it was washed 5 times.
11) The enzyme reagent was diluted with the test dilution and added to wells at 100 mL/well. The plate was sealed and incubated at room temperature for 30 min.
12) The plate was blotted dry/washed, and the step of soaking for 30 s-1 min was used. The plate was washed 7 times in total.
13) A substrate solution was added at 100 mL/well. The plate (without plate sealant) was incubated at room temperature in the dark for 30 min.
14) A stop solution was added at 50 mL/well.
15) The absorbance at 450 nm was read using the instrument Envision within 30 min of stopping the reaction. If a wavelength correction was available, the absorbance at 570 nm would be subtracted from the absorbance at 450 nm.

### 3. Experimental Results

| Compound No. | IL-1β inhibition (EC₅₀)/nM |
|---|---|
| 1 | 3.59* (n = 2) |
| 2 | 0.843 |
| 3 | 1.43 |
| 4 | 1.91 |
| 5 | 41.8 |
| 6 | 18.7 |
| 7 | 22.64 (n = 2) |
| 8 | 4.31 |
| 9 | 41.82 |
| 10 | 48.18 |
| 11 | 2.47 |
| 12 | / |
| 13 | 129.2 |
| 14 | 2.38 |
| 15 | 4.25 |
| 16 | 0.79 |
| 17 | 14.43 |
| 18 | 0.38 |
| 19 | 4.99 |
| 20 | 1.23 |
| 21 | 4.62 |
| 22 | 35.52 |
| 23 | 2.51 |
| 24 | 0.68 (n = 2) |
| 25 | 12.76 |
| 26 | 5.88 |
| 27 | 4.75 |
| 28 | 17.43 |
| 29 | 37.24 |
| 30 | 13.44 |
| 31 | 1.08 |
| 32 | 0.71 |
| 33 | 22 |
| 34 | 27 |
| 35 | 1.4 |
| 36 | 127 |
| 37 | 60 |
| 38 | 3.0 |
| 39 | 1.0 |
| 40 | 1.1 |
| 41 | 27 |
| 42 | 20 |
| 43 | 20 |
| 44 | 24 |
| 45 | 4.9 |
| 46 | 10.1 |
| 47 | 32.2 |
| R1 | 0.51 |
| R2 | 0.89 |
| R3 | 11.1 |

*A mean value of the results of n tests after the experimental conditions were stable.

### Experimental Example 2. Experimental Protocol for Pharmacokinetics in Rats

1. Three healthy adult SD rats, SPF, male, 6-8 weeks old; weighing 200-300 g.
2. Required equipment: HPLC-MS, an analytical balance, a scale for weighing animals, a magnetic stirrer, a refrigerated centrifuge, a manual single-channel pipette, and the like.
3. An appropriate amount of test sample was measured out and dissolved in 10% DMA/33% PEG400/57% Water (V/V/V). The solution was vortexed and ultrasonicated to prepare the desired formulation for intravenous administration. An appropriate amount of test sample was measured out and dissolved in 0.5% HPMC 0.1% Tween in water. The solution was vortexed and ultrasonicated to prepare the desired formulation for oral administration.
4. The animals are housed in a cage for rats. A fast (not less than 10 h) began the day before the experiment, and during the fast, water was accessible. The animals were weighed before administration, and doses were calculated according to body weight. The animals were dosed once by intravenous injection or oral gavage on the day of administration.
5. Blood collection time points, intravenous administration groups: 0.083, 0.25, 0.5, 1, 2, 4, 8 and 24 h after administration. Oral administration group: 0.25, 0.5, 1, 2, 4, 8 and 24 h after administration.
6. Blood was collected from the jugular vein at about 0.20 mL/time point, treated with the K₂-EDTA anticoagulant, and placed on ice after collection. Plasma was separated by centrifugation within 1 h of collecting the blood samples (centrifugation conditions: 6800 g/min, 6 min, 2-8° C.). The collected plasma samples were stored in a freezer at −80° C. before analysis, and after analysis, the residual plasma samples were put back in the 80° C. freezer.
7. Biological analysis: In plotting plasma drug concentration-time curves, BLQ was recorded as 0. When the pharmacokinetic parameters were calculated, the concentration before administration was calculated as 0; BLQ before Cmax (including “No peak”) was calculated as 0; and BLQ that occurs after Cmax (including “No peak”) was excluded from the calculation. The pharmacokinetic parameters such as AUC(0t), T1/2, Cmax, Tmax and MRT were calculated from plasma concentration data at different time points using WinNonlin.

1. A compound of formula I or a pharmaceutically acceptable salt thereof, 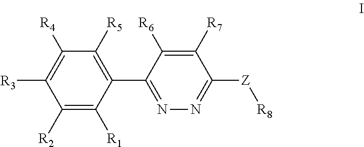
wherein R₁ is selected from the group consisting of hydrogen, deuterium, halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN;
R₂, R₃, R₄, R₅, R₆ and R₇:
(a) R₂ and R₃, together with the atoms to which they are attached, form a 5-6 membered cyclic hydrocarbon, phenyl, a heterocyclic ring or a heteroaromatic ring that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more deuterium atoms or halogens;
R₆ and R₇, together with the atoms to which they are attached, form a 5-6 membered cyclic hydrocarbon, a 5-6 membered heterocyclic ring, or 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more substituents selected from the group consisting of deuterium and halogen; R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, —S—C₁₋₆ alkyl, C₃₋₆ cycloalkyl and C₃₋₆ cycloalkylmethylene, and the substituents are selected from the group consisting of: deuterium, halogen, —OH, —NH₂ and —CN;
Z is O or —NH—(CH₂)n-, and n is an integer selected from 0-3;
R₈ is selected from the group consisting of heterocyclyl, C₃₋₈ cycloalkyl that are optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O)₂NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

2. (canceled)

3. (canceled)

4. (canceled)

5. The compound or the pharmaceutically acceptable salt thereof according to claim 1, wherein R₁ is selected from the group consisting of halogen, —OH, —NH₂, —CN, and the following groups that are optionally substituted with one or more substituents: C₁₋₆ alkyl, —O—C₁₋₆ alkyl, —NHC(═O)—C₁₋₆ alkyl and —(C═O)NH—C₁₋₆ alkyl, and the substituents are selected from the group consisting of: deuterium, halogen, —OH, —NH₂ and —CN.

6. The compound or the pharmaceutically acceptable salt thereof according to claim 5, wherein R₁ is —OH.

7. (canceled)

8. (canceled)

9. (canceled)

10. (canceled)

11. (canceled)

12. (canceled)

13. The compound or the pharmaceutically acceptable salt thereof according to any one of claim 1, wherein
R₂ and R₃, together with the atoms to which they are attached, form a 5-6 membered cyclic hydrocarbon that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more substituents selected from the group consisting of deuterium and halogen;
R₆ and R₇, together with the atoms to which they are attached, form 5-6 membered heteroaryl that is optionally substituted with one or more substituents selected from the group consisting of: deuterium, halogen, —OH, —NH₂, —CN, oxo, C₁₋₆ alkyl, —O—C₁₋₆ alkyl and C₃₋₆ cycloalkyl, and the C₁₋₆ alkyl, —O—C₁₋₆ alkyl and C₃₋₆ cycloalkyl are optionally further substituted with one or more substituents selected from the group consisting of deuterium and halogen;
R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

14. The compound or the pharmaceutically acceptable salt thereof according to claim 1, wherein R₂ and R₃, together with the atoms to which they are attached, form a 5-6 membered cyclic hydrocarbon;
R₆ and R₇, together with the atoms to which they are attached, form 5-6 membered heteroaryl; the 5-6 membered heteroaryl is optionally substituted with a substituent selected from the group consisting of hydrogen, deuterium, halogen, —OH, C₁₋₆ alkyl and C₁₋₆ haloalkyl;
R₄ and R₅ are independently selected from the group consisting of hydrogen, deuterium, halogen, —OH, —NH₂, and the following groups that are optionally substituted with one or more substituents: C₁₋₆ alkyl, —O—C₁₋₆ alkyl, C₃₋₆ cycloalkyl and C₃₋₆ cycloalkylmethylene, and the substituents are selected from the group consisting of: deuterium, halogen, —OH, —NH₂ and —CN.

15. (canceled)

16. (canceled)

17. (canceled)

18. (canceled)

19. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The compound or the pharmaceutically acceptable salt thereof according to claim 1, wherein Z is O.

50. The compound or the pharmaceutically acceptable salt thereof according to claim 1, wherein Z is —NH—(CH₂)n-, and n is an integer selected from 0-2.

51. The compound or the pharmaceutically acceptable salt thereof according to claim 1, wherein R₈ is selected from 5-10 membered heterocyclyl that is optionally substituted with one or more substituents selected from the group consisting of deuterium, halogen, oxo, —OR₈ₐ, —SR₈ₐ, —C(═O)R₈ₐ, —OC(═O)R₈ₐ, —C(═O)OR₈ₐ, —C(═O)NR₈ₐR_{8b}, —NR₈ₐR_{8b}, —NR₈ₐC(═O)R_{8b}, —NR₈ₐS(═O)₂R_{8b}, —S(═O)₂R₈ₐ, —S(═O)₂NR₈ₐR_{8b}, —CN, —NO₂, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R₈ₐ and R_{8b} are independently selected from the group consisting of hydrogen, deuterium, and the following groups that are optionally substituted with one or more substituents: C₁₋₄ alkyl, C₃₋₆ cycloalkyl and C₃₋₆ cycloalkylmethylene, and the substituents are selected from the group consisting of: deuterium, halogen, —NH₂, —OH, —CN, C₁₋₄ alkyl, C₁₋₄ alkoxy, C₃₋₆ cycloalkyl and C₃₋₆ cycloalkylmethylene; the above substituents are optionally further substituted with one or more deuterium atoms or halogens.

52. The compound or the pharmaceutically acceptable salt thereof according to claim 51, wherein R₈ is selected from the group consisting of: 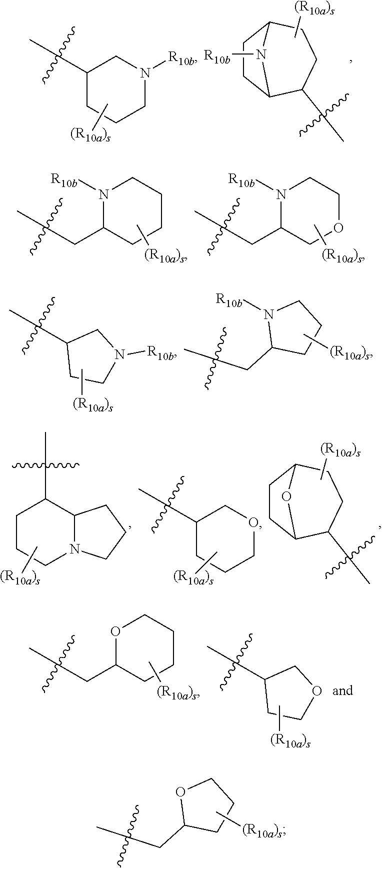
R₁₀ₐ is selected from the group consisting of hydrogen, deuterium, halogen, oxo, —OH, —NH₂, —COOH, —CN, C₁₋₄ alkyl and C₃₋₆ cycloalkyl, and the C₁₋₄ alkyl or C₃₋₆ cycloalkyl is optionally further substituted with one or more deuterium atoms, halogens or —OH;
R_{10b} is selected from the group consisting of hydrogen, deuterium, C₁₋₄ alkyl and C₃₋₆ cycloalkyl, and the C₁₋₄ alkyl and C₃₋₆ cycloalkyl are optionally further substituted with one or more deuterium atoms or halogens; s is an integer selected from 0-15.

53. (canceled)

54. (canceled)

55. The compound or the pharmaceutically acceptable salt thereof according to claim 1, wherein R₈ is selected from C₃₋₈ cycloalkyl that is optionally substituted with one or more substituents selected from the group consisting of deuterium, halogen, —OH, —NH₂, —CN and C₁₋₄ alkyl, and the C₁₋₄ alkyl is optionally further substituted with one or more of deuterium atoms, halogens or —OH.

56. The compound or the pharmaceutically acceptable salt thereof according to claim 55, wherein R₈ is selected from the group consisting of: 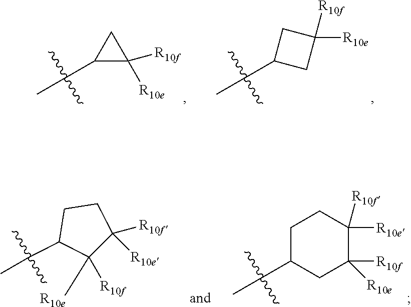
R₁₀ₑ, R_{10f}, R_{10e′} and R_{10f′} are independently selected from the group consisting of hydrogen, deuterium, halogen, —OH and C₁₋₄ alkyl, and the C₁₋₄ alkyl is optionally further substituted with one or more deuterium atoms, halogens or —OH.

57. (canceled)

58. (canceled)

59. A compound of formula I or a pharmaceutically acceptable salt thereof selected from the group consisting of:

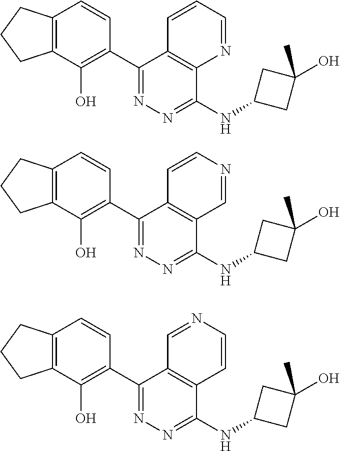

60. An isotopically substituted form of the compound according to claim 1, wherein the isotopic substitution is a substitution with a deuterium atom.

61. A pharmaceutical composition comprising the compound or the pharmaceutically acceptable salt thereof according to claim 1, and at least one pharmaceutically acceptable carrier, diluent or excipient.

62. A method for preventing and/or treating a disease associated with NLRP3 activity in a patient which comprises administering to the patient a therapeutically effective amount Hof the compound or the pharmaceutically acceptable salt thereof according to claim 1.

63. A method for preventing and/or treating inflammasome-related diseases, immune diseases, inflammatory diseases, autoimmune diseases and/or autoinflammatory diseases in a patient which comprises administering to the patient a therapeutically effective amount of the compound or the pharmaceutically acceptable salt thereof according to claim 1.

64. (canceled)

65. The compound or the pharmaceutically acceptable salt thereof according to claim 14, wherein R₂ and R₃, together with the atoms to which they are attached, form a 5-6 membered cyclic hydrocarbon; the 5-6 membered cyclic hydrocarbon is cyclopentyl or cyclohexyl; the cyclopentyl or cyclohexyl is optionally substituted with a substituent selected from the group consisting of hydrogen, deuterium, halogen, —OH, C₁₋₆ alkyl and C₁₋₆ haloalkyl.

66. The compound or the pharmaceutically acceptable salt thereof according to claim 14, wherein R₆ and R₇, together with the atoms to which they are attached, form 5-6 membered heteroaryl; the 5-6 membered heteroaryl is pyridine.

67. The compound or the pharmaceutically acceptable salt thereof according to claim 14, wherein R₄ and R₅ are independently selected from the group consisting of hydrogen and deuterium.

68. The compound or the pharmaceutically acceptable salt thereof according to claim 50, wherein n is 0.
